(19) United States
(12) Patent Application Publication
(10) Pub. No.: US 2021/0162030 A1
(43) Pub. Date: Jun. 3, 2021

(54) COMPOSITIONS AND METHODS FOR TREATMENT OF CANCER WITH LEKTI

(71) Applicant: Azitra Inc, Branford, CT (US)

(72) Inventor: Mark N. Sampson, Doylestown, PA (US)

(73) Assignee: Azitra Inc, Branford, CT (US)

(21) Appl. No.: 17/089,367

(22) Filed: Nov. 4, 2020

Related U.S. Application Data
(60) Provisional application No. 62/930,309, filed on Nov. 4, 2019.

(51) Int. Cl. A61K 38/55; A61P 35/00; C12N 15/86; A61K 35/742; A61K 35/745

(52) U.S. Cl. CPC A61K 38/55; A61P 35/00; A61K 35/745; A61K 35/742; C12N 15/86

(57) ABSTRACT

The present disclosure provides, inter alia, treating and/or preventing cancer and symptoms thereof, using recombinant LEKTI domains and microbes genetically modified to express one or more LEKTI protein domains encoded by one or more SPINK genes. In certain embodiments, compositions, methods, and kits are provided comprising recombinant LEKTI domains and microbes genetically modified to express one or more LEKTI protein domains encoded by one or more SPINK genes.

### RELATED APPLICATIONS

This application claims benefit under 35 U.S.C. § 119(e) of U.S. Provisional Application No. 62/930,309 filed Nov. 4, 2019, the contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 4, 2020, is named 129062-01102_SL.txt and is 59,655 bytes in size.

### FIELD OF THE DISCLOSURE

The present disclosure relates to methods, kits, and compositions for preventing or treating cancer in a subject using one or more therapeutic LEKTI domains.

### BACKGROUND OF THE INVENTION

Proteases or proteolytic enzymes are essential in organisms, from bacteria and viruses to mammals. Proteases digest and degrade proteins by hydrolyzing peptide bonds. Serine proteases (EC. 3.4.21) have common features in the active site, primarily an active serine residue. There are two main types of serine proteases; the chymotrypsin/trypsin/elastase-like or the subtilisin-like, which have an identical spatial arrangement of catalytic His, Asp, and Ser but in quite different protein scaffolds. Over twenty families (S1-S27) of serine proteases have been identified that are grouped into 6 clans on the basis of structural similarity and other functional evidence, SA, SB, SC, SE, SF & SG. The family of chymotrypsin/trypsin/elastase-like serine proteases have been subdivided into two classes. The “large” class (ca 230 residues) includes mostly mammalian enzymes such as trypsin, chymotrypsin, elastase, kallikrein, and thrombin. The “small” class (ca 190 residues) includes the bacterial enzymes. Examples of serine proteases include trypsin, tryptase, chymotrypsin, elastase, thrombin, plasmin, kallikrein, Complement Cl, acrosomal protease, lysosomal protease, cocoonase, a-lytic protease, protease A, protease B, serine carboxypeptidase t, subtilisin, urokinase (uPA), Factor Vila, Factor IXa, and Factor Xa. The serine proteases have been investigated extensively widely and are a major focus of research as a drug target due to their role in regulating a wide variety of physiological processes.

Serine protease inhibitors, or serpins, comprise a family of proteins that antagonize the activity of serine proteases. These proteins inhibit protease activity by a conserved mechanism involving a profound conformational change (as reviewed in Miranda and Lomas, 2006; Wang et al., 2008; and Ricagno et al., 2009). In this mechanism, the serpin presents a substrate-mimicking peptide sequence—the reactive center loop—to its target serine protease. Cleavage of the reactive center loop triggers a conformational change in which the bound protease translocates from the top to the bottom of the serpin molecule; simultaneously, part of the cleaved reactive center loop inserts into the β-sheet A of the serpin, thereby irreversibly inactivating the protease (Huntington et al., 2000; Briand et al., 2001).

One branch of the family of serine protease inhibitors is that of the Kazal type (SPINK) gene that includes SPINK1, SPINK2, SPINK4, SPINK5, SPINK6, SPINK7, SPINK8, SPINK9, SPINK13 and SPINK14. The SPINK family is the largest branch of the serine protease inhibitor family (Biagini et al. (2014) J Allergy Clin Immunol. 134: 891-899). The lymphoepithelial kazal-type inhibitor (LEKTI) is a multi-domain serine protease inhibitor encoded by SPINK5 (Serine Proteinase Inhibitor Kazal type 5) (Magert et al. (1999) J Biol. Chem. 274; 21499-21502). The SPINK5 gene encoding LEKTI is located on chromosome 5 among a cluster of other SPINK genes (e.g., SPINK1, SPINK6, SPINK7, SPINK9 and SPINK13), and comprises 33 exons encoding 15 inhibitory domains separated by linker regions. SPINK5 has been shown to be expressed in the skin, oral mucosa, tonsils, parathyroid gland, thymus, and lung (Magert et al., Int J Biochem Cell Biol. 2002; 34(6):573-6; Magert et al., Eur J Med Res. 2002; 7(2):49-56).

SPINK5 stands out among the other SPINK genes for the large number of inhibitory domains it encodes. Additionally, the SPINK5 gene is transcribed into three different transcripts, resulting in three different LEKTI proteins that differ in the C-terminal region; i.e., a 145 kDa full length protein having inhibitory domains D1-D15, a 125 kDa (short) protein having inhibitory domains D1-D12, and a 148 kDa (long) protein having an extended linker region 13. LEKTI is expressed as high molecular mass precursors, which are rapidly processed into several proteolytic fragments secreted in the intercellular space (Bitoun et al. (2003) Hum. Mol. Genet. 12:2417-2430). The Kazal motif of LEKTI is defined by the presence of six cysteine residues positioned at specific distances to allow formation of three disulfide bonds in a 1-5, 2-4, and 3-6 pattern. Two of the domains of LEKTI (D2 and D5) form this six cysteine motif, while other domains share four cysteine residues, which produce a rigid inhibitory loop believed to mimic the substrate of target proteases and inactivate the target protease catalytic site. The LEKTI protein requires proteolytic cleavage for activation of its inhibitory function against many proteases. The full length protein is cleaved into domains D1-D5 and D6-D15. The D6-D15 domains are then further cleaved in multiple steps into D6-D9 and D10-D15, 4 D6 and D7-D9→D7 and D8-D9→D8. This process results in LEKTI proteins comprising between one and six inhibitory domains, with each protein having different inhibitory functions. For example, it has been shown that LEKTI fragments can efficiently and specifically inhibit the epidermal kallikrein (KLK) 5, KLK7, and KLK14 (DeRaison et al. (2007) Mol. Biol. Cell. 18:3607-3619).

Jayakumar et al. ((2014) MOJ Proteomics Bioinform; 1(5):124-128) reported that recombinant human LEKTI inhibits a battery of serine proteinases in vitro including plasmin, trypsin, cathepsin G, human KLKs, and elastase, enzymes implicated in the activation of MMPs. The full-length LEKTI recombinant protein has been shown to inhibit trypsin, subtilisin A, plasmin, cathepsin G, and neutrophil elastase, but not chymotrypsin (Mitsudo et al. (2003) Biochemistry 42, 3874-3881). A partial recombinant form of LEKTI containing domains 6-9 (rLEKTI6-9) has been shown to inhibit trypsin, subtilisin A, chymotrypsin, kallikrein 5 (KLK5), and kallikrein 7 (KLK7), but not plasmin, cathepsin G, or elastase (Jayakumar et al. (2004) Protein Expr. Purif 35, 93-101; Schechter et al. (2005) Biol. Chem. 386, 1173-1184). In addition, the single domain D6 was shown to be a potent inhibitor of trypsin, KLK5, and KLK7, whereas D15 was not effective against these two kallikreins (Egelrud et al. (2005) Br. J. Dermatol. 153, 1200-1203). Deraison et al. (2007) Mol Biol Cell Vol. 18, 3607-3619) have identified KLK5 as a major target of LEKTI. While Deraison et al. demonstrated that all LEKTI fragments, except D1, demonstrate specific and differential inhibition of human kallikreins 5, 7, and 14, Jayakumar et al. (2014) found that the strongest inhibition was observed with D8-D11, toward KLK5, where kinetics analysis revealed an extremely tight binding complex. Thus, it has been reported that each form of LEKTI exhibits particular inhibitory specificity.

The main difference between a benign and malignant tumors is the ability of the malignant form to invade normal tissue and spread or metastasize to distant sites throughout the body. It is the ability to form metastasis which makes cancer such a difficult disease to treat. Evidence suggesting that proteolytic enzymes are involved in cancer spread, proteases are involved in normal destructive events and tissue remodeling, correlations exist between different protease activities and metastatic potential in model tumor systems, inhibitors and antibodies against proteases inhibit metastasis in model systems and the finding of highest levels of protease activity at the invading front in tumors. The most likely mechanism by which proteases could mediate metastasis is by catalyzing degradation of the extracellular matrix and basement membranes.

Primary (local) tumor cell invasion and metastasis occur by the attachment of tumor cells to components of the extracellular matrix (ECM) and by degradation of ECM by proteinase enzymes elaborated into the tumor microenvironment. These processes are regulated by proteolytic enzymes such as serine proteases, cysteine proteases, and matrix metalloproteinases (MMPs), tightly balanced by their endogenous inhibitors in the tumor microenvironment. Thus, inhibition of such proteinases can disrupt critical steps of primary tumor cell invasion and metastasis.

Jayakumar et al. (2014) demonstrated that stable expression of LEKTI in OSC-19 tongue squamous cell carcinoma cells resulted in markedly decreased levels of expression of genes encoding MMP-9, MMP-14, KLK5, and ADAMS. They found that LEKTI overexpressing cells displayed striking morphological changes and were more adhesive and less invasive. The results reported by Jayakumar et al. demonstrate a negative regulatory role for LEKTI in modulating the production of key MMPs involved in ECM degradation, and suggest that loss of LEKTI in head and neck squamous cell carcinoma (HNSCC) tumor cells could have an important role in HNSCC progression. Jayakumar et al. (2014) showed further in a xenograft model of tongue cancer, that tumors derived from LEKTI-expressing clones of an invasive head and neck cell line demonstrated limited pathologic features of lymphovascular and perineural invasion, again suggesting an important negative regulatory role for LEKTI in modulating extracellular matrix degradation.

Kallikreins are a family of proteases consisting of 15 closely related, secreted serine proteases with either trypsin-like or chymotrypsin-like specificity, are expressed in a variety of tissues such as prostate, ovary, breast, testis, brain, and skin. KLKs belong to a subgroup of the chymotrypsin-like serine protease family S1A of clan PA(S). The 15 human KLK genes are located on chromosome 19q13.4 and constitute the largest contiguous serine protease cluster in the human genome. These genes, generally composed of five coding exons and in some cases, one or two 5′ non-coding exons, encode the kallikrein-related peptidases KLK1 to KLK15. All KLK genes encode single-chain pre-pro-proteins containing a chymotrypsin- or trypsin-like catalytic domain of 224-237 residues with an amino acid sequence identity of approximately 40% among KLK4 to KLK15. KLK1 and its close homologs KLK2 and KLK3 form a clade of their own, KLK4, 5, and 7 belong to another subgroup, whereas KLK6 shares more similarity with KLK13 and KLK14. See Debela et al. (2008) Biol Chem 389, 623-632. KLKs are colocalized with LEKTI in skin (Ekholm et al., J Invest Dermatol, 114 (2000), pp. 56-63; Bitoun et al. Hum Mol Genet, 12 (2003), pp. 2417-2430, 2003; Komatsu et al. Br J Dermatol, 153 (2005), pp. 274-281). In addition, KLKs and LEKTI are secreted together in lamellar bodies to the intercellular space, in the uppermost Stratum granulosum (Sondell et al. J Invest Dermatol, 104 (1995), pp. 819-823; Ishida-Yamamoto et al. J Invest Dermatol, 122 (2004), pp. 1137-1144). KLKs are capable of cleaving corneodesmosomes, and their enzymatic activities are suppressed by partial recombinant LEKTI domains (Simon et al. J Biol Chem, 276 (2001), pp. 20292-20299; Caubet et al. J Invest Dermatol, 122 (2004), pp. 1235-1244; Egelrud et al. Br J Dermatol, 153 (2005), pp. 1200-1203; Schechter et al. Biol Chem, 386 (2005), pp. 1173-1184; Borgono et al. J Biol Chem, 282 (2007), pp. 3640-3652).

Human tissue KLKs have been shown to be aberrantly expressed in multiple malignancies including those of the prostate, breast, and ovary (Borgono et al. (2004) Mol Cancer Res. 2, 257-28010; Lawrence et al. (2010) Endocr. Rev. 31, 407-44611; Paliouras et al. (2007) Cancer Letts. 249, 61-79). A number of studies regarding KLK substrate specificity have revealed their association with the establishment and progression of malignancy. KLKs are implicated in proteolytic cascade pathways resulting to extended cleavage of extracellular matrix (ECM) components. It is well known that metastasis is associated with the invasive behaviors of tumor cells in which cell membrane proteins, receptors and ECM proteins play important roles. ECM degradation and remodeling, mediated directly by KLKs or via KLK-induced activation of other extracellular proteases, interrupt ECM physical barriers and cells' interaction, facilitating angiogenesis and cancer cells' invasiveness and metastasis (Borgoño and Diamandis (2004) Nat Rev Cancer. November; 4(11):876-90). Moreover, during the early stages of the disease, KLKs influence the availability of growth factors and therefore regulate tumor cell proliferation. For example, KLKs have been shown to be able to stimulate protease-activated receptors (PARs) through the cleavage of the extracellular N-terminal segment. PARs activation results in the triggering of an intracellular biochemical cascade leading to mitogen-activated protein kinase (MAPK) activation and cell proliferation (Sotiropoulou et al. (2009) J Biol Chem. 284(48):32989-94; Oikonomopoulou et al. (2010) Biol Chem. 391(4):299-310).

Kallikrein-related peptidase 5 (KLK5) is a secreted trypsin-like serine protease, encoded by the KLK5 gene of the KLKs gene family, under the transcriptional control of estrogens and progestins (Yousef G M, Diamandis E P (1999) J. Biol Chem. 274(53):37511-6). The proteolytic activity of KLK5 has been shown on a number of ECM components, such as collagens, fibronectin and laminin (Michael et al. (2005) J Biol Chem. 280(15):14628-35). KLKs are secreted as inactive zymogens (pro-KLKs) and their activation depends on the proteolytic cleavage of their N-terminus pro-peptide via autocatalysis or through other KLKs or non-KLK proteases (Borgoño et al. (2004) Mol Cancer Res. (5):257-80). The KLK5 auto activation from the secreted pro-KLK5 represents an initial process of the KLKs proteolytic cascade, triggering the activation of several other KLKs (KLK2, -3, -6, -7, -11, -12 and -14) (Michael et al. (2006) J Biol Chem. 281(18):12743-50). Thus, KLK5 has been considered to be the regulator of KLKs extracellular proteolytic cascade, counterbalancing the molecular microenvironment between normal physiology and cancer.

Jiang et al. ((2011) J. Biol. Chem. (11) 9127-9135) have demonstrated the effect of modulating KLK5 levels on the expression and integrity of desmoglein 1 (Dsg1) in normal oral keratinocytes and oral squamous cell carcinoma cells (OSCC). Their data demonstrated that malignant OSCC cells exhibit cleavage of Dsg1 which was blocked by treatment with proteinase inhibitors as well as by silencing of KLK5, and that modification of KLK5 expression also alters cell-cell aggregation and cohesion. These results suggest KLKs may contribute to metastatic dissemination of OSCC via a mechanism involving KLK5-catalyzed Dsg1 cleavage. Different groups have reported that the majority of the KLKs (KLK4-11, KLK13-15) are aberrantly expressed in ovarian cancers, compared to normal and benign tissues (Yousef et al. (2003) Cancer Res 63:2223-2227; Shih et al. (2004) Am J Pathol 164:1511-1518). High mRNA and/or protein levels of KLK4-7, KLK10 and KLK15 are associated with shorter progression-free and overall survival time of patients, and the up-regulated expression of KLK4-7, KLK10 and KLK15 is associated with high grade and late-stage disease, belonging to the more aggressive Type-II tumors (Bandiera et al. (2005) Int J Gynecol Cancer 19:1015-1021; Borgono et al. (2003) Int J Cancer 106:605-610; Scorilas et al. (2004) J Clin Oncol 22:678-685). Dong et al. (Clin Exp Metastasis (2014) 31:135-147) provide a review of the role of KLKs in epithelial ovarian cancer (EOC).

Among the pathways activated by KLKS are Par2, TSLP, Cathelicidin, and MMPs.

Proteinase-activated receptor 2 (PAR2) is a member of the G-protein coupled receptor 1 family (Nystedt et al. (1994) Proc Natl Acad Sci USA. 91(20):9208-12). PAR2 downstream signaling is mediated through several signaling pathways such as intracellular calcium, phospholipase C (PLC), mitogen-activated protein kinase (MAPK), Rho, and I-kappaB kinase/NF-kappaB (Ossovskaya V S, Bunnett N W (2004) Physiol Rev. 84(2):579-621; Grab et al. PLoS Negl Trop Dis. 3(7):e479). It is also transactivated by cleaved F2R/PAR1 (Lin H, Trejo J (2013) J Biol Chem. April 19); 288(16):11203-15).

PAR2 is known to regulate physiological responses such as vasoregulation, cell growth, inflammation, and nociception (Weithauser A, Rauch U Trends Cardiovasc Med. 2014 August; 24(6):249-55; Weithauser et al. J Am Coll Cardiol. 2013 Nov. 5; 62(19):1737-45). The PAR2 receptor has been shown to be involved in dermatitis, cell proliferation, cancer suppression, skin pigmentation, and skin moisture, and has been studied in the dermatology and cosmetic fields. PAR2 is activated by trypsin cleavage and coexists with tissue kallikrein in skin tissue. There is evidence that PAR2 also has an important role in tumors, especially in tumors of epithelial origin (Kanemaru et al. (2017) Int J Cancer. 140(1):130-141; Schaffner et al. (2010) December 23; 116(26):6106-13). Sun et al. (World J Gastroenterol. 2018 Mar. 14; 24(10): 1120-1133) found that PAR2 was upregulated in HCC tumor tissues and related with poor prognosis in HCC patients. Further, Sun et al. demonstrated that PAR2 could not only promote the proliferation and metastasis ability of SMMC-7721 and HepG2 cells in vitro, but also promoted xenograft tumor growth and HCC cell liver metastasis in vivo.

Cathelicidin proteins are composed of two distinct domains: an N-terminal “cathelin-like” or “prosequence” domain and the C-terminal domain of the mature anti-microbial peptide (AMP). The C-terminal domain of cathelicidins was among the earliest mammalian AMPs to show potent, rapid, and broad-spectrum killing activity. The term “cathelin-like” derives from the similarity of the N-terminal sequence with that of cathelin, a 12 kDa protein isolated from porcine neutrophils that share similarity with the cystatin superfamily of cysteine protease inhibitors.

The C-terminal 37 amino acids of human cathelicidin (LL-37) have been characterized. LL-37 was originally referred to as FALL39, named for the first 4 N-terminal amino acids of this domain and the total number of residues (i.e., 39). LL-37 is a peptide predicted to contain an amphipathic alpha-helix and lacks cysteine, making it different from all other previously isolated human peptide antibiotics of the defensin family, each of which contain 3 disulfide bridges. Full length human cathelicidin (sometimes referred to as full length LL-37) comprises the cathelin-like precursor protein and the C-terminal LL-37 peptide, thus comprising 170 amino acids.

An increasing amount of evidence suggests that LL-37 can have two different and contradictory effects—promotion or inhibition of tumor growth. The mechanisms are tissue-specific, complex, and depend mostly on the ability of LL-37 to act as a ligand for different membrane receptors whose expression varies on different cancer cells. LL-37-induced apoptosis explains its antitumor activity in colon cancers and hematologic malignancies (Mader et al. 2009 Mol Cancer Res. 7(5):689-702; Ren et al. 2012 Cancer Res. 72(24):6512-23; Ren et al. 2013 PLoS One. 8(5):e636412013). However, LL-37 can also promote tumor growth, depending on the tissue from which the cancer cells originate. In various types of cancer a different expression of LL-37 peptide was observed. In ovarian, lung, breast cancer and malignant melanoma cells, an increase in LL-37 expression was reported (Bals et al. 1998 Proc Natl Acad Sci USA. August 4; 95(16):9541-6; Coffelt et al. 2008 Int J Cancer. March 1; 122(5):1030-9; Heilborn et al. 2005 Int J Cancer. May 1; 114(5):713-9; Kim et al. 2010 Br J Dermatol. 163(5):959-67). Increased expression of LL-37 has been observed in breast cancer cells, with secreted concentrations correlating to phenotypic tumor severity (Heilborn et al. 2005 Int J Cancer. 114(5):713-9.). Evaluation of hCAP18/LL-37 ability to promote breast cancer development revealed that metastatic potential greatly increases as a result of augmented Heregulin-mediated mitogenic signaling through ErbB2. A modified version of LL-37 competitively inhibited LL-37 induced MAPK phosphorylation and drastically reduced cancer cell colonies induced by LL-37, in addition to inhibiting cancer cell migration (Weber et al. 2009 Breast Cancer Res. 11(1):R6). In contrast, cells from colon or gastric cancers produce lower amounts of this peptide (Hase et al. 2003 Gastroenterology. 2003 December; 125(6):1613-25; Ren et al. 2012 Cancer Res. 2012 Dec. 15; 72(24):6512-23) Immunohistochemical analysis of LL-37 expression in skin tumors revealed that there might be a relationship between the level of hCAP-18/LL-37 and the development of cancerous conditions in these cells. Several in vitro studies indicate that secretion of LL-37 in malignant melanoma cells is significantly increased compared to normal skin and hematological malignant cell lines, suggesting the possibility that LL-37 acts as a growth factor for skin tumor cells and enhances cancer development (Chen et al. (2018) Cell Physiol Biochem. 47(3):1060-107). Taken together, these observations imply that the actions of LL-37 are tissue-specific.

There remains a need to develop improved agents for therapeutic, prophylactic or diagnostic approaches for the treatment of cancer.

### SUMMARY

The present disclosure is based, in part, on treating, preventing or delaying the progression of cancer, and in some embodiments, on treating, preventing or delaying the progression of tumor cell invasion and metastasis, by inhibiting pathways activated by protease targets of LEKTI. Without being bound by theory, the present disclosure is based on the finding that certain LEKTI fragments can selectively inhibit KLK5 activity, and, in turn, inhibit various proteases and proteolytic pathways implicated in cancer development and progression. Accordingly, LEKTI therapy can target pathways involved in epithelial to mesenchymal transition, thereby inhibiting the malignant phenotype. Moreover, LEKTI therapy, as described herein, advantageously targets pathways involved in apoptosis, cell cycle arrest and/or cell proliferation.

According to one aspect, the disclosure provides a method of treating a subject afflicted with a cancer, comprising administering a one or more LEKTI protein domains to the subject in need thereof.

According to one aspect, the disclosure provides a method of preventing the recurrence of a cancer in a subject afflicted with the cancer, wherein the cancer is in remission, comprising administering one or more LEKTI protein domains to the subject in need thereof.

According to one aspect, the disclosure provides a method of preventing the progression of a cancer in a subject afflicted with the cancer, comprising administering one or more LEKTI protein domains to the subject in need thereof.

According to one aspect, the disclosure provides a method of preventing a cancer in a subject with risk factors for developing the cancer, comprising administering one or more LEKTI protein domains to the subject in need thereof.

According to one aspect, the disclosure provides a method for inhibiting serine protease activity of at least one serine protease in a subject afflicted with cancer, comprising administering one or more LEKTI protein domains to the subject in need thereof.

According to one aspect, the disclosure provides a method of treating a subject afflicted with a cancer, comprising administering a microbe comprising one or more LEKTI protein domains to the subject in need thereof.

According to some embodiments, the one or more LEKTI protein domains are encoded by a nucleic acid. According to some embodiments, the nucleic acid comprises SEQ ID NO: 119, or fragments thereof. According to some embodiments, the nucleic acid comprises a sequence that is at least 80%, 85%, 90%, 95%, 96%, 97%, 98% or 99% identical to SEQ ID NO: 119. According to some embodiments, the nucleic acid comprises a sequence that is at least 80%, 85%, 90%, 95%, 96%, 97%, 98% or 99% identical to SEQ ID NO: 128. According to some embodiments, the nucleic acid consists of SEQ ID NO: 119. According to some embodiments, the nucleic acid consists of SEQ ID NO: 128. According to another aspect, the nucleic acid is comprised in a vector. According to some embodiments, the vector is a viral expression vector. According to some embodiments, the vector is comprised within a cell.

According to one aspect, the disclosure provides a method of preventing the recurrence of a cancer in a subject afflicted with the cancer, wherein the cancer is in remission, comprising administering a microbe comprising one or more LEKTI protein domains to the subject in need thereof.

According to one aspect, the disclosure provides a method of preventing the progression of a cancer in a subject afflicted with the cancer, comprising administering a microbe genetically modified to express one or more LEKTI protein domains encoded by one or more SPINK genes to the subject in need thereof.

According to one aspect, the disclosure provides a method of preventing a cancer in a subject with risk factors for developing the cancer, comprising administering a microbe genetically modified to express one or more LEKTI protein domains encoded by one or more SPINK genes to the subject in need thereof.

According to one aspect, the disclosure provides a method for inhibiting serine protease activity of at least one serine protease in a subject afflicted with cancer, comprising administering a microbe genetically modified to express one or more LEKTI protein domains encoded by one or more SPINK genes to the subject in need thereof.

According to some embodiments of any of the above aspects, the cancer is selected from the group consisting of: malignant melanoma, colon cancer, breast cancer, lung cancer, ovarian cancer, gastric cancer, oral tongue squamous cell carcinoma, squamous cell cancer, prostate cancer, pancreatic cancer, liver cancer, kidney cancer, bladder cancer, cervical cancer, endometrial cancer, gallbladder cancer, brain cancer and oral cancer. According to some embodiments of any of the above aspects, the cancer is malignant. According to some embodiments of any of the above aspects, the cancer is in a precancerous stage. According to some embodiments of any of the above aspects, the method increases the duration of survival of the subject. According to some embodiments of any of the above aspects, the method increases the progression-free survival of the subject. According to some embodiments of any of the above aspects, the method increases the progression-free survival of the subject in comparison to standard-of-care therapies. According to some embodiments of any of the above aspects, the method is part of a therapeutic regimen combining one or more additional treatment modalities.

According to some embodiments of any of the above aspects, the microbe is adapted to live for a controlled duration on the surface of the mammal's skin to provide a continuous supply of LEKTI protein domains. According to some embodiments of any of the above aspects, the microbe is genetically modified by transfection/transformation with a recombinant DNA plasmid encoding the LEKTI protein domains. According to some embodiments of any of the above aspects, the LEKTI domains are operably linked to one or more recombinant protein domains that are effective to enhance secretion from the microbe and/or penetration of the mammal's skin. According to some embodiments of any of the above aspects, at least one LEKTI domain is operably linked to a SecA domain.

According to some embodiments of any of the above aspects, the at least one LEKTI domain is operably linked to an RMR domain. According to some embodiments of any of the above aspects, the at least one LEKTI domain comprises an amino acid sequence comprising any one of SEQ ID NOs 104-118 (any one of SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118). According to some embodiments, the at least one LEKTI domain comprises an amino acid sequence that is at least 85% identical, at least 90% identical, at least 95% identical, at least 96% identical, at least 97% identical, at least 98% identical or at least 99% identical to SEQ ID NOs 104-118 (at least 85% identical, at least 90% identical, at least 95% identical, at least 96% identical, at least 97% identical, at least 98% identical or at least 99% identical to SEQ ID NO: 105, at least 85% identical, at least 90% identical, at least 95% identical, at least 96% identical, at least 97% identical, at least 98% identical or at least 99% identical to SEQ ID NO: 106, at least 85% identical, at least 90% identical, at least 95% identical, at least 96% identical, at least 97% identical, at least 98% identical or at least 99% identical to SEQ ID NO: 107, at least 85% identical, at least 90% identical, at least 95% identical, at least 96% identical, at least 97% identical, at least 98% identical or at least 99% identical to SEQ ID NO: 108, at least 85% identical, at least 90% identical, at least 95% identical, at least 96% identical, at least 97% identical, at least 98% identical or at least 99% identical to SEQ ID NO: 109, at least 85% identical, at least 90% identical, at least 95% identical, at least 96% identical, at least 97% identical, at least 98% identical or at least 99% identical to SEQ ID NO: 110, at least 85% identical, at least 90% identical, at least 95% identical, at least 96% identical, at least 97% identical, at least 98% identical or at least 99% identical to SEQ ID NO: 111, at least 85% identical, at least 90% identical, at least 95% identical, at least 96% identical, at least 97% identical, at least 98% identical or at least 99% identical to SEQ ID NO: 112, at least 85% identical, at least 90% identical, at least 95% identical, at least 96% identical, at least 97% identical, at least 98% identical or at least 99% identical to SEQ ID NO: 113, at least 85% identical, at least 90% identical, at least 95% identical, at least 96% identical, at least 97% identical, at least 98% identical or at least 99% identical to SEQ ID NO: 114, at least 85% identical, at least 90% identical, at least 95% identical, at least 96% identical, at least 97% identical, at least 98% identical or at least 99% identical to SEQ ID NO: 115, at least 85% identical, at least 90% identical, at least 95% identical, at least 96% identical, at least 97% identical, at least 98% identical or at least 99% identical to SEQ ID NO: 116, at least 85% identical, at least 90% identical, at least 95% identical, at least 96% identical, at least 97% identical, at least 98% identical or at least 99% identical to SEQ ID NO: 117 or at least 85% identical, at least 90% identical, at least 95% identical, at least 96% identical, at least 97% identical, at least 98% identical or at least 99% identical to SEQ ID NO: 118). According to some embodiments, the at least one LEKTI domain comprises SEQ ID NO: 109. According to some embodiments, the at least one LEKTI domain consists of SEQ ID NO: 109. According to some embodiments of any of the above aspects, the microbe is adapted to multiply on the skin of the mammal According to some embodiments of any of the above aspects, the expression of at least one LEKTI domain is controlled by an operon and the amount of LEKTI provided to the subject's skin is proportional to the availability of an extrinsic factor. According to some embodiments of any of the above aspects, the expression of at least one LEKTI domain is controlled by a promoter that is constitutively active. According to some embodiments of any of the above aspects, the microbe has been genetically modified by transfection/transformation with a recombinant DNA plasmid encoding the one or more LEKTI protein domains and one or more antibiotic resistance genes. According to some embodiments of any of the above aspects, the microbe is selected from the group consisting of Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp. According to some embodiments, bacteria in the microbial compositions comprise one or more of Staphylococcus epidermidis, Staphylococcus hominis, Staphylococcus warneri, Streptococcus pyogenes, Streptococcus mitis, Lactobacillus acidophilus, Propionibacterium acnes, Acinetobacter johnsonii, and Pseudomonas aeruginosa and mixtures thereof.

According to some embodiments of any one of the above aspects, the microbe is a Staphylococcus spp.

According to one aspect, the disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide. According to one embodiment, the pharmaceutical composition comprises the recombinant microorganism.

### BRIEF DESCRIPTION OF THE DRAWINGS

FIG. 1 shows a vector construct comprising the therapeutic LEKTI domains of the present invention. The protein coding regions of the plasmid comprise SecA, 6×His tag (SEQ ID NO: 120), LEKTI D8-11, and RMR tag, operably linked to each other and under the control of a CmR promoter.

FIG. 1 discloses SEQ ID NOS 122-125, respectively, in order of appearance.

FIG. 2 shows a vector construct of the pJB38 plasmid according to some embodiments of the present invention.

FIG. 3 is a schematic showing the domains of the full length LEKTI polypeptide.

FIG. 4 is a graph showing exogenous LEKTI D6 partially blocks EGF-induced invasion of OSC-19 cells.

FIG. 5 is a graph showing exogenous LEKTI D6 potently blocks EGF-induced invasion of OSC-19 cells.

FIG. 6 is a graph showing the dose dependent effect of exogenous LEKTI D6 on EGF-induced invasion of OSC-19 cells.

FIG. 7 is a schematic of the upper and lower chambers of a multiwall plate showing the effect of cell density and EGF on Normal Human Epidermal Keratinocyte (NHEK) migration.

FIG. 8 is a schematic of the upper and lower chambers of a multiwall plate showing the effect of escalating doses of LEKTI D6 on NHEK migration.

### DETAILED DESCRIPTION OF THE INVENTION

Aspects of the present disclosure provide LEKTI proteins, or portions thereof, that are administered to a subject for the treatment of cancer or a precancerous condition.

According to some embodiments, the disclosure provides bacteria that are genetically modified to express one or more LEKTI protein domains encoded by one or more SPINK genes. The protein-producing bacteria are able to treat, prevent or delay the progression of cancer in a subject by expressing and, optionally, secreting a therapeutic protein that inhibits pathways activated by protease targets of LEKTI. According to some embodiments, the protein-producing bacteria are able to treat, prevent or delay the progression of tumor cell invasion and/or tumor cell metastasis, by expressing and, optionally, secreting a therapeutic protein that inhibits pathways activated by protease targets of LEKTI. According to some embodiments, the therapeutic protein comprises one or more LEKTI domains that are effective to inhibit serine proteases within or on the skin of a mammal According to some embodiments, the recombinant LEKTI domains compensate for the defective endogenous LEKTI protein naturally produced by the skin in the mammal. According to some embodiments, the bacteria are able to self-replicate while retaining the ability to produce the recombinant protein, thereby providing a continuous supply of therapeutic agent.

According to some embodiments, the disclosure provides a composition to treat, prevent or delay the progression of cancer in a subject, comprising a microbe genetically modified to express one or more LEKTI protein domains encoded by one or more SPINK genes that is administered to the subject. According to some embodiments, the disclosure provides a composition to treat, prevent or delay the progression of tumor cell invasion and/or tumor cell metastasis in a subject, comprising a microbe genetically modified to express one or more LEKTI protein domains encoded by one or more SPINK genes that is administered to the subject.

According to some embodiments, the disclosure provides a composition to treat, prevent or delay the progression of cancer in a subject, comprising a microbe genetically modified to express one or more LEKTI protein domains encoded by one or more SPINK genes that is administered onto the skin of the subject, wherein the LEKTI protein domains are effective to penetrate one or more layers of the subject's skin and effective to inhibit serine protease activity of at least one serine protease in or on the subject's skin. According to some embodiments, the disclosure provides a composition to treat, prevent or delay the progression of tumor cell invasion and/or tumor cell metastasis in a subject, comprising a microbe comprising one or more LEKTI protein domains onto the skin of the subject, wherein the LEKTI protein domains are effective to penetrate one or more layers of the subject's skin and effective to inhibit serine protease activity of at least one serine protease in or on the subject's skin.

### 1. Definitions

As used herein and in the appended claims, the singular forms “a,” “and,” and “the” include plural referents unless the context clearly dictates otherwise. Thus, for example, reference to “a polynucleotide” includes a plurality of such polynucleotides and reference to “the polypeptide” includes reference to one or more polypeptides, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “administration” as used herein is meant to refer to contact of a pharmaceutical composition, therapeutic composition, diagnostic agent or composition to a recipient, preferably a human.

The term “biological sample” as used herein is meant to refer to a sample that may be extracted, untreated, treated, diluted or concentrated from a subject. Suitably, the biological sample is selected from tissue samples including tissue from the ovaries, endometrium, and prostate. The biological sample may also be a fluid selected from the group consisting of whole blood, serum, plasma, saliva, urine, sweat, ascitic fluid, peritoneal fluid, synovial fluid, amniotic fluid, cerebrospinal fluid, skin biopsy, and the like. The biological sample may include serum, whole blood, plasma, lymph and ovarian follicular fluid as well as other circulatory fluid and saliva, mucus secretion and respiratory fluid.

As used herein, the terms “disease” or “disorder” are meant to refer to an impairment of health or a condition of abnormal functioning.

As used herein, an “effective amount” or “a therapeutically effective amount” of an agent, e.g., a pharmaceutical formulation, is meant to refer to an amount effective, at dosages and for periods of time necessary, to achieve the desired therapeutic result. A “therapeutically effective amount” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. By way of example for the treatment of tumors, a therapeutically effective amount of an anti-cancer agent can inhibit cell growth or tumor growth by at least about 20%, by at least about 40%, by at least about 60%, or by at least about 80% relative to untreated subjects. In other embodiments of the invention, tumor regression can be observed and continue for a period of at least about 20 days, at least about 40 days, or at least about 60 days. A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In certain embodiments, the prophylactically effective amount prevents the development or recurrence of the cancer entirely.

As used herein, the terms “gene” or “coding sequence,” is meant to refer broadly to a DNA region (the transcribed region) which encodes a protein. A coding sequence is transcribed (DNA) and translated (RNA) into a polypeptide when placed under the control of an appropriate regulatory region, such as a promoter. A gene may comprise several operably linked fragments, such as a promoter, a 5′-leader sequence, a coding sequence and a 3′-non-translated sequence, comprising a polyadenylation site. The phrase “expression of a gene” refers to the process wherein a gene is transcribed into an RNA and/or translated into an active protein.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence.

As used herein, the term “functional variant of a gene” includes a variant of the gene with minor variations such as, for example, silent mutations, single nucleotide polymorphisms, missense mutations, and other mutations or deletions that do not significantly alter gene function.

As used herein, the term “gene delivery” means a process by which foreign DNA is transferred to host cells for applications of gene therapy.

As used herein, the term “gene of interest (GOI),” as used herein refers broadly to a heterologous sequence introduced into an expression vector, and typically refers to a nucleic acid sequence encoding a protein of therapeutic use in humans or animals.

The term “genetically modified” and grammatical variations thereof as used herein are meant to describe a microbial organism (e.g. bacteria) that has been genetically modified or engineered by the introduction of DNA prepared outside the microbe. For example, the introduction of plasmid DNA containing new genes into bacteria will allow the bacteria to express those genes. Alternatively, the DNA containing new genes can be introduced to the bacteria and then integrated into the bacteria's genome, where the bacteria will express those genes.

As used herein, the term “heterologous,” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide) Similarly, a cellular sequence (e.g., a gene or portion thereof) that is incorporated into a viral vector is a heterologous nucleotide sequence with respect to the vector.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, the term “infection,” is meant to refer broadly to delivery of heterologous DNA into a cell by a virus. The term “co-infection” as used herein means “simultaneous infection,” “double infection,” “multiple infection,” or “serial infection” with two or more viruses. Infection of a producer cell with two (or more) viruses will be referred to as “co-infection.” The term “transfection” refers to a process of delivering heterologous DNA to a cell by physical or chemical methods, such as plasmid DNA, which is transferred into the cell by means of electroporation, calcium phosphate precipitation, or other methods well known in the art.

The term “inhibiting” as used herein in reference to the development or recurrence of a cancer means either lessening the likelihood of the cancer's development or recurrence, or preventing the development or recurrence of the cancer entirely.

As used herein, the term “isolated” molecule (e.g., an isolated nucleic acid or protein or cell) means it has been identified and separated and/or recovered from a component of its natural environment.

The terms “metastasis” or “metastases” as used herein refer to tumor growth or deposit that has spread via lymph or blood to an area of the body remote from the primary tumor.

The “term melanoma” as used herein is used in the broadest sense and refers to all stages and all forms of cancer arising from melanocytes. Melanoma is typically a malignant tumor associated with skin cancer.

The term “metastasize” as used herein refers to the spread of cancer from one part of the body to another. A tumor formed by cells that have spread is called a “metastatic tumor” or “metastasis.” The plural form of “metastasis” is “metastases.”

As used herein, the term “minimal regulatory elements” is meant to refer to regulatory elements that are necessary for effective expression of a gene in a target cell and thus should be included in a transgene expression cassette. Such sequences could include, for example, promoter or enhancer sequences, a polylinker sequence facilitating the insertion of a DNA fragment within a plasmid vector, and sequences responsible for intron splicing and polyadenlyation of mRNA transcripts.

As used herein, a “nucleic acid” or a “nucleic acid molecule” is meant to refer to a molecule composed of chains of monomeric nucleotides, such as, for example, DNA molecules (e.g., cDNA or genomic DNA). A nucleic acid may encode, for example, a promoter, the LEKTI gene or portion thereof (e.g., LEKTI D6), or regulatory elements. A nucleic acid molecule can be single-stranded or double-stranded. A “LEKTI nucleic acid” refers to a nucleic acid that comprises the LEKTI gene or a portion thereof, or a functional variant of the LEKTI gene or a portion thereof. A functional variant of a gene includes a variant of the gene with minor variations such as, for example, silent mutations, single nucleotide polymorphisms, missense mutations, and other mutations or deletions that do not significantly alter gene function.

The asymmetric ends of DNA and RNA strands are called the 5′ (five prime) and 3′ (three prime) ends, with the 5′ end having a terminal phosphate group and the 3′ end a terminal hydroxyl group. The five prime (5′) end has the fifth carbon in the sugar-ring of the deoxyribose or ribose at its terminus. Nucleic acids are synthesized in vivo in the 5′- to 3′-direction, because the polymerase used to assemble new strands attaches each new nucleotide to the 3′-hydroxyl (—OH) group via a phosphodiester bond.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

A DNA sequence that “encodes” a particular LEKTI (e.g., LEKTI D6) protein is a nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

The term “operably linked” as used herein is meant to refer to the association of nucleic acid sequences on a single nucleic acid fragment so that the function of one is regulated by the other or is not hindered by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, two proteins can be operably linked, such that the function of either protein is not compromised. Generally, operably linked means that the nucleic acid sequences being linked are contiguous and, where necessary to join two protein coding regions, contiguous and in the same reading frame.

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. An example of an alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

The term “pharmaceutical formulation” as used herein is meant to refer to a preparation which is in such form as to permit the biological activity of an active ingredient contained therein to be effective, and which contains no additional components which are unacceptably toxic to a subject to which the formulation would be administered.

A “pharmaceutically acceptable carrier” as used herein is meant to refer to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a “promoter” is meant to refer to a region of DNA that facilitates the transcription of a particular gene. As part of the process of transcription, the enzyme that synthesizes RNA, known as RNA polymerase, attaches to the DNA near a gene. Promoters contain specific DNA sequences and response elements that provide an initial binding site for RNA polymerase and for transcription factors that recruit RNA polymerase. According to some embodiments, the promoter is selected from the group consisting of a CBA promoter, smCBA promoter, a CASI promoter, a GFAP promoter, and an elongation factor-1 alpha (EF1a) promoter. A “chicken beta-actin (CBA) promoter” refers to a polynucleotide sequence derived from a chicken beta-actin gene (e.g., Gallus beta actin, represented by GenBank Entrez Gene ID 396526). A “smCBA” promoter refers to the small version of the hybrid CMV-chicken beta-actin promoter. A “CASI” promoter refers to a promoter comprising a portion of the CMV enhancer, a portion of the chicken beta-actin promoter, and a portion of the UBC enhancer.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art.

The term “progression” as used herein refers to the course of a disease, such as cancer, as it becomes worse or spreads in the body.

The term “progression free survival” or “PFS” as used herein refers to a length of time during and after the treatment of a disease, such as cancer, that a patient lives with the disease but it does not get worse. In a clinical trial, measuring the progression free survival is one way to determine how well a new treatment works.

The terms “protease” and “proteinase” as used herein are interchangeable, with both terms referring to an enzyme that performs proteolysis.

The term “quality of life” as used herein refers to the overall enjoyment of life, including aspects of an individual's sense of well-being and ability to carry out various activities.

The terms “recurrence” or “relapse” are used interchangeably herein to refer to the return of a cancer after a first-line treatment and after a period of time during which the cancer cannot be detected.

The term “refractory” as used herein refers to cancer that does not respond to treatment. The cancer may be resistant at the beginning of treatment or it may become resistant during treatment. The term “primary refractory” as used herein refers to the progression of disease during induction treatment or a partial or transient response (e.g. less than 60 days) to induction therapy. The term “induction therapy” as used herein refers to the first treatment given for a disease which is often part of a standard set of treatments, for example, surgery followed by chemotherapy and radiation. Induction therapy is often accepted as the best treatment option. Induction therapy is also known as “first-line therapy,” “primary therapy” and “primary treatment.”

The term “relapse-free survival (RFS)” as used herein refers to the length of time after primary treatment for a cancer during which the patient survives without any signs or symptoms of that cancer. It is also called disease-free survival (DFS).

The term “recombinant” and grammatical variations thereof are meant to relate to or denote an organism, protein, or genetic material formed by or using recombined DNA comprising DNA pieces from different sources or from different parts of the same source. For example, the term “recombinant DNA” means a DNA molecule formed through recombination methods to splice fragments of DNA from a different source or from different parts of the same source. According to some embodiments, two or more different sources of DNA are cleaved using restriction enzymes and joined together using ligases. As another example, the term “recombinant protein” or “recombinant domains” and grammatical variations thereof means a protein molecule formed through recombination methods originating from spliced fragments of DNA from a different source or from different parts of the same source. As another example, the term “recombinant microbe” or “recombinant bacteria” and grammatical variations thereof mean a microbe/bacteria that comprises one or more recombinant DNA/protein molecules.

The term “risk factor” as used herein refers to anything that raises the chances of a person developing a disease or disorder.

As used herein the term “secretory peptides” or “secretory sequences” or “secretion tags” or “signal peptides” or “export signals” and grammatical variations thereof means any peptide sequence that is capable of targeting the synthesized protein to the secretory pathway of a cell.

The term “skin” as used herein is meant to refer to the outer protective covering of the body of a mammal (e.g., a human), consisting of the corium and the epidermis, and is understood to include sweat and sebaceous glands, as well as hair follicle structures. Throughout the disclosure, the adjective “cutaneous” can be used, and should be understood to refer generally to attributes of the skin, as appropriate to the context in which they are used.

The term “subject” as used herein is meant to refer to a mammal Mammals include, but are not limited to, domesticated animals (e.g., cows, sheep, cats, dogs, and horses), primates (e.g., humans and non-human primates such as monkeys), rabbits, and rodents (e.g., mice and rats). According to some embodiments, the subject is a human. The term “subject” is used interchangeably with the term “patient” herein. According to some embodiments, the subject is suspected of having or being pre-disposed to a cancer as described herein.

As used herein, the term “transgene” is meant to refer to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

As used herein, a “transgene expression cassette” or “expression cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The term “treatment” (and variations such as “treat” or “treating”) as used herein is meant to refer clinical intervention in an attempt to alter the natural course of the subject or cell being treated. Desirable effects of treatment include one or more of preventing occurrence or recurrence of disease, alleviation of symptoms, diminishment of any direct or indirect pathological consequences of the disease, stabilized (i.e., not worsening) state of disease, decreasing the rate of disease progression, amelioration or palliation of the disease state, prolonging survival as compared to expected survival if not receiving treatment and improved prognosis.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “recombinant viral vector” is meant to refer to a recombinant polynucleotide vector comprising one or more heterologous sequences (i.e., nucleic acid sequence not of viral origin).

As used herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

Transcriptional regulators refer to transcriptional activators and repressors that either activate or repress transcription of a gene of interest, such as LEKTI (e.g., LEKTI D6). Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### 2. Compositions

According to some aspects, the present disclosure provides compositions comprising a microbe genetically modified to express one or more LEKTI protein domains encoded by one or more SPINK genes. According to some embodiments, the one or more SPINK genes are selected from the group consisting of SPINK1, SPINK2, SPINK4, SPINK5, SPINK6, SPINK7, SPINK8, SPINK9, SPINK13, and SPINK14. According to some embodiments, the SPINK gene is SPINK5.

The SPINK gene can be obtained from any mammal, such as mouse, rat, rabbit, goat, sheep, horse, cow, dog, primate, or human gene sequences. According to some embodiments, the SPINK gene sequence is a human gene sequence.

According to some embodiments, the present disclosure also provides recombinant vectors containing a nucleotide sequence encoding one or more SPINK genes or portions thereof. Recombinant vectors include but are not limited to vectors useful for the expression of the open reading frames (ORFs) in E. coli, other bacteria, yeast, viral, baculovirus, plants or plant cells, as well as mammalian cells.

As described herein, according to some embodiments, the disclosure provides a microbe that is genetically modified to express one or more protein domains encoded by one or more SPINK genes to the subject in need thereof. According to some embodiments, the recombinant microbe is engineered to comprise a SPINK gene, or a fragment of the SPINK gene.

According to some embodiments, the virus is an oncolytic virus. Oncolytic viruses (OVs) can replicate in cancer cells but not in normal cells, leading to lysis of the tumor mass. Beside this primary effect, OVs can also stimulate the immune system. Tumors are an immuno-suppressive environment in which the immune system is silenced in order to avoid the immune response against cancer cells. The delivery of OVs into the tumor results in a strong and durable immune response against the tumor itself. Suitable expression vectors for expression in a suitable host are known to one skilled in the art and appropriate expression vectors can be obtained from commercial sources or from the American Type Culture Collection (ATCC).

Useful embodiments include, for example, promoter sequences operably linked to the ORF, regulatory sequences, and transcription termination signals.

According to some embodiments, the nucleic acids have been appropriately modified, for example, by site directed mutagenesis, to remove sequences responsible for N-glycosylation not needed for biological activity. N-glycosylation sites in eukaryotic peptides are characterized by the amino acid sequence Asn-X-Ser/Thr where X is any amino acid except Pro. Modification of glycosylation sites can improve expression in for example yeast or mammalian cell cultures.

According to some embodiments, the nucleic acids have been modified to improve the production and solubility of recombinant protein in a suitable host which includes, but is not limited to removing cysteine residues unnecessary for intramolecular disulfide bond formation. cysteine residues may be changed by mutagenesis to another amino acid, for example serine, or removed from the sequence without affecting the biological activity or tertiary structure of the recombinant polypeptide.

Other modifications of the nucleic acids may be necessary to improve the stability and accumulation of the recombinant production of protein include but are not limited to mutations altering protease cleavage sites recognized by a suitable expression host. Such modifications can be made that will not adversely affect the biological activity or tertiary structure of the recombinant protein.

Additional modifications can be made to the nucleic acids that result in alterations in enzyme activity, substrate specificity, and/or biological activity. Such modifications may be preconceived based on specific knowledge relating to the protein or may be introduced by a random mutagenesis approach, for example error prone PCR. Additionally, it is also envisioned that one skilled in the art could generate chimeric nucleotide sequence comprising specific domains that can functionally replace stretches of nucleotide sequences that may add new function or improve the specificity or activity of the produced recombinant protein. According to some embodiments, modification resulting in changed biological activity of LEKTI may be necessary to improve the therapeutic effectiveness of the protein or to minimize potential side effects. Modification of the nucleic acid sequences can also be made that alter potential immunogenic sites that may result in allergic reactions to patients' administered with recombinant LEKTI protein.

Silent modifications can be made to the nucleic acids that do not alter, substitute or delete the respective amino acid in the recombinant protein. Such modification may be necessary to optimize, for example, the codon usage for a specific recombinant host. The nucleotide sequence of LEKTI or portions thereof can be modified to replace codons that are considered rare or have a low frequency of appropriate t-RNA molecules to a more suitable codon appropriate for the expression host. Such codon tables are known to exist and are readily available to one skilled in the art. In addition, silent modification can be made to the nucleic acid that minimizes secondary structure loops at the level of mRNA that may be deleterious to recombinant protein expression.

According to some embodiments, the one or more SPINK genes encodes a LEKTI protein, and protein domains thereof, selected from LEKTI, LEKTI-2 and LEKTI-3.

The present disclosure provides compositions comprising a therapeutically effective amount of a LEKTI polypeptide or a portion thereof. According to some embodiments, a portion of a LEKTI polypeptide comprises one or more LEKTI protein domains. Accordingly, according to some embodiments, the present disclosure provides compositions comprising one or more LEKTI protein domains. Some non-limiting examples include one or more of domains D1, D2, D3, D4, D5, D6, D7, D8, D9, D10, D11, D12, D13, D14, and D15. According to some embodiments, the LEKTI domain comprises LEKTI inhibitory domain 6 (LEKTI D6).

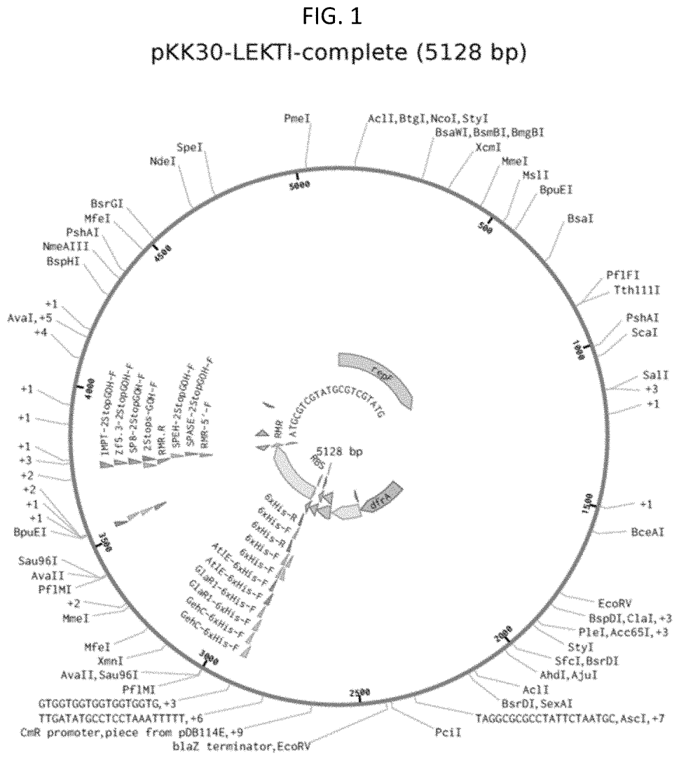

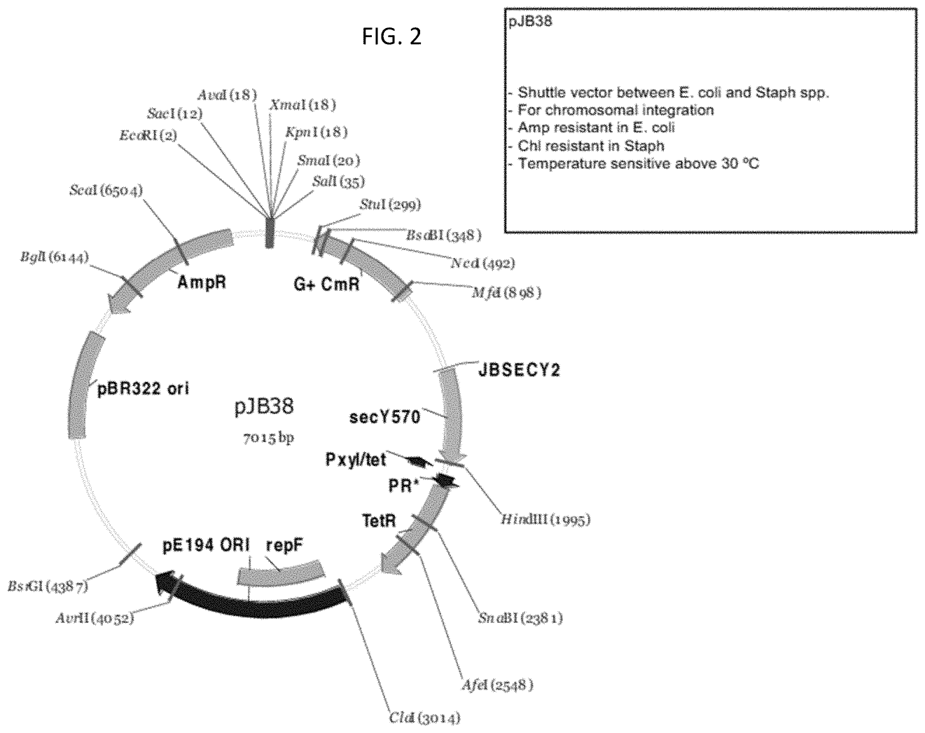

International Patent Application No. PCT/US2018/037850, incorporated by reference in its entirety herein, discloses various LEKTI recombinant proteins expressed by an engineered microbe. FIG. 1 from International Patent Application No. PCT/US2018/037850 shows a vector construct comprising the therapeutic LEKTI domains. The protein coding regions of the plasmid comprise SecA, 6×His tag (SEQ ID NO: 120), LEKTI D8-11, and RMR tag, operably linked to each other and under the control of a Cm^{R} promoter. FIG. 2 shows a vector construct of the pJB38 plasmid according to some embodiments of the present invention.

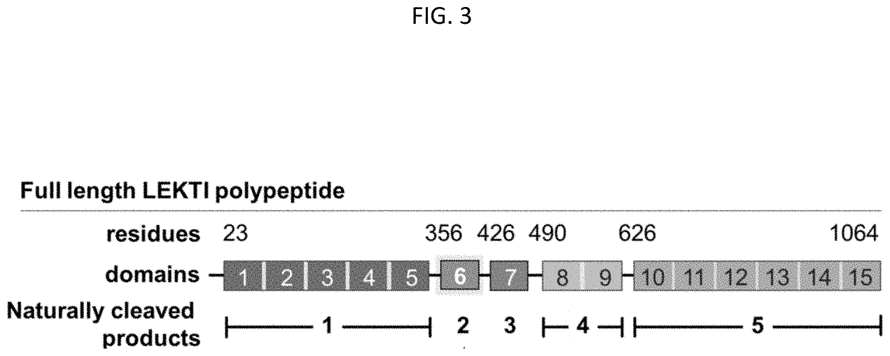

The LEKTI protein requires proteolytic cleavage for activation of its inhibitory function against many proteases. The full length protein is cleaved into domains D1-D5 and D6-D15. The D6-D15 domains are then further cleaved in multiple steps into D6-D9 and D10-D15, 4 D6 and D7-D9→D7 and D8-D9→D8. A schematic of the full-length LEKTI polypeptides, the domains and the naturally cleaved products is shown in FIG. 3 of International Patent Application No. PCT/US2018/037850, incorporated by reference in its entirety herein. The amino acid sequence of full length LEKTI protein is shown below as SEQ ID NO: 103. Each of the 15 individual LEKTI domains are shown below as SEQ ID NOs 104-118:

LEKTI amino acid sequence Residues 1-1064
(SEQ ID NO: 103):
MKIATVSVLLPLALCLIQDAASKNEDQEMCHEFQAFMKNGKLFCPQDKKFF
QSLDGIMFINKCATCKMILEKEAKSQKRARHLARAPKATAPTELNCDDFKK
GERDGDFICPDYYEAVCGTDGKTYDNRCALCAENAKTGSQIGVKSEGECKS
SNPEQDVCSAFRPFVRDGRLGCTRENDPVLGPDGKTHGNKCAMCAELFLKE
AENAKREGETRIRRNAEKDFCKEYEKQVRNGRLFCTRESDPVRGPDGRMHG
NKCALCAEIFKQRFSEENSKTDQNLGKAEEKTKVKREIVKLCSQYQNQAKN
GILFCTRENDPIRGPDGKMHGNLCSMCQAYFQAENEEKKKAEARARNKRES
GKATSYAELCSEYRKLVRNGKLACTRENDPIQGPDGKVHGNTCSMCEVFFQ
AEEEEKKKKEGKSRNKRQSKSTASFEELCSEYRKSRKNGRLFCTRENDPIQ
GPDGKMHGNTCSMCEAFFQQEERARAKAKREAAKEICSEFRDQVRNGTLIC
TREHNPVRGPDGKMHGNKCAMCASVFKLEEEEKKNDKEEKGKVEAEKVKRE
AVQELCSEYRHYVRNGRLPCTRENDPIEGLDGKIHGNTCSMCEAFFQQEAK
EKERAEPRAKVKREAEKETCDEFRRLLQNGKLFCTRENDPVRGPDGKTHGN
KCAMCKAVFQKENEERKRKEEEDQRNAAGHGSSGGGGGNTQDECAEYREQM
KNGRLSCTRESDPVRDADGKSYNNQCTMCKAKLEREAERKNEYSRSRSNGT
GSESGKDTCDEFRSQMKNGKLICTRESDPVRGPDGKTHGNKCTMCKEKLER
EAAEKKKKEDEDRSNTGERSNTGERSNDKEDLCREFRSMQRNGKLICTREN
NPVRGPYGKMHINKCAMCQSIFDREANERKKKDEEKSSSKPSNNAKDECSE
FRNYIRNNELICPRENDPVHGADGKFYTNKCYMCRAVFLTEALERAKLQEK
PSHVRASQEEDSPDSFSSLDSEMCKDYRVLPRIGYLCPKDLKPVCGDDGQT
YNNPCMLCHENLIRQTNTHIRSTGKCEESSTPGTTAASMPPSDE

LEKTI Domains (and residues corresponding to the numbering of SEQ ID NO: 103) are set forth below:

LEKTI Domain 1
(residues 23-77; SEQ ID NO: 104)
KNEDQEMCHEFQAFMKNGKLFCPQDKKFFQSLDGIMFINKCATCKMILEKE
AKSQ
LEKTI Domain 2
(residues 91-153; SEQ ID NO: 105)
APTELNCDDFKKGERDGDFICPDYYEAVCGTDGKTYDNRCALCAENAKTGS
QIGVKSEGECKS
LEKTI Domain 3
(residues 155-216; SEQ ID NO: 106)
NPEQDVCSAFRPFVRDGRLGCTRENDPVLGPDGKTHGNKCAMCAELFLKEA
ENAKREGETRI
LEKTI Domain 4
(residues 219-285; SEQ ID NO: 107)
NAEKDFCKEYEKQVRNGRLFCTRESDPVRGPDGRMHGNKCALCAEIFKQRF
SEENSKTDQNLGKAEE
LEKTI Domain 5
(residues 291-352; SEQ ID NO: 108)
REIVKLCSQYQNQAKNGILFCTRENDPIRGPDGKMHGNLCSMCQAYFQAEN
EEKKKAEARAR
LEKTI Domain 6
(residues 356-423; SEQ ID NO: 109)
ESGKATSYAELCSEYRKLVRNGKLACTRENDPIQGPDGKVHGNTCSMCEVF
FQAEEEEKKKKEGKSRN
LEKTI Domain 7
(residues 431-489; SEQ ID NO: 110)
ASFEELCSEYRKSRKNGRLFCTRENDPIQGPDGKMHGNTCSMCEAFFQQEE
RARAKAKR
LEKTI Domain 8
(residues 490-550; SEQ ID NO: 111)
EAAKEICSEFRDQVRNGTLICTREHNPVRGPDGKMHGNKCAMCASVFKLEE
EEKKNDKEEKG
LEKTI Domain 9
(residues 561622; SEQ ID NO: 112)
EAVQELCSEYRHYVRNGRLPCTRENDPIEGLDGKIHGNTCSMCEAFFQQEA
KEKERAEPRAK
LEKTI Domain 10
(residues 626-688; SEQ ID NO: 113)
EAEKETCDEFRRLLQNGKLFCTRENDPVRGPDGKTHGNKCAMCKAVFQKEN
EERKRKEEEDQR
LEKTI Domain 11
(residues 701-757; SEQ ID NO: 114)
GNTQDECAEYREQMKNGRLSCTRESDPVRDADGKSYNNQCTMCKAKLEREA
ERKNEY
LEKTI Domain 12
(residues 768-830; SEQ ID NO: 115)
ESGKDTCDEFRSQMKNGKLICTRESDPVRGPDGKTHGNKCTMCKEKLEREA
AEKKKKEDEDRS
LEKTI Domain 13
(residues 843-905; SEQ ID NO: 116)
NDKEDLCREFRSMQRNGKLICTRENNPVRGPYGKMHINKCAMCQSIFDREA
NERKKKDEEKSS
LEKTI Domain 14
(residues 910-970; SEQ ID NO: 117)
NNAKDECSEFRNYIRNNELICPRENDPVHGADGKFYTNKCYMCRAVFLTEA
LERAKLQEKPS
LEKTI Domain 15
(residues 987-1048; SEQ ID NO: 118)
SLDSEMCKDYRVLPRIGYLCPKDLKPVCGDDGQTYNNPCMLCHENLIRQTN
THIRSTGKCEE
LEKTI nucleic acid sequence is set forth below as
SEQ ID NO: 119.
LEKTI Full length Nucleic acid sequence
(SEQ ID NO: 119)
ATGAAGATAGCCACAGTGTCAGTGCTTCTGCCCTTGGCTCTTTGCCTCATA
CAAGATGCTGCCAGTAAGAATGAAGATCAGGAAATGTGCCATGAATTTCAG
GCATTTATGAAAAATGGAAAACTGTTCTGTCCCCAGGATAAGAAATTTTTT
CAAAGTCTTGATGGAATAATGTTCATCAATAAATGTGCCACGTGCAAAATG
ATACTGGAAAAAGAAGCAAAATCACAGAAGAGGGCCAGGCATTTAGCAAGA
GCTCCCAAGGCTACTGCCCCAACAGAGCTGAATTGTGATGATTTTAAAAAA
GGAGAAAGAGATGGGGATTTTATCTGTCCTGATTATTATGAAGCTGTTTGT
GGCACAGATGGGAAAACATATGACAACAGATGTGCACTGTGTGCTGAGAAT
GCGAAAACCGGGTCCCAAATTGGTGTAAAAAGTGAAGGGGAATGTAAGAGC
AGTAATCCAGAGCAGGATGTATGCAGTGCTTTTCGGCCCTTTGTTAGAGAT
GGAAGACTTGGATGCACAAGGGAAAATGATCCTGTTCTTGGTCCTGATGGG
AAGACGCATGGCAATAAGTGTGCAATGTGTGCTGAGCTGTTTTTAAAAGAA
GCTGAAAATGCCAAGCGAGAGGGTGAAACTAGAATTCGACGAAATGCTGAA
AAGGATTTTTGCAAGGAATATGAAAAACAAGTGAGAAATGGAAGGCTTTTT
TGTACACGGGAGAGTGATCCAGTCCGTGGCCCTGACGGCAGGATGCATGGC
AACAAATGTGCCCTGTGTGCTGAAATTTTCAAGCAGCGTTTTTCAGAGGAA
AACAGTAAAACAGATCAAAATTTGGGAAAAGCTGAAGAAAAAACTAAAGTT
AAAAGAGAAATTGTGAAACTCTGCAGTCAATATCAAAATCAGGCAAAGAAT
GGAATACTTTTCTGTACCAGAGAAAATGACCCTATTCGTGGTCCAGATGGG
AAAATGCATGGCAACTTGTGTTCCATGTGTCAAGCCTACTTCCAAGCAGAA
AATGAAGAAAAGAAAAAGGCTGAAGCACGAGCTAGAAACAAAAGAGAATCT
GGAAAAGCAACCTCATATGCAGAGCTTTGCAGTGAATATCGAAAGCTTGTG
AGGAACGGAAAACTTGCTTGCACCAGAGAGAACGATCCTATCCAGGGCCCA
GATGGGAAAGTGCATGGCAACACCTGCTCCATGTGTGAGGTCTTCTTCCAA
GCAGAAGAAGAAGAAAAGAAAAAGAAGGAAGGTAAATCAAGAAACAAAAGA
CAATCTAAGAGTACAGCTTCCTTTGAGGAGTTGTGTAGTGAATACCGCAAA
TCCAGGAAAAACGGACGGCTTTTTTGCACCAGAGAGAATGACCCCATCCAG
GGCCCAGATGGAAAAATGCATGGCAACACCTGCTCCATGTGTGAGGCCTTC
TTTCAACAAGAAGAAAGAGCAAGAGCAAAGGCTAAAAGAGAAGCTGCAAAG
GAAATCTGCAGTGAATTTCGGGACCAAGTGAGGAATGGAACACTTATATGC
ACCAGGGAGCATAATCCTGTCCGTGGCCCAGATGGCAAAATGCATGGAAAC
AAGTGTGCCATGTGTGCCAGTGTGTTCAAACTTGAAGAAGAAGAGAAGAAA
AATGATAAAGAAGAAAAAGGGAAAGTCGAGGCTGAAAAAGTTAAGAGAGAA
GCAGTTCAGGAGCTGTGCAGTGAATATCGTCATTATGTGAGGAATGGACGA
CTCCCCTGTACCAGAGAGAATGATCCTATTGAGGGTCTAGATGGGAAAATC
CACGGCAACACCTGCTCCATGTGTGAAGCCTTCTTCCAGCAAGAAGCAAAA
GAAAAAGAAAGAGCTGAACCCAGAGCAAAAGTCAAAAGAGAAGCTGAAAAG
GAGACATGCGATGAATTTCGGAGACTTTTGCAAAATGGAAAACTTTTCTGC
ACAAGAGAAAATGATCCTGTGCGTGGCCCAGATGGCAAGACCCATGGCAAC
AAGTGTGCCATGTGTAAGGCAGTCTTCCAGAAAGAAAATGAGGAAAGAAAG
AGGAAAGAAGAGGAAGATCAGAGAAATGCTGCAGGACATGGTTCCAGTGGT
GGTGGAGGAGGAAACACTCAGGACGAATGTGCTGAGTATCGGGAACAAATG
AAAAATGGAAGACTCAGCTGTACTCGGGAGAGTGATCCTGTACGTGATGCT
GATGGCAAATCGTACAACAATCAGTGTACCATGTGTAAAGCAAAATTGGAA
AGAGAAGCAGAGAGAAAAAATGAGTATTCTCGCTCCAGATCAAATGGGACT
GGATCAGAATCAGGGAAGGATACATGTGATGAGTTTAGAAGCCAAATGAAA
AATGGAAAACTCATCTGCACTCGAGAAAGTGACCCTGTCCGGGGTCCAGAT
GGCAAGACACATGGCAATAAGTGTACTATGTGTAAGGAAAAACTGGAAAGG
GAAGCAGCTGAAAAAAAAAAGAAAGAGGATGAAGACAGGAGCAATACAGGA
GAAAGGAGCAATACAGGAGAAAGGAGCAATGACAAAGAGGATCTGTGTCGT
GAATTTCGAAGCATGCAGAGAAATGGAAAGCTTATCTGCACCAGAGAAAAT
AACCCTGTTCGAGGCCCATATGGCAAGATGCACATCAATAAATGTGCTATG
TGTCAGAGCATCTTTGATCGAGAAGCTAATGAAAGAAAAAAGAAAGATGAA
GAGAAATCAAGTAGCAAGCCCTCAAATAATGCAAAGGATGAGTGCAGTGAA
TTTCGAAACTATATAAGGAACAATGAACTCATCTGCCCTAGAGAGAATGAC
CCAGTGCACGGTGCTGATGGAAAGTTCTATACAAACAAGTGCTACATGTGC
AGAGCTGTCTTTCTAACAGAAGCTTTGGAAAGGGCAAAGCTTCAAGAAAAG
CCATCCCATGTTAGAGCTTCTCAAGAGGAAGACAGCCCAGACTCTTTCAGT
TCTCTGGATTCTGAGATGTGCAAAGACTACCGAGTATTGCCCAGGATAGGT
TATCTTTGTCCAAAGGATTTAAAGCCTGTCTGTGGTGACGATGGCCAAACC
TACAACAATCCTTGCATGCTCTGTCATGAAAACCTGATACGCCAAACAAAT
ACACACATCCGCAGTACAGGGAAGTGTGAGGAGAGCAGCACCCCAGGAACC
ACCGCAGCCAGCATGCCCCCGTCTGACGAA
LEKTI domain 6 (D6) nucleic acid sequence
(SEQ ID NO: 128)
gaatctggtaaagcaacgagttacgctgaattgtgctcagaataccgtaag
ttagtcagaaacggaaagttggcgtgtactcgtgagaacgaccctatccag
ggaccggacggcaaggtacatggaaatacttgcagtatgtgcgaggtgata
ccaggcggaggaagaagaaaagaagaaaaaggagggcaaaagtcgaaatTA
A

According to some embodiments, the disclosure relates to the full length LEKTI molecule, one or more of domains D1, D2, D3, D4, D5, D6, D7, D8, D9, D10, D11, D12, D13, D14, and D15, as well as isolated fragments, oligonucleotides, and truncations maintaining biological activity, for example N-terminal deletions, C-terminal deletions, or deletions at both N and C-termini derived from SEQ ID NO: 119 and deduced amino acid sequence SEQ ID NO: 103. The amino acid sequence of full length LEKTI protein is set forth as SEQ ID NO: 103, as well as each of the 15 individual domains as shown below.

The present disclosure also relates to allelic variants of LEKTI, or portions thereof (one or more of domains D1, D2, D3, D4, D5, D6, D7, D8, D9, D10, D11, D12, D13, D14, and D15), as well as synthetic or mutated genes of SPINK (e.g., SPINK5) that have been modified to change, for example, the expression or activity of the recombinant protein. It is also noted that degeneracy of the nucleic acid code can be considered variations in the nucleotide sequences that encode the same amino acid residues. Accordingly, the disclosure includes nucleic acid residues that are able to hybridize under moderately stringent conditions. One skilled in the art can determine effective combinations of salt and temperature to constitute a moderately stringent hybridization condition. It is also envisioned that orthologs of SPINK genes are present in other species, for example, dog, sheep, rat, hamster, chicken and pig. Therefore in another embodiment of the present invention relates to SPINK (e.g., SPINK5) nucleic acids that encode polypeptides having at least about 70% to 80% identity, preferably 90% to 95% identity (e.g., 90%, 91%, 92%, 93%, 94%, 95%), more preferably 98% to 99% identity to LEKTI set forth in SEQ ID NO: 103 or portions thereof (one or more of domains D1, D2, D3, D4, D5, D6, D7, D8, D9, D10, D11, D12, D13, D14, and D15).

The present disclosure also provides recombinant vectors containing a nucleotide sequence encoding SEQ ID NO: 103 or portions thereof (one or more of domains D1, D2, D3, D4, D5, D6, D7, D8, D9, D10, D11, D12, D13, D14, and D15). Recombinant vectors include but are not limited to vectors useful for the expression of the open reading frames (ORFs) in E. coli, yeast, viral, baculovirus, plants or plant cells, as well as mammalian cells.

Expression Systems Useful for Production of LEKTI or Portions Thereof

The present disclosure also provides for recombinant cloning and expression vectors useful for the production of biologically active LEKTI. Such expression plasmids may be used to prepare recombinant LEKTI polypeptides or portions thereof (one or more of domains D1, D2, D3, D4, D5, D6, D7, D8, D9, D10, D11, D12, D13, D14, and D15) encoded by the nucleic acids in a suitable host organism. Suitable host organisms for the production of LEKTI or portions thereof (one or more of domains D1, D2, D3, D4, D5, D6, D7, D8, D9, D10, D11, D12, D13, D14, and D15) include, but are not limited to bacteria, yeast, insect cells, mammalian cells, plants and plant cells. In addition, cell free systems may also be employed for the production of recombinant proteins. One skilled in the art can readily prepare plasmids suitable for the expression of recombinant LEKTI in the suitable host organism. Appropriate cloning and expression vectors are readily available to one skilled in the art and can be obtained from commercial sources or from the ATCC.

The recombinant protein can be produced in the within the host cell or secreted into the culture medium depending on the nature of the vector system used for the production of the recombinant protein. Generally plasmids useful for the expression of the recombinant LEKTI or portions thereof (one or more of domains D1, D2, D3, D4, D5, D6, D7, D8, D9, D10, D11, D12, D13, D14, and D15) comprise necessary operable linked regulatory elements such as a promoter sequence (including operators, enhancers, silencers, ribosomal binding sites), transcriptional enhancing sequences, translational fusions to signal peptides (native or heterologous) or peptide sequences useful for the purification of recombinant protein (for example His Tag, FLAG® (a convenient binding moiety), MBP, GST), transcription termination signals and poly adenylation signals (if necessary).

It may also be necessary for the recombinant plasmid to replicate in the host cell. This requires the use of an origin of replication suitable for the host organism. Alternatively, the recombinant expression plasmid may be stably integrated into the host's chromosome. This may require homologous recombination or random integration into the host chromosomes. Both instances require the use of an appropriate selection mechanism to distinguish transformed host cells from non-transformed host cells. Useful selection schemes include the use of, for example, antibiotics (for example, G418, ZEOCIN® (a glycopeptide antibiotic of the bleomycin family), kanamycin, tetracycline, gentamycin, spectinomycin, ampicillin), complementation of an auxotroph (for example Trp−, DHFR−), and scorable markers (for example β-glucoronidase, β-galactosidase, GFP).

Expression systems useful in the present invention include yeast systems. Plasmid vectors particularly useful for the transformation and expression of protein in recombinant K lactis have been descried (Chen, X-J., Gene (1996) 172:131-136). Other yeast expression systems based on Saccharomyces cerevisiae or Pichia pastoris or Pichia methanolica may also be useful for the recombinant production of LEKTI or portions thereof (one or more of domains D1, D2, D3, D4, D5, D6, D7, D8, D9, D10, D11, D12, D13, D14, and D15). Expression plasmid suitable for the expression of LEKTI or portions thereof (one or more of domains D1, D2, D3, D4, D5, D6, D7, D8, D9, D10, D11, D12, D13, D14, and D15) in S. cerevisiae, P. pastoris, or P. methanolica may be obtained from a commercial source or ATCC. Plasmids described above may also be modified by one skilled in the art to optimize, for example, promoter sequences and or secretion signals optimal for the host organism and recombinant production of LEKTI. Established methods are also available to one skilled in the art for introducing recombinant plasmid into the yeast strains.

Expression of recombinant LEKTI or portions thereof (one or more of domains D1, D2, D3, D4, D5, D6, D7, D8, D9, D10, D11, D12, D13, D14, and D15) in mammalian cell culture is also a preferred embodiment of the present invention. There are a wide variety of mammalian cell lines available to one skilled in the art. The most widely used and most successful mammalian expression system is based on a dhfr− (dihydrofolate reductase) Chinese hamster ovary (CHO) cell line along with a suitable expression plasmid containing the dhfr gene and suitable promoter sequence. The cells may be transfected for transient expression or stable expression of the protein of interest. Other factors for the production of foreign protein in mammalian cells including regulatory considerations have been reviewed (Bendig, M., Genetic Engineering (1988) 7:91-127). One useful mammalian expression system is based on the EF-1α promoter (Mizushima, S and Nagata Nucleic Acids Res (1990) 18:5322) and Human embryonic kidney (EK) 293T cell line (Chen, P., et al., Protein Expression and Purification (2002) 24:481-488). Variants of the commercially available CHO and 293T cells lines and their suitable growth and expression media may be used to further improve protein production yields. Variants of commercially available expression vectors including different promoters, secretion signals, transcription enhancers, etc., may also be used to improve protein production yields.

Another useful expression system includes expression in E. coli. There are several expression systems known to one skilled in the art for production of recombinant proteins in E. coli. Expression of mammalian protein in E. coli has not been particularly useful due to the fact that many mammalian proteins are post translationally modified by glycosylation or may contain intra or inter di-sulfide molecular bonds. Particular E. coli expression plasmid useful in the present invention may include, for example, fusions with signal peptides to target the protein to the periplasmic space. Additionally, E. coli host strains that contain mutations in both the thioredoxin reductase (trxB) and glutathione reductase (gor) genes greatly enhance disulfide bond formation in the cytoplasm (Prinz, W. A., et al., J. Biol. Chem. (1997) 272:15661-15667). The addition of thioredoxin fused to the N-terminus or C-terminus of LEKTI may also aid in the production of soluble protein in E. coli cells. (LaVallie, E. R., et al., Bio/Technology (1993) 11:187-193).

Other expression systems known in the art may also be employed for the production of LEKTI or portions thereof (one or more of domains D1, D2, D3, D4, D5, D6, D7, D8, D9, D10, D11, D12, D13, D14, and D15), and include but are not limited to, baculovirus expression (Luckow V., Curr Opin Biotechnol (1993) 5:564-572).

LEKTI or portions thereof (one or more of domains D1, D2, D3, D4, D5, D6, D7, D8, D9, D10, D11, D12, D13, D14, and D15) may be purified from the recombinant expression system using techniques known to one normally skilled in the art. Expression of the LEKTI protein or portions thereof (one or more of domains D1, D2, D3, D4, D5, D6, D7, D8, D9, D10, D11, D12, D13, D14, and D15) can either be intracellular or secreted in the media fraction. Secretion of LEKTI into the media simplifies protein purification. Expression of intracellular LEKTI or portions thereof (one or more of domains D1, D2, D3, D4, D5, D6, D7, D8, D9, D10, D11, D12, D13, D14, and D15) requires disruption of the cell pellets by any convenient method including freeze-thaw, mechanical disruption, sonication, or use of detergents or cell lysing enzymes or agents. Following disruption or concentration of secreted protein, purification can be accomplished by a number of methods know to one skilled in the art. For example, commercially available affinity chromatography may be used to purify recombinant LEKTI or portions thereof (one or more of domains D1, D2, D3, D4, D5, D6, D7, D8, D9, D10, D11, D12, D13, D14, and D15) fused with affinity tags such as: 6×HIS (SEQ ID NO: 120), FLAG® (a convenient binding moiety), GST, or MBP. In addition, antibodies specific to LEKTI or portions thereof (one or more of domains D1, D2, D3, D4, D5, D6, D7, D8, D9, D10, D11, D12, D13, D14, and D15) may be used for affinity purification. In addition, matrices chemically modified with a ligand having strong affinity to LEKTI or portions thereof (one or more of domains D1, D2, D3, D4, D5, D6, D7, D8, D9, D10, D11, D12, D13, D14, and D15) as a substrate mimic may also be used for affinity purification. LEKTI may also be purified with the use of an affinity tag or antibodies following conventional protein purification methods know to one skilled in the art.

According to some embodiments, non-viral gene delivery can also be used. Examples include diffusion of DNA in the absence of any carriers or stabilizers (“naked DNA”), DNA in the presence of pharmacologic stabilizers or carriers (“formulated DNA”), DNA complexed to proteins that facilitate entry into the cell (“Molecular conjugates”), or DNA complexed to lipids.

According to some embodiments, the disclosure provides microbial compositions comprising one or more of a wide range of bacteria. Examples include, but are not limited to, non-pathogenic and commensal bacteria. Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp. According to some embodiments, bacteria in the microbial compositions comprise one or more of Staphylococcus epidermidis, Staphylococcus hominis, Staphylococcus warneri, Streptococcus pyogenes, Streptococcus mitis, Lactobacillus acidophilus, Propionibacterium acnes, Acinetobacter johnsonii, and Pseudomonas aeruginosa and mixtures thereof.

According to some embodiments of any one of the above aspects, the microbe is a Staphylococcus spp. According to some embodiments, other related or similar species found on the skin are used.

According to some embodiments, the microbe is engineered to express a mammalian gene encoding LEKTI protein.

According to some embodiments, apathogenic anaerobic bacteria are used to selectively deliver foreign genes into tumor cells. For example, it has been shown that Clostridium acetobutylicum spores injected intravenously into mice bearing tumors, germinated only in the necrotic areas of tumors that had low oxygen tension. Thus, an apathogenic anaerobic bacteria can be used to selectively express LEKTI or portions thereof (one or more of domains D1, D2, D3, D4, D5, D6, D7, D8, D9, D10, D11, D12, D13, D14, and D15) in tumor masses with necrotic, anaerobic centers.

Certain embodiments involve the use of bacterium Staphylococcus epidermidis. According to some embodiments, the strain of S. epidermidis to be used is incapable of producing biofilms. An example of this is S. epidermidis strain American Type Culture Collection (ATCC) 12228 or ARS Culture Collection (NRRL) B-4268.

Recent research has shown that the microbiome plays an important role in maintaining and influencing key physiologic activities, including normal metabolism and immune function. Laboratory and clinical evidence suggest that changes in specific organisms in the microbiome may lead to the development of disease, including cancer. Microorganisms cause an estimated 20% of human cancer. The best-known example is the role Helicobater pylori plays in gastric cancer. DNA and RNA viruses have also been identified as etiological factors in cervical, oropharyngeal, nasopharyngeal, and hepatocellular carcinoma. A number of studies have reported links between the human microbiome, immunomodulators, inflammation and tumor initiation or progression in oral, colon, pancreatic, liver, esophageal and prostate cancers. There are several mechanisms by which bacterial infection can lead to the initiation and progression of oncogenic processes. Bacterial endotoxins, metabolic byproducts of bacterial infection, and increased enzymatic activity as a result of bacterial infection, can induce somatic mutations and signaling pathway alterations. The role of microbes and viruses in cancer development is also associated to a wide spectrum of focalized changes driven by innate and adaptive immune responses. Harnessing the host immune system by microbiome modulation constitutes a promising approach for the treatment of cancer because of its potential to specifically target tumor cells while limiting harm to normal tissue, with durability of benefit associated with immunologic memory.

Accordingly, in some embodiments, the disclosure provides microbial compositions comprising a microbe that is genetically modified to express one or more LEKTI protein domains encoded by one or more SPINK genes. According to some embodiments, the one or more SPINK genes encodes one or more LEKTI proteins, or portions thereof (e.g., one or more of domains D1, D2, D3, D4, D5, D6, D7, D8, D9, D10, D11, D12, D13, D14, and D15) that are used to modify the tumor microbiome.

According to some embodiments, the recombinant microbe is adapted to live indefinitely or for a controlled duration on an epithelial or mucosal surface of a mammal to provide a continuous supply of LEKTI protein domains. According to some embodiments, the continuous supply of LEKTI protein domain is provided by constitutively expressed LEKTI. According to some embodiments, the continuous supply of LEKTI protein domain is provided by LEKTI that is inducibly expressed. According to some embodiments, the recombinant microbe lives alongside commensal microorganisms naturally occurring on an epithelial or mucosal surface of the mammal According to some embodiments, the recombinant microbe lives to the exclusion of commensal microorganisms that naturally occur on an epithelial or mucosal surface of the mammal According to some embodiments, the recombinant microbe is adapted to multiply on an epithelial or mucosal surface of the mammal. According to some embodiments, the recombinant microbe is no longer alive, but contains effective amounts of a therapeutic polypeptide, e.g. LEKTI or therapeutically effective domain(s) thereof. Such cells may be intact or not depending upon the particulars of administering the therapeutic peptide (or domain(s) thereof) to the target site.

According to some embodiments, the microbe is selected from the group consisting of Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp. According to some embodiments, bacteria in the microbial compositions comprise one or more of Staphylococcus epidermidis, Staphylococcus hominis, Staphylococcus warneri, Streptococcus pyogenes, Streptococcus mitis, Lactobacillus acidophilus, Propionibacterium acnes, Acinetobacter johnsonii, and Pseudomonas aeruginosa and mixtures thereof.

According to some embodiments of any one of the above aspects, the microbe is a Staphylococcus spp. According to some embodiments, the microbe is Staphylococcus epidermidis.

According to some embodiments, the LEKTI protein (or domains thereof) is recombinantly produced and administered. According to some embodiments, the LEKTI protein (or domains thereof) is administered in a composition not including a microbe.

According to some embodiments, the recombinant protein expressed by the engineered microbe comprises the peptide sequence according to SEQ ID NO: 109. In a specific embodiment, the LEKTI domain is Domain 6 (LEKTI D6).

According to some embodiments, the recombinant protein expressed by the engineered microbe comprises a peptide sequence selected from any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 85% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 90% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 95% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 96% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 97% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 98% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe comprises a peptide sequence that is at least 99% identical to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118. According to some embodiments, the recombinant protein expressed by the engineered microbe consists of any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118.

According to some embodiments, the recombinant microbe comprises a sequence as disclosed herein that has at least about 75% identity, 80% identity, 85% identity, 90% identity, 95% identity, 96% identity, 97% identity, 98% identity, or 99% identity to any one or more of the SEQ ID NOS listed herein. As used herein, the term “identity” and grammatical versions thereof means the extent to which two nucleotide or amino acid sequences have the same residues at the same positions in an alignment. Percent (%) identity is calculated by multiplying the number of matches in a sequence alignment by 100 and dividing by the length of the aligned region, including internal gaps.

According to some embodiments, the recombinant protein expressed by the engineered microbe comprises one or more protease inhibitory domains of the LEKTI protein. Some non-limiting examples include one or more of domains D1, D2, D3, D4, D5, D6, D7, D8, D9, D10, D11, D12, D13, D14, and D15. According to some embodiments, the recombinant protein expressed by the engineered microbe comprises LEKTI inhibitory domain 6 (D6) or domains D8 to D11.

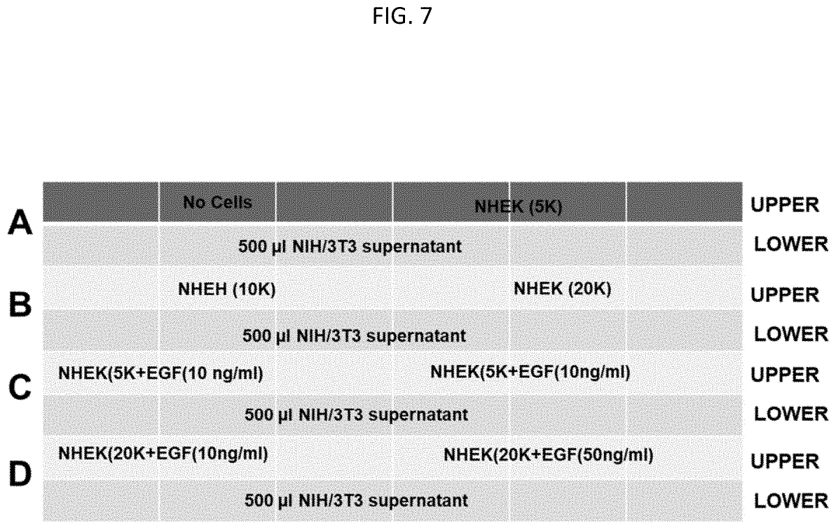

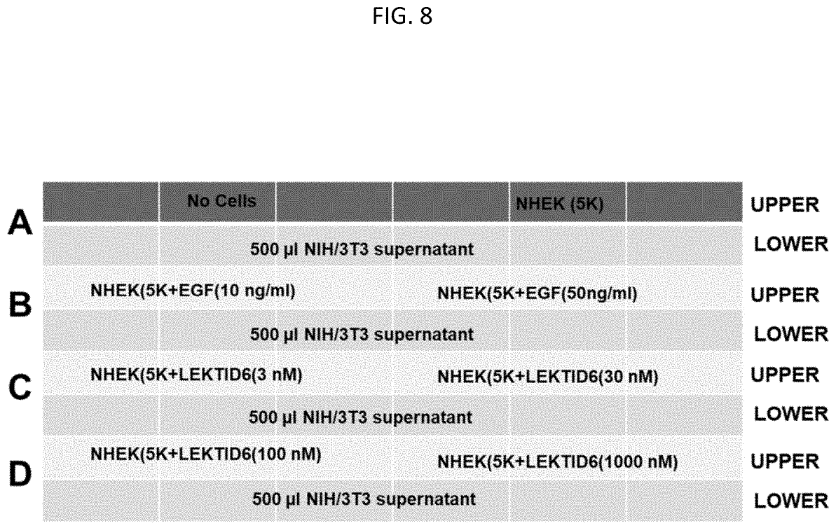

International Patent Application No. PCT/US2018/037850, incorporated by reference in its entirety herein, examines the capacity of purified recombinant LEKTI Domain 6 (LEKTI D6) fragments to function in vitro as a serine protease inhibitor. FIG. 7A and FIG. 7B from PCT/US2018/037850 show recombinantly produced LEKTI Domain 6 inhibits trypsin in vitro. FIG. 8A and FIG. 8B from PCT/US2018/037850 show recombinantly produced LEKTI Domain 6 (with His6 tag (SEQ ID NO: 120)) inhibits trypsin in vitro compared to LEKTI domains 10-15. FIG. 9A and FIG. 9B from PCT/US2018/037850 show recombinantly produced LEKTI Domain 6 inhibits KLK7 in vitro similar to inhibition of KLK7 by LEKTI domains 10-15.

According to some embodiments, the LEKTI protein domains are effective to treat, prevent or delay the progression of cancer in a subject. According to some embodiments, the LEKTI protein domains act as a competitive or non-competitive inhibitor of one or more proteases present on or in the skin of a mammal According to some embodiments, the LEKTI protein domain acts as a serine protease inhibitor.

Secretion Peptides

According to some embodiments, the therapeutic LEKTI domain is operably linked to one or more secretion signals or export signals that tag the protein for transport through the secretory pathway. According to some embodiments, the secretory peptide may be positioned on the N-terminal end of a recombinant protein, and may co-translationally or post-translationally target the tagged protein for secretion. According to some embodiments, at least one LEKTI domain is operably linked to a SecA domain. Any secretion signal that facilitates exit of the LEKTI protein out of the bacterial cell may be used as a secretion peptide. Non-limiting examples of secretion peptides signals are set forth in Table 1, below:

TABLE 1

| Amino Acid Sequence | SEQ ID NO: |
|---|---|
| MKKLAFAITAASGAAAVLSHHDAEA | 9 |
| WLDNRAFSKKFVPVVMATSVALFFLNLAFA | 10 |
| MAKKFNYKLPSMVALTLFGTAFTAHQANA | 11 |
| MKKRFLSICTMTIAALATTTMVNTSYA | 12 |
| NLKKQSKLILIFICIFTFFIMIIQSQFLMG | 13 |
| MKIFKLTSLTLAALTLAFPFSHVAQA | 14 |
| MKKTVIASTLAVSLGIAGYGLSGHEAH | 15 |
| MKKNKFLVYLLSTALITPTFATQTAFA | 16 |
| MKTRQNKYSIRKFSVGASSILIAALLFMGGGSAQA | 17 |
| MKNNNETRRFSIRKYTVGVVSIITGITIFVSGQHAQA | 18 |
| MKKKLSYMITIMLAFTLSLALGLFFNSAHA | 19 |

According to some embodiments, the therapeutic LEKTI domain is operably linked to one or more signal sequences derived from endogenous proteins of Staphylococcus epidermidis. Non-limiting examples of secretion signal peptides derived from endogenous proteins of Staphylococcus epidermidis are set forth in Table 2 below:

TABLE 2

| Protein Name | Amino Acid Length | Signal Sequence | SEQ ID NO |
|---|---|---|---|
| Serine-aspartate repeat- | 45 | MKKRRQGPINKRVDFLSNKVNKY | 20 |
| containing protein F | | SIRKFTVGTASILVGATLMFGA | |
| Glutamyl | 27 | MKKRFLSICTMTIAALATTTMVNT | 21 |
| endopeptidase | | SYA | |
| Bifunctional autolysin | 29 | MAKKFNYKLPSMVALTLFGTAFT | 22 |
| | | AHQANA | |
| Serine-aspartate repeat- | 50 | MIKKNNLLTKKKPIANKSNKYAIR | 23 |
| containing protein G | | KFTVGTASIVIGAALLFGLGHNEA | |
| | | KA | |
| Biofilm PIA synthesis | 30 | MKPFKLIFISALMILIMTNATPISHL | 24 |
| deacetylase icaB | | NAQA | |
| Lipase | 35 | MKTRQNKYSIRKFSVGASSILIAAL | 25 |
| | | LFMGGGSAQA | |
| Epidermin leader | 23 | MNKFKFFIVFLILSLVFLQNEYA | 26 |
| peptide-processing | | | |
| serine protease epiP | | | |
| Fibrinogen-binding | 51 | MINKKNNLLTKKKPIANKSNKYAI | 27 |
| protein | | RKFTVGTASIVIGATLLFGLGHNE | |
| | | AK | |
| | | A | |
| Staphylococcal | 26 | MKKIATATIATAGIATFAFAHHDA | 28 |
| secretory antigen ssaA | | QA | |
| Extracellular elastase | 28 | MKNFSKFALTSIAALTVASPLVNT | 29 |
| | | EVDA | |
| n/a | 37 | MKNNNETRRFSIRKYTVGVVSIIT | 30 |
| | | GITIFVSGQHAQA | |
| Uncharacterized | 19 | MRYLKRITIYISLLILVSG | 31 |
| lipoprotein SE_0145 | | | |
| Foldase protein prsA | 20 | MKLMNKIIVPVTASALLLGA | 32 |
| Probable cell wall | 40 | MKKIDSWLTKHGLKNRLTLVVIVI | 33 |
| amidase lytH | | FIIFLILLFMFVNLSD | |
| Membrane protein | 19 | MKKKALLPLFLGIMIFLAG | 34 |
| oxaA 2 | | | |
| Probable | 28 | MKKTVIASTLAVSLGIAGYGLSGH | 35 |
| transglycosylase isaA | | EAHA | |
| Probable quinol oxidase | 19 | MSKFKSLLLLFGTLILLSG | 36 |
| subunit 2 | | | |
| Probable | 27 | MKKTLVASSLAIGLGVVAGNAGH | 37 |
| transglycosylase sceD | | DAHA | |
| Bifunctional autolysin | 29 | MAKKFNYKLPSMVALTLFGTAFT | 38 |
| | | AHQANA | |
| Extracellular cysteine | 30 | MKKKLSYMITIMLAFTLSLALGLF | 39 |
| protease | | FNSAHA | |
| Membrane protein | 18 | MHKRLFITLLGFIILLAG | 40 |
| oxaA 1 | | | |
| Uncharacterized | 19 | MRYLKRITIYISLLILVSG | 41 |
| lipoprotein SE_0144 | | | |
| N-acetylmuramoyl-L- | 25 | MQKKYITAIIGTTALSALASTHAQ | 42 |
| alanine amidase slel | | A | |
| Uncharacterized | 22 | MKHSSKIIVFVSFLILTIFIGG | 43 |
| lipoprotein SE_0142 | | | |
| Phosphate-binding | 20 | MKKWQLVGTTVLGASVLLGA | 44 |
| protein pstS | | | |
| Accumulation- | 52 | MGKRRQGPINKKVDFLPNKLNKY | 45 |
| associated protein | | SIRKFTVGTASILLGSTLIFGSSHE | |
| | | AKA | |
| Staphylococcal | 26 | MKKIATATIATAGIATFAFAHHDA | 46 |
| secretory antigen ssaA | | QA | |
| Serine-aspartate repeat- | 45 | MKKRRQGPINKRVDFLSNKVNKY | 47 |
| containing protein F | | SIRKFTVGTASILVGATLMFGA | |
| Glutamyl | 27 | MKKRFLSICTMTIAALATTTMVNT | 48 |
| endopeptidase | | SYA | |
| Lipase | 35 | MKTRQNKYSIRKFSVGASSILIAAL | 49 |
| | | LFMGGGSAQA | |
| Extracellular elastase | 28 | MKNFSKFALTSIAALTVASPLVNT | 50 |
| | | EVDA | |
| Uncharacterized | 17 | MKKVLASATILSLMLVG | 51 |
| lipoprotein SE_1947 | | | |
| Uncharacterized | 22 | MKYYGKCISYISILILTFFIGG | 52 |
| lipoprotein | | | |
| SE_0186/SE_0187 | | | |
| Uncharacterized | 22 | MKHSSKIIVFVSFLILTIFIGG | 53 |
| lipoprotein SERP2423 | | | |
| Biofilm PIA synthesis | 30 | MKPFKLIFISALMILIMTNATPISHL | 54 |
| deacetylase icaB | | NAQA | |
| Probable quinol oxidase | 10 | MSKFKSLLLLFGTLILLSG | 55 |
| subunit 2 | | | |
| Probable | 27 | MKKTLVASSLAIGLGVVAGNAGH | 56 |
| transglycosylase sceD | | DAHA | |
| Uncharacterized | 19 | MHYLKKVTIYISLLILVSG | 57 |
| lipoprotein SERP2447 | | | |
| N-acetylmuramoyl-L- | 25 | MQKKYITAIIGTTALSALASTHAQ | 58 |
| alanine amidase slel | | A | |
| Uncharacterized | 22 | MKHSKKLLLCISFLLITFFIGG | 59 |
| lipoprotein SERP2445 | | | |
| Staphylococcal | 26 | MKKIATATIATAGIATFAFAHHDA | 60 |
| secretory antigen ssaA | | QA | |
| Uncharacterized | 19 | MRYLKKVTIYISLLILVSG | 61 |
| lipoprotein SERP2443 | | | |
| Glutamyl | 27 | MKKRFLSICTMTIAALATTTMVNT | 62 |
| endopeptidase | | SYA | |
| Phosphate-binding | 20 | MKKWQLVGTTVLGASVLLGA | 63 |
| protein pstS | | | |
| Bifunctional autolysin | 29 | MAKKFNYKLPSMVALTLFGTAFT | 64 |
| | | AHQANA | |
| Extracellular cysteine | 30 | MKKKLSYMITIMLAFTLSLALGLF | 65 |
| protease | | FNSAHA | |
| Membrane protein | 18 | MHKRLFITLLGFIILLAG | 66 |
| oxaA 1 | | | |
| Uncharacterized | 22 | MRYLKKVTIYISLLILTIFIGG | 67 |
| lipoprotein SERP2422 | | | |
| Uncharacterized | 17 | MKKVLASATILSLMLVG | 68 |
| lipoprotein SERP1959 | | | |
| Uncharacterized | 22 | MKHSKKLLLCISFLLITVFISG | 69 |
| lipoprotein SERP2453 | | | |
| Uncharacterized | 22 | MKHSKKLLLCISFLLITFFISG | 70 |
| lipoprotein SERP2465 | | | |
| Probable | 28 | MKKTVIASTLAVSLGIAGYGLSGH | 71 |
| transglycosylase isaA | | EAHA | |
| Uncharacterized | 22 | MKHSKKLLLCISFLLITIFISG | 72 |
| lipoprotein SERP2451 | | | |
| Probable cell wall | 40 | MKKIDSWLTKHGLKNRLTLVVIVI | 73 |
| amidase lytH | | FIIFLILLFMFVNLSD | |
| Membrane protein | 19 | MKKKALLPLFLGIMIFLAG | 74 |
| oxaA 2 | | | |
| Foldase protein prsA | 20 | MKLMNKIIVPVTASALLLGA | 75 |
| Lipase | 35 | MKTRQNKYSIRKFSVGASSILIAAL | 76 |
| | | LFMGGGSAQA | |

According to some embodiments, the therapeutic LEKTI domain is operably linked to one or more secretion signal sequences derived from endogenous proteins of other bacteria. Signal peptides derived from endogenous proteins of various bacteria are known in the art.

Cell Penetration Peptides

According to some embodiments, one or more cell penetrating peptides are used to mediate delivery of therapeutic proteins in vivo without using cell surface receptors and without causing significant membrane damage. According to some embodiments, the recombinant LEKTI domain is operably linked to a cell penetration peptide sequence that enhances the ability of the LEKTI domain to pass through a cell membrane. The term “enhance” as used to describe the cell penetration peptide/LEKTI, means that the cell penetration sequence improves the passage of recombinant LEKTI domain through a cell membrane relative to a recombinant LEKTI domain lacking the cell penetration sequence.

According to some embodiments, one or more cell penetrating peptides are operably linked to therapeutic proteins to facilitate entry into skin cells (e.g. keratinocytes). Non-limiting examples are set forth in Table 3, below:

TABLE 3

| Cell penetrating sequence | SEQ ID NO |
|---|---|
| GRKKRRQRRRPPQ | 77 |
| GWTLNS AGYLLGKINLKALAALAKKIL | 78 |
| KLALKLALKALKAALKLA | 79 |
| WEAKLAKALAKALAKHLAKALAKALKACEA | 80 |
| KETWWETWWTEWSQPKKKRKV | 81 |
| RRRRRRRRR | 82 |
| LGTYTQDFNKFHTFPQTAIGVGAP | 83 |
| RQIKWFQNRRMKWKK | 84 |
| YGRKKRRQRRR | 85 |
| RGGRLSYSRRRFSTSTGR | 86 |
| RRLSYSRRRF | 87 |
| PIRRRKKLRRLK | 88 |
| RRQRRTSKLMKR | 89 |
| RRRRNRTRRNRRRVR | 90 |
| KMTRAQRRAAARRNRWTAR | 91 |
| TRRQRTRRARRNR | 92 |
| GRKKRRQRRRPPQ | 93 |
| GRRRRRRRRRPPQ | 94 |
| GWTLNSAGYLLGKINLKALAALAKKIL | 95 |
| KLALKLALKLALALKLA | 96 |
| MGLGLHLLVLAAALQGAWSQPKKKRKV | 97 |
| GALFLGWLGAAGSTMGAWSQPKKKRKV | 98 |
| GALFLGFLGAAGSTMGAWSQPKKKRKV | 99 |
| GALFLGFLGAAGSTMGAWSQPKSKRKV | 100 |
| KETWWETWWTEWSQPKKKRKV | 101 |
| KETWFETWFTEWSQPKKKRKV | 102 |

According to some embodiments, cell penetrating peptides comprise periodic amino acid sequences. Non-limiting examples of periodic cell penetrating sequences include: Polyarginines, R×n (wherein 4<n<17) (SEQ ID NO: 126); Polylysines, K×n (wherein 4<n<17) (SEQ ID NO: 127); arginine repeats interspaced with 6-aminocaprotic acid residues (RAca), wherein there are 2 to 6 arginine repeats; arginine repeats interspaced with 4-aminobutyric acid (RAbu), wherein there are 2 to 6 arginine repeats; arginine repeats interspaced with methionine, wherein there are 2 to 6 arginine repeats; arginine repeats interspaced with threonine, wherein there are 2 to 6 arginine repeats; arginine repeats interspaced with serine, wherein there are 2 to 6 arginine repeats; and arginine repeats interspaced with alanine, wherein there are 2 to 6 arginine repeats.

According to some embodiments, the LEKTI domain is operably linked to an RMR domain.

According to some embodiments, expression of the LEKTI domain is controlled by an operon and the amount of LEKTI provided to the mammal's skin is proportional to the availability of an extrinsic factor. For example, according to some embodiments the recombinant LEKTI gene may be under the control of a xylose inducible promoter (e.g. xylose repressor (xylR), xylose operator (xylO), xylose isomerase gene (xylA) including the cis-acting catabolite-responsive element (CRE)), and the amount of recombinant LEKTI protein made available to the skin of the mammal controlled by the amount of exogenous xylose available to the recombinant microbe. According to some embodiments, the expression of the LEKTI domain is controlled by a promoter that is constitutively active. According to some embodiments, the expression of the LEKTI domain is controlled by a Cm^{R} promoter.

According to some embodiments, the microbe is genetically modified by transfection/transformation with a recombinant DNA plasmid encoding one or more of the LEKTI protein domains and one or more antibiotic resistance genes. For example, some embodiments of the recombinant DNA plasmid comprise a kanamycin resistance gene and/or a trimethoprim resistance gene; e.g. dfrA. According to some embodiments, treatment of the skin of the mammal with an antibiotic (for which the recombinant microbe is resistant) may be used to bias the population of commensal microbes toward a larger proportion of LEKTI producing microbes. Other elements that may be present in the recombinant DNA plasmid include, without limitation, a replication protein gene, such as a member of the Rep superfamily of replication proteins. For example, according to some embodiments the recombinant DNA plasmid comprises the repF gene.

According to some embodiments, the recombinant DNA plasmid comprises one or more sequences of the pJB38 vector. According to some embodiments, the recombinant LEKTI is operably linked to an inducible promoter, ribosome binding site, export signal, and/or cell penetrating peptide in the pJB38 vector. As used herein, the term “pJB38-LEKTI-complete” means a recombinant DNA plasmid construct comprising the pJB38 vector and one or more LEKTI domains.

According to some embodiments, the recombinant DNA plasmid comprises the pKK30-LEKTI as shown in FIG. 1. According to some embodiments, the present disclosure provides a composition for the treatment of a cancer, for delaying the progression of a cancer and/or preventing the recurrence of a cancer comprising a microbe comprising the pKK30-LEKTI-plasmid as shown in FIG. 1.

According to some such embodiments, the microbe is selected from the group consisting of Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp. According to some embodiments, bacteria in the microbial compositions comprise one or more of Staphylococcus epidermidis, Staphylococcus hominis, Staphylococcus warneri, Streptococcus pyogenes, Streptococcus mitis, Lactobacillus acidophilus, Propionibacterium acnes, Acinetobacter johnsonii, and Pseudomonas aeruginosa and mixtures thereof.

According to some embodiments of any one of the above aspects, the microbe is a Staphylococcus spp.

According to some embodiments, the amount or durations of availability of therapeutic LEKTI protein is controlled by the stability of the vector harboring the LEKTI in a microbe. For example, the persistence of a recombinant vector may be controlled by one or more elements of a plasmid including those that provide host-beneficial genes, plasmid stability mechanisms, and plasmid co-adaptation. For example, some plasmid may provide for stable replication, active partitioning mechanisms, and mechanisms that insure reliable inheritance of plasmids to daughter cells over generations. (See, e.g., J. C. Baxter, B. E. Funnell, Plasmid partition mechanisms, Microbiol. Spectr., 2 (2014) PLAS-0023-2014 and Nils Huller et al., An evolutionary perspective on plasmid lifestyle modes, Current Opinion in Microbiology, Volume 38, August 2017, Pages 74-80, each of which are incorporated by herein by reference in its entirety) According to some embodiments, the present invention includes the use of all conventional selection and stability methods known to a person of skill in the art.

### 3. Vector Encoded LEKTI Domains

LEKTI nucleic acids may be expressed from transcription units inserted into DNA or RNA vectors (see, e.g., Couture, A, et al., TIG. (1996), 12:5-10; WO 00/22113, WO 00/22114, and U.S. Pat. No. 6,054,299). According some embodiments, a LEKTI nucleic acid is comprised in a vector, such as a viral expression vector. According some embodiments, the LEKTI nucleic acid comprises SEQ ID NO: 119, or a fragment thereof. According to some embodiments, the LEKTI nucleic acid is at least 75%, at least 80%, at least 85%, at least 90%, at least 95%, at least 96%, at least 97%, at least 98% or at least 99% identical to SEQ ID NO: 119. According some embodiments, the LEKTI nucleic acid comprises SEQ ID NO: 119, or a fragment thereof. According to some embodiments, the LEKTI nucleic acid is at least 75%, at least 80%, at least 85%, at least 90%, at least 95%, at least 96%, at least 97%, at least 98% or at least 99% identical to SEQ ID NO: 128.

In some embodiment, expression is sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

Delivery of a LEKTI (e.g., LEKTI D6) expressing vector can be systemic, such as by intravenous or intramuscular administration, by administration to target cells ex-planted from the patient followed by reintroduction into the patient, or by any other means that allows for introduction into a desired target cell. According to some embodiments, delivery is intratumoral.

In certain embodiment, the nucleic acids described herein or the nucleic acids encoding a protein described herein, e.g., an effector, are incorporated into a vector, e.g., a viral vector.

The individual strand or strands of a LEKTI (e.g., LEKTI D6) nucleic acid molecule can be transcribed from a promoter in an expression vector. Where two separate strands are to be expressed to generate, for example, a dsRNA, two separate expression vectors can be co-introduced (e.g., by transfection or infection) into a target cell. Alternatively, each individual strand of a nucleic acid molecule can be transcribed by promoters both of which are located on the same expression plasmid. In one embodiment, a nucleic acid molecule is expressed as inverted repeat polynucleotides joined by a linker polynucleotide sequence such that the nucleic acid molecule has a stem and loop structure.

Expression vectors are generally DNA plasmids or viral vectors. Expression vectors compatible with eukaryotic cells, preferably those compatible with vertebrate cells, can be used to produce recombinant constructs for the expression of LEKTI (e.g., LEKTI D6) as described herein.

Constructs for the recombinant expression of LEKTI (e.g., LEKTI D6) will generally require regulatory elements, e.g., promoters, enhancers, etc., to ensure the expression of LEKTI (e.g., LEKTI D6) in target cells.

Expression of natural or synthetic nucleic acids is typically achieved by operably linking a nucleic acid encoding the nucleic acid of interest to a regulatory region, such as a promoter, and incorporating the construct into an expression vector. The vectors can be suitable for replication and integration in eukaryotes.

Regulatory regions, such as a promoter, suitable for operable linking to a nucleic acid molecules can be operably linked to a regulatory region such as a promoter. can be from any species. Any type of promoter can be operably linked to a nucleic acid sequence. Examples of promoters include, without limitation, tissue-specific promoters, constitutive promoters, and promoters responsive or unresponsive to a particular stimulus (e.g., inducible promoters). Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-la (EF-la). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the present invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Additional regulatory regions that may be useful in nucleic acid constructs, include, but are not limited to, transcription and translation terminators, initiation sequences, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Useful selectable markers include, for example, antibiotic-resistance genes, such as neo and the like. Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

Signal peptides may also be included and can be used such that an encoded polypeptide is directed to a particular cellular location (e.g., the cell surface).

Reporter genes may be used for identifying potentially transfected cells and for evaluating the functionality of transcriptional control sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Other aspects to consider for vectors and constructs are known in the art.

In some embodiments, a vector, e.g., a viral vector comprises a LEKTI (e.g., LEKTI D6) comprising a site-specific LEKTI (e.g., LEKTI D6) targeting moiety comprising a nucleic acid molecule.

Viral vector systems which can be utilized with the methods and compositions described herein include, but are not limited to, (a) adenovirus vectors (e.g., an Ad5/F35 vector); (b) retrovirus vectors, including but not limited to lentiviral vectors (including integration competent or integration-defective lentiviral vectors), moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. See, e.g., U.S. Pat. Nos. 6,534,261; 6,607,882; 6,824,978; 6,933,113; 6,979,539; 7,013,219; and 7,163,824, the entire contents of each of which is incorporated by reference herein.

Vectors, including those derived from retroviruses such as adenoviruses and adeno-associated viruses and lentiviruses, are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art, and described in a variety of virology and molecular biology manuals.

In one embodiment, a suitable viral vector for use in the present invention is an adeno-associated viral vector, such as a recombinant adeno-associate viral vector.

Recombinant adeno-associated virus vectors (rAAV) are gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). AAV serotypes, including AAV1, AAV2, AAV3, AAV4, AAV5, AAV6, AAV7, AAV8 and AAV9, can be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

### 4. Pharmaceutical Compositions

Aspects of the present disclosure include one or more of the LEKTI protein domains described herein, in combination with a pharmaceutically acceptable carrier. The compounds are preferably combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosures of which are hereby incorporated herein by reference, in their entirety.

A pharmaceutical composition is formulated to be compatible with its intended route of administration. Pharmaceutical compositions as described herein may be administered to a mammalian host in a variety of forms adapted to the chosen route of administration, e.g., orally or parenterally. Parenteral administration includes administration by the following routes: intramuscular; subcutaneous; intraocular; intrasynovial; transepithelial including transdermal, ophthalmic, sublingual and buccal; topically, including ophthalmic, dermal, ocular, and rectal; and nasal inhalation via insufflations and aerosols, including nasopharyngeal and throat installation.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

According to some embodiments, pharmaceutical compositions comprising the viral vectors described herein are administered intratumorally in vivo by injection (e.g., into a primary tumor or a secondary tumor (metastatic tumor)).

There are several ways to administer viral vectors with therapeutic genes (e.g., LEKTI, LEKTI D6) into solid tumors that grow in a mammalian animal body. For example, cancer gene therapy protocols use direct injection of the recombinant vector into the tumor (e.g., Haddada et al., Biochem. Biophys. Res. Comm. 195:1174-1183, 1993; Vincent et al., Hum. Gene Ther. 7:197-205, 1996, incorporated by reference in their entireties herein). All established tumors, both primary and metastatized, that are larger than a few millimeter in diameter are vascularized (Folkman et al., J. Nat. Cancer Inst. 82:4, 1990; Folkman and Shing, J. Biol. Chem. 267:10931-10934, 1992). Thus, an alternative way of delivering genetic material into solid tumors and/or their metastases could be by administering the recombinant viral vectors via the blood or lymphatic circulation.

The active compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

According to some embodiments the compositions comprising a microbe genetically modified to express one or more LEKTI protein domains encoded by one or more SPINK genes for use according to the present invention can comprise any pharmaceutically effective amount of the recombinant bacteria to produce a therapeutically effective amount of the desired polypeptide or therapeutically effective domain(s) thereof, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about. 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0% or more by weight of recombinant bacteria, the upper limit of which is about 90.0% by weight of recombinant, bacteria.

According to some embodiments, the composition for use according to the present invention can comprise, for example, at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to about 5%, or more by weight of recombinant bacteria.

According to some embodiments, the composition is a topical formulation. According to some embodiments, the topical formulation can be in any form suitable for application to the body surface, such as a cream, lotion, sprays, solution, gel, ointment, paste, plaster, paint, bioadhesive, suspensions, emulsions, or the like, and/or can be prepared so as to contain liposomes, microsomes, micelles, and/or microspheres. Such a formulation can be used in combination with an occlusive overlayer so that moisture evaporating from the body surface is maintained within the formulation upon application to the body surface and thereafter.

According to some embodiments, the formulation can include a living cell culture composition and can comprise at least one engineered bacterial strain that produces a therapeutically effective recombinant polypeptide or therapeutically effective domain(s) thereof. This engineered living cell culture composition can deliver the polypeptide directly to the skin for treating or preventing abnormal skin conditions.

Topical formulations include those in which any other active ingredient(s) is (are) dissolved or dispersed in a dermatological vehicle known in the art (e.g. aqueous or nonaqueous gels, ointments, water-in-oil or oil-in-water emulsions). Constituents of such vehicles can comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as MIGLYOL™, or silicone oils such as dimethicone). Depending, inter alia, upon the nature of the formulation as well as its intended use and site of application, the dermatological vehicle employed can contain one or more components (for example, when the formulation is an aqueous gel, components in addition to water) selected from the following list: a solubilizing agent or solvent (e.g. a β-cyclodextrin, such as bydroxypropyl β-cyclodextrin, or an alcohol or polyol such as ethanol, propylene glycol or glycerol); a thickening agent (e.g. hydroxyethylceliulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

According to some embodiments, the pharmaceutical composition of the invention can be applied in combination with (solid) carriers or matrices such as dressing(s), band aid(s) or tape(s). The compound(s) can be covalently or non-covalently bound to said carrier or matrix.

For example, the compound(s) may be incorporated into a dressing to be applied over a lesion. Examples of such dressings include staged or layered dressings incorporating slow-release hydrocolloid particles containing the composition or sponges containing the wound healing material optionally covered by conventional dressings. The concentration of a solution of the pharmaceutical composition to be immobilized in a matrix of a wound dressing is generally in the range of 0.001 to 1% (w/v) preferably 0.01-0.1% (w/v). Furthermore, the compound(s) as recited above can be incorporated into a suitable material capable of administering the enzyme to a wound in a slow release or controlled release manner.

According to some embodiments, a topical formulation may also include a skin lightening agent. Suitable skin lightening agents include, but are not limited to, ascorbic acid and derivatives thereof; kojic acid and derivatives thereof; hydroquinone; azelaic acid; and various plant extracts, such as those from licorice, grape seed, and bear berry. A skin conditioning agent includes, for example, a substance that enhances the appearance of dry or damaged skin, as well as a material that adheres to the skin to reduce flaking, restore suppleness, and generally improve the appearance of skin. Representative examples of a skin conditioning agent that may be used include: acetyl cysteine, N-acetyl dihydrosphingosine, acrylates/behenyl acrylate/dimethicone acrylate copolymer, adenosine, adenosine cyclic phosphate, adenosine phosphate, adenosine triphosphate, alanine, albumen, algae extract, allantoin and derivatives, aloe barbadensis extracts, amyloglucosidase, arbutin, arginine, bromelain, buttermilk powder, butylene glycol, calcium gluconate, carbocysteine, carnosine, beta-carotene, casein, catalase, cephalins, ceramides, Chamomilla recutita (matricaria) flower extract, cholecalciferol, cholesteryl esters, coco-betaine, corn starch modified, crystallins, cycloethoxymethicone, cysteine DNA, cytochrome C, darutoside, dextran sulfate, dimethicone copolyols, dimethylsilanol hyaluronate, elastin, elastin amino acids, ergocalciferol, ergosterol, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin. Other non-limiting examples of a skin conditioning agent that may be included in the compositions includes lactoferrin, lanosterol, lecithin, linoleic acid, linolenic acid, lipase, lysine, lysozyme, malt extract, maltodextrin, melanin, methionine, niacin, niacinamide, oat amino acids, oryzanol, palmitoyl hydrolyzed proteins, pancreatin, papain, polyethylene glycol, pepsin, phospholipids, phytosterols, placental enzymes, placental lipids, pyridoxal 5-phosphate, quercetin, resorcinol acetate, riboflavin, saccharomyces lysate extract, silk amino acids, sphingolipids, stearamidopropyl betaine, stearyl palmitate, tocopherol, tocopheryl acetate, tocopheryl linoleate, ubiquinone, Vitis vinifera (grape) seed oil, wheat amino acids, xanthan gum, and zinc gluconate. Skin protectant agents include, for example, a compound that protects injured or exposed skin or mucous membrane surfaces from harmful or irritating external compounds. Representative examples include algae extract, allantoin, aluminum hydroxide, aluminum sulfate, Camellia sinensis leaf extract, cerebrosides, dimethicone, glucuronolactone, glycerin, kaolin, lanolin, malt extract, mineral oil, petrolatum, potassium gluconate, and talc.

An emollient may be included in a pharmaceutical composition of the disclosure. An emollient generally refers to a cosmetic ingredient that can help skin maintain a soft, smooth, and pliable appearance. Emollients typically remain on the skin surface, or in the stratum corneum, to act as a lubricant and reduce flaking. Some examples of an emollient include acetyl arginine, acetylated lanolin, algae extract, apricot kernel oil polyethylene glycol-6 esters, avocado oil polyethylene glycol-11 esters, bis-polyethylene glycol-4 dimethicone, butoxyethyl stearate, glycol esters, alkyl lactates, caprylyl glycol, cetyl esters, cetyl laurate, coconut oil polyethylene glycol-10 esters, alkyl tartrates, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, odontella aurita oil, 2-oleamido-1,3 octadecanediol, palm glycerides, polyethylene glycol avocado glycerides, polyethylene glycol castor oil, polyethylene glycol-22/dodecyl glycol copolymer, polyethylene glycol shea butter glycerides, phytol, raffinose, stearyl citrate, sunflower seed oil glycerides, and tocopheryl glucoside.

Humectants are cosmetic ingredients that help maintain moisture levels in skin. Examples of humectants include acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxy polyethylene glycol, myristamidobutyl guanidine acetate, polyglyceryl sorbitol, potassium pyrollidone carboxylic acid (PCA), propylene glycol, sodium pyrollidone carboxylic acid (PCA), sorbitol, sucrose, and urea.

A pharmaceutically acceptable carrier can also be incorporated in the compositions of the present invention and can be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers. The term “pharmaceutically acceptable” or “pharmaceutically acceptable carrier” is used herein to refer to a compound or composition that can be incorporated into a pharmaceutical formulation without causing undesirable biological effects or unwanted, interaction with other components of the formulation, “Carriers” or “vehicles” as used herein refer to carrier materials suitable for incorporation in a topically applied composition. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue.

A film former, when it dries, forms a protective film over the site of application. The film inhibits removal of the active ingredient and keeps it in contact with the site being treated. An example of a film former that is suitable for use in this invention is Flexible Collodion, US P. As described in Remington: The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction, and are typically liquid or semiliquid preparations in which particles, including the active agent, are present in a water or alcohol base. Lotions are usually suspensions of solids, and preferably, comprise a liquid oily emulsion of the oil-in-water type. Lotions are preferred formulations herein for treating large body areas, because of the ease of applying a more fluid composition. It is generally necessary that the insoluble matter in a lotion be finely-divided.

Lotions will typically contain suspending agents to produce better dispersions as well as compounds useful for localizing and holding the active agent in contact with the skin, e.g., methylcellulose, sodium earhoxymethyl-celiulose, or the like.

Solutions are homogeneous mixtures prepared by dissolving one or more chemical substances (solutes) in a liquid such that the molecules of the dissolved substance are dispersed among those of the solvent. The solution can contain other pharmaceutically or cosmetically acceptable chemicals to buffer, stabilize or preserve the solute. Common examples of solvents used in preparing solutions are ethanol, water, propylene glycol or any other acceptable vehicles. As is of course well known, gels are semisolid, suspension-type systems. Single-phase gels contain organic macromolecules distributed substantially uniformly throughout the carrier liquid, which is typically aqueous, but also, preferably, contain an alcohol, and, optionally, an oil. Preferred “organic macromolecules,” i.e., gelling agents, are cross-linked acrylic acid polymers such as the “carbomer” family of polymers, e.g., carboxypolyalkylenes that can be obtained commercially under the Carbopol trademark. Also preferred are hydrophilic polymers such as polyethylene oxides, polyoxyethylene-polyoxypropylene copolymers and polyvinylalcohol; cellulosic polymers such as hydroxy-propyl cellulose, hydroxyethyl cellulose, hydroxypropyl methylcellulose, hydroxy-propyl methylcellulose phthaiate, and methylcellulose; gums such as tragacanth and xanthan gum; sodium alginate; and gelatin, In order to prepare a uniform gel, dispersing agents such as alcohol or glycerin can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, Pa.: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Emulsifiable ointment bases, also known as absorbent ointment bases, contain little or no water and include, for example, hydroxystearin sulfate, anhydrous lanolin, and hydrophilic petrolatum.

Emulsion ointment bases are either water-in-oil (W/O) emulsions or oil-in-water (O/W) emulsions, and include, for example, acetyl alcohol, glyceryl monostearate, lanolin, and stearic acid. Preferred water-soluble ointment bases are prepared from polyethylene glycols of varying molecular weight; see Remington: The Science and Practice of Pharmacy for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Enhancers are those lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter δ of plasticizing enhancers is in the range of about 2.5 to about 10, preferably in the range of about 5 to about 10. Preferred lipophilic enhancers are fatty esters, fatty alcohols, and fatty ethers. Examples of specific and most preferred fatty acid esters include methyl laurate, ethyl oleate, propylene glycol nionolaurace, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995)(incorporated herein by reference).

Various other additives can be included in the compositions of the present invention in addition to those identified above. These include, but are not limited to, antioxidants, astringents, perfumes, preservatives, emollients, pigments, dyes, humectants, propeliants, and sunscreen agents, as well as other classes of materials whose presence can be pharmaceutically or otherwise desirable. Typical examples of optional additives for inclusion in the formulations of the invention are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (vitamin C), a-tocopherol (Vitamin E), β-tocopherol, γ-tocopherol, δ-tocopherol, ε-tocopherol, ξι-tocopherol, Z∧-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the Carbopol trademark. Other additives include beneficial agents such as those materials that condition the skin (particularly, the upper layers of the skin in the Stratum corneum) and keep it soft by retarding the decrease of its water content and/or protect the skin. Such conditioners and moisturizing agents include, by way of example, pyrrolidine carboxylic acid and amino acids; anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants such as ascorbic acid (Vitamin C), a-tocopherol (Vitamin E), β-tocopherol, γ-tocopherol, 6-tocopherol, ε-tocopherol, ξι-tocopherol, ξ₂-tocopherol, η-tocopherol, and retinol (Vitamin A), and/or pharmaceutically acceptable salts, esters, amides, or other derivatives thereof. A preferred tocopherol compound is a-tocopherol. Additional agents include those that are capable of improving oxygen supply in skin tissue, as described, for example, in Gross, et al, WO 94/00098 and Gross, et al, WO 94/00109, both assigned to Lancaster Group AG (incorporated herein by reference). Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycmnamate, octyl salicylate, oxybenzone, padirnate O, phenylbenzirmdazole sulfonic acid, sulisobenzone, titanium dioxide, trolamine salicylate, zinc oxide, ensulizole, meradiraate, octinoxate, octisalate, and octocrylene. See Title 21. Chapter 1. Subchapter D. Part 352. “Sunscreen drug products for over-the-counter human use” incorporated herein in its entirety.

Other embodiments can include a variety of non-carcinogenic, non-irritating healing materials that facilitate treatment with the formulations of the invention. Such healing materials can include nutrients, minerals, vitamins, electrolytes, enzymes, herbs, plant extracts, glandular or animal extracts, or safe therapeutic agents that can be added to the formulation to facilitate the healing of dermal disorders.

The amounts of these various additives are those conventionally used in the cosmetics field, and range, for example, from about 0.01% to about 20% of the total weight of the topical formulation.

The compositions of the invention can also include conventional additives such as opacifiers, fragrance, colorant, stabilizers, surfactants, and the like. In certain embodiments, other agents can also be added, such as preservative agents, to prevent spoilage upon storage, i.e., to inhibit growth of microbes such as yeasts and molds.

The compositions can also contain irritation-mitigating additives to minimize or eliminate the possibility of skin irritation or skin damage resulting from the chemical entity to be administered, or other components of the composition.

Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphophilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than about 20 wt. %, more typically not more than about 5 wt. %, of the formulation.

Further suitable pharmacologically active agents that can be incorporated into the present formulations in certain embodiments and thus topically applied along with the active agent include, but are not limited to, the following: agents that improve or eradicate pigmented or non-pigmented age spots, keratoses, and wrinkles; antipruritic and antixerotic agents; anti-inflammatory agents; local anesthetics and analgesics; corticosteroids; retinoids; vitamins; hormones; and antimetabolites.

Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycmnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The application regimen will depend on a number of factors that can readily be determined, such as the severity of the condition and its responsiveness to initial treatment, but will normally involve one or more applications per day on an ongoing basis. One of ordinary skill can readily determine the optimum amount of the formulation to be administered, administration methodologies and repetition rates. In general, it is contemplated that the formulations of the invention will be applied in the range of once or twice weekly up to once or twice daily.

The pharmaceutical compositions of the invention comprise one or more active ingredients, e.g. therapeutic agents, in admixture with one or more pharmaceutically-acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21st Edition, Lippincott Williams and Wilkins, Philadelphia, Pa.).

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The pharmaceutical compositions of the present invention suitable for parenteral administrations may comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

According to some embodiments, the pharmaceutical compositions described herein can be used in combination with one or more blocking antibodies targeted to an immune “checkpoint” molecule such as for example, BTLA, HVEM, TIM3, GAL9, LAG3, VISTA, KIR, 2B4, CD160 (also referred to as BY55), CGEN-15049, CHK 1 and CHK2 kinases, A2aR, CEACAM (e.g., CEACAM-1, CEACAM-3 and/or CEACAM-5), GITR, GITRL, galectin-9, CD244, CD160, TIGIT, SIRPα, ICOS, CD172a, and TMIGD2 and various B-7 family ligands (including, but are not limited to, B7-1, B7-2, B7-DC, B7-H1, B7-H2, B7-H3, B7-H4, B7-H5, B7-H6 and B7-H7).

### 5. Methods of Treatment

According to some embodiments, the present disclosure is based, in part, on administering one or more LEKTI protein domains to inhibit pathways activated by protease targets of LEKTI, including pathways involved in cellular proliferation, morphology, adhesion, invasion and expression of key MMPs involved in tumor progression. As such, the methods of the present disclosure can be used in treating, preventing or delaying the recurrence of cancer by inhibiting pathways activated by protease targets of LEKTI. The methods of the present disclosure can also be used in treating, preventing or delaying the recurrence of tumor cell invasion and metastasis, by inhibiting pathways activated by protease targets of LEKTI.

Provided herein are methods of treating a subject afflicted with cancer, comprising administering one or more LEKTI protein domains to the subject in need thereof. Provided herein are methods of preventing the recurrence of a cancer in a subject afflicted with the cancer, wherein the cancer is in remission, comprising administering one or more LEKTI protein domains to the subject in need thereof. Provided herein are methods of preventing the progression of a cancer in a subject afflicted with the cancer, comprising administering one or more LEKTI protein domains to the subject in need thereof. Provided herein are methods of preventing a cancer in a subject with risk factors for developing the cancer, comprising administering one or more LEKTI protein domains to the subject in need thereof.

According to some embodiments, the one or more LEKTI protein domain are one or more of domains D1, D2, D3, D4, D5, D6, D7, D8, D9, D10, D11, D12, D13, D14, and D15.

According to some embodiments, the LEKTI domain comprises LEKTI domain 6 (D6).

According to some embodiments, the one or more LEKTI protein domains are encoded by a nucleic acid. According to some embodiments, the nucleic acid comprises a sequence that is at least 85% identical, at least 90% identical, at least 95% identical, at least 96% identical, at least 97% identical, at least 98% identical, or at least 99% identical to SEQ ID NO: 119, or fragments thereof. According to some embodiments, the nucleic acid is comprised in a vector. According to some embodiments, the vector is a viral expression vector. According to some embodiments, the vector is comprised within a cell.

Also provided herein are methods of treating a subject afflicted with cancer, comprising administering a microbe genetically modified to express one or more LEKTI protein domains encoded by one or more SPINK genes to the subject in need thereof. Provided herein are methods of preventing the recurrence of a cancer in a subject afflicted with the cancer, wherein the cancer is in remission, comprising administering a microbe genetically modified to express one or more LEKTI protein domains encoded by one or more SPINK genes to the subject in need thereof. Provided herein are methods of preventing the progression of a cancer in a subject afflicted with the cancer, comprising administering a microbe genetically modified to express one or more LEKTI protein domains encoded by one or more SPINK genes to the subject in need thereof. Provided herein are methods of preventing a cancer in a subject with risk factors for developing the cancer, comprising administering a microbe genetically modified to express one or more LEKTI protein domains encoded by one or more SPINK genes to the subject in need thereof.

Risk factors include, but are not limited to, genetic mutation, exposure to carcinogens, exposure to radiation, certain viruses, unprotected exposure to the sun, smoking and lifestyle choices (e.g., diet and exercise).

Cancers and related disorders that can be prevented, treated, or managed by methods and compositions of the present invention include but are not limited to the following cancers: lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma, Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyo sarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochrmocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but not limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to squamous cell carcinoma, and adenocarcinoma; uterine cancers such as but not limited to endometrial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcionomas such as but not limited to pappilary, nodular, and diffuse; lung cancers such as non-small cell lung cancer, squamous cell carcinoma (epidermoid carcinoma), adenocarcinoma, large-cell carcinoma, and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; oral cancers such as but not limited to squamous cell carcinoma; basal cancers; salivary gland cancers such as but not limited to adenocarcinoma, mucoepidermoid carcinoma, and adenocystic carcinoma; pharynx cancers such as but not limited to squamous cell cancer, and verrucous; skin cancers such as but not limited to basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxsarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America) Accordingly, the compositions and methods of the invention are useful in the treatment or prevention of a variety of cancers or other abnormal proliferative diseases, including (but not limited to) the following: carcinoma, including that of the bladder, breast, prostate, rectal, colon, kidney, liver, lung, ovary, pancreas, stomach, cervix, thyroid and skin; including squamous cell carcinoma; hematopoictic tumors of myeloid lineage, including acute and chronic myelogenous leukemias and promoclocytic leukemia; tumors of mesenchymal origin; including fibrosarcoma and rhabdomyoscarcoma; other tumors, including melanoma, seminoma, tetratocarcinoma, neuroblastoma, and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, hormone dependent tumors of the breast, prostate and ovary, and precancerous lesions such as familial adenomatous polyposis, and myelodysplastic syndromes. In specific embodiments, malignancy or dysproliferative changes (such as metaplasis and dyplasias), or hyperproliferative disorders, are treated in the skin, lung, colon, rectum, breast, prostate, bladder, kidney, pancreas, ovary, or uterus. According to some embodiments, sarcoma, melanoma, small lung carcinoma, or leukemia is treated.

According to some embodiments, the cancer is malignant. According to some embodiments, the disorder to be treated is a pre-cancerous condition. According to some embodiments, the pre-cancerous condition is actinic keratosis, high-grade prostatic intraepithelial neoplasia (PIN), fibroadenoma of the breast, or fibrocystic disease.

According to some embodiments, the cancer is selected from the group consisting of malignant melanoma, colon cancer, breast cancer, lung cancer, ovarian cancer, gastric cancer, oral tongue squamous cell carcinoma, squamous cell cancer, prostate cancer, pancreatic cancer, liver cancer, kidney cancer, bladder cancer, cervical cancer, endometrial cancer, gallbladder cancer, brain cancer and oral cancer.

According to some embodiment, the cancer is a companion cancer in an subject that is not a human subject, for example a mammal that is not a human, such as domesticated animals (e.g., cows, sheep, cats, dogs, and horses), non-human primates (e.g., monkeys), rabbits, and rodents (e.g., mice and rats).

According to some embodiments, the therapy of the present invention (e.g., administering one or more LEKTI protein domains) effectively increases the duration of survival of the subject. In some embodiments, administering one or more LEKTI protein domains of the present invention increases the progression-free survival of the subject. In certain embodiments, administering one or more LEKTI protein domains of the present invention increases the progression-free survival of the subject in comparison to standard-of-care therapies.

After the administration of one or more LEKTI protein domains, the subject having a cancer tumor can exhibit an overall survival of at least about 10 months, at least about 11 months, at least about 12 months, at least about 13 months, at least about 14 months at least about 15 months, at least about 16 months, at least about 17 months, at least about 18 months, at least about 19 months, at least about 20 months, at least about 21 months, at least about 22 months, at least about 23 months, at least about 2 years, at least about 3 years, at least about 4 years, or at least about 5 years or more after the administration. According to some embodiments, the duration of survival or the overall survival of the subject is increased by at least about 1 month, at least about 2 months, at least about 3 months, at least about 4 months, at least about 6 months, or at least about 1 year when compared to another subject treated with only a standard-of-care therapy. For example, the duration of survival or the overall survival of the subject treated with one or more LEKTI protein domains as described herein is increased by at least about 5%, at least about 10%, at least about 15%, at least about 20%, at least about 25%, at least about 30%, at least about 40%, at least about 50% or at least about 75% when compared to another subject treated with only a standard-of-care therapy.

Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers.

According to some embodiments, the therapy of the present invention effectively increases the duration of progression free survival of the subject. In some embodiments, the subject exhibits a progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years.

According to some embodiments, the subject includes those who responded to prior treatment and may be characterized with stable disease, those who have failed to respond or to respond adequately to prior treatment, those who may have responded to prior treatment but then experienced progression of the disease, and those who may have had such response followed by progression more than once.

According to some embodiments, the described invention utilizes a sample obtained from a subject, wherein the subject has been diagnosed with a cancer. According to some embodiments, the described invention utilizes a sample obtained from a subject, wherein the subject is at risk of developing a cancer (as determined, e.g., by genetic testing, family history or risk factors). The sample can include, but is not limited to, a tissue sample, a blood sample, a serum sample, a urine sample, a saliva sample and the like. According to some embodiments, the sample is a tumor sample. Methods of obtaining a sample from a subject are well known in the art. Such methods include, but are not limited to, biopsy, such as for example, a core biopsy or a fine needle biopsy. The sample may be a fresh, a frozen or a fixed, wax-embedded sample. Non-limiting examples of fixed, wax-embedded samples include formalin-fixed, paraffin-embedded samples. According to some embodiments, the subject sample comprises a SPINK5 gene mutation. According to some embodiments, the subject sample has been found to have elevated levels of KLKS protein compared to a suitable control sample. According to some embodiments, the subject sample has been found to have elevated levels of PRP2 protein compared to a suitable control sample. According to some embodiments, the subject sample has been found to have elevated levels of TSLP protein compared to a suitable control sample. According to some embodiments, the subject sample has been found to have elevated levels of a cathelicidin protein (e.g. LL-32) compared to a suitable control sample. According to some embodiments, the subject sample has been found to have elevated levels of an MMP protein compared to a suitable control sample.

In certain embodiments, the subject is a chemotherapy-naive patient (e.g., a patient who has not previously received any chemotherapy). In other embodiments, the subject for the present combination therapy has received another cancer therapy (e.g., a chemotherapy), but is resistant or refractory to such another cancer therapy. In certain embodiments, the subject has cancer cells that are squamous. In certain embodiments, the subject has cancer cells that are non-squamous.

Cutaneous Neoplasms

The principal cutaneous neoplasms are actinic or solar keratoses (pre-malignant skin lesions), basal cell carcinomas, squamous cell carcinomas, condyloma acuminatum, cutaneous t-cell lymphoma (i.e. mycosis fungoides), and malignant melanomas. Of these lesions, solar keratoses, basal cell carcinomas, and condyloma acumination are by far the most common. These are also the easiest to treat and generally have a good prognosis. Squamous cell carcinomas of the skin, cutaneous T-cell lymphomas, and malignant melanomas are less common, but carry a much poorer prognosis.

According to some embodiments, the pre-cancerous condition is actinic keratosis (AK). AK is a pre-cancerous form of hyperkeratosis that is known to be caused by frequent or extreme exposure of the skin to sunlight. AK typically presents as small, rough patches of skin approximately 2 mm to 7 mm in diameter. The patches are usually reddish in color, with rough texture and whitish or yellow scales. Actinic keratosis is frequently painful, and may develop to a malignant condition without treatment and upon continued exposure to the sun. According to some embodiments, the disclosure provides methods for treating actinic keratosis, comprising identifying an area of a person's skin that exhibits lesions indicating actinic keratosis; applying to that area a composition comprising a LEKTI protein, or a portion thereof; monitoring the skin area regularly to determine whether the skin lesions remain; and repeating said applying step at least twice daily for a period of at least two months until the area is substantially free of the lesions.

Melanoma is an aggressive malignancy associated with five-year survival rates under 5% in patients with metastatic disease. Patients with lymph node involvement >1 mm, including those detected only by sentinel lymph node biopsy, are at high risk of both local and distant relapse after definitive surgery due to the frequent presence of distant micrometastatic disease at presentation (Van Akkooi et al. 2009 J Clin. Oncol. vol. 27, 2009, pages 15s). Approximately half of these patients will ultimately die of metastatic disease (Markovic, 2007. Mayo Clin. Proc vol. 82, 2007, pages 490-513), and the morbidity from uncontrolled relapses is also considerable.

Basal cell carcinomas usually present initially as a small, dome-shaped bump. The bump may be covered by small, superficial blood vessels that cause the spot to appear shiny and translucent, sometimes looking “pearly.” Some basal cell carcinomas contain melanin pigment, making them look dark rather than shiny. According to some embodiments, the disclosure provides methods for treating basal cell carcinoma, comprising identifying an area of a person's skin that exhibits lesions indicating basal cell carcinoma; applying to that area a treating composition comprising a LEKTI protein, or a portion thereof; monitoring the skin area regularly to determine whether the skin lesions remain; and repeating said applying step several times daily for a period of at least two months until the area is substantially free of the lesions.

Provided herein are methods of treating a subject afflicted with a cutaneous neoplasm, comprising administering one or more LEKTI protein domains to the subject in need thereof. Provided herein are methods of preventing the recurrence of a cutaneous neoplasm in a subject afflicted with melanoma, wherein the cutaneous neoplasm is in remission, comprising administering one or more LEKTI protein domains to the subject in need thereof. Provided herein are methods of preventing the progression of a cutaneous neoplasm in a subject afflicted with the cutaneous neoplasm, comprising administering one or more LEKTI protein domains to the subject in need thereof. Provided herein are methods of preventing a cutaneous neoplasm in a subject with risk factors for developing the cutaneous neoplasm, comprising administering one or more LEKTI protein domains to the subject in need thereof.

Provided herein are methods of treating a subject afflicted with a cutaneous neoplasm, comprising administering a microbe genetically modified to express one or more LEKTI protein domains encoded by one or more SPINK genes to the subject in need thereof. Provided herein are methods of preventing the recurrence of a cutaneous neoplasm in a subject afflicted with melanoma, wherein the cutaneous neoplasm is in remission, comprising administering a microbe genetically modified to express one or more LEKTI protein domains encoded by one or more SPINK genes to the subject in need thereof. Provided herein are methods of preventing the progression of a cutaneous neoplasm in a subject afflicted with the cutaneous neoplasm, comprising administering a microbe genetically modified to express one or more LEKTI protein domains encoded by one or more SPINK genes to the subject in need thereof. Provided herein are methods of preventing a cutaneous neoplasm in a subject with risk factors for developing the cutaneous neoplasm, comprising administering a microbe genetically modified to express one or more LEKTI protein domains encoded by one or more SPINK genes to the subject in need thereof.

According to some embodiments, the cutaneous neoplasm is a melanoma. According to some embodiments, the melanoma is malignant melanoma. According to some embodiments, the malignant melanoma is Stage I/II. According to some embodiments, the malignant melanoma is stage III. According to some embodiments, the malignant melanoma is Stage IV. According to some embodiments, the malignant melanoma is on the face, neck, head arms, legs, feet, chest or back. According to some embodiments, the malignant melanoma is in the mouth.

According to some embodiments, the cutaneous neoplasm is a squamous cell carcinoma. According to some embodiments, the squamous cell carcinoma is on the face, neck, head arms, legs, feet, chest or back. According to some embodiments, the squamous cell carcinoma is an oral cancer, including the tongue, oropharynx and oral cavity, lips and gums.

Colorectal Cancer

Colorectal cancer is a cancer that starts in the colon or the rectum. These cancers can also be named colon cancer or rectal cancer, depending on where they start. Excluding skin cancers, colorectal cancer is the third most common cancer diagnosed in both men and women in the United States. The American Cancer Society's estimates for the number of colorectal cancer cases in the United States for 2019 are 101,420 new cases of colon cancer and 44,180 new cases of rectal cancer. Overall, the lifetime risk of developing colorectal cancer i: about 1 in 22 (4.49%) for men and 1 in 24 (4.15%) for women. In the United States, colorectal cancer is the third leading cause of cancer-related deaths in men and in women, and the second most common cause of cancer deaths when men and women are combined. The American Cancer Society expects that colorectal cancer is expected to cause about 51,020 deaths during 2019.

The stage (extent of spread) of a colorectal cancer depends on how deeply it grows into the wall and if it has spread outside the colon or rectum. The staging system most often used for colorectal cancer is the American Joint Committee on Cancer (AJCC) TNM system, which is based on the extent (size) of the tumor (T), the spread to nearby lymph nodes (N), the spread (metastasis) to distant sites (M) (AJCC Cancer Staging Manual, Eighth Edition (2017), published by Springer International Publishing). The stages can be summarized as follows:

Stage 0: called cancer in situ. The cancer cells are only in the mucosa, or the inner lining, of the colon or rectum. According to some embodiments, the colorectal cancer is Stage 0.

Stage I: the cancer has grown through the mucosa and has invaded the muscular layer of the colon or rectum. It has not spread into nearby tissue or lymph nodes (T1 or T2, N0, M0). According to some embodiments, the colorectal cancer is Stage I.

Stage IIA: the cancer has grown through the wall of the colon or rectum and has not spread to nearby tissue or to the nearby lymph nodes (T3, N0, M0). According to some embodiments, the colorectal cancer is Stage IIA.

Stage JIB: The cancer has grown through the layers of the muscle to the lining of the abdomen, called the visceral peritoneum. It has not spread to the nearby lymph nodes or elsewhere (T4a, N0, M0). According to some embodiments, the colorectal cancer is Stage IIB.

Stage IIC: the tumor has spread through the wall of the colon or rectum and has grown into nearby structures. It has not spread to the nearby lymph nodes or elsewhere (T4b, N0, M0). According to some embodiments, the colorectal cancer is Stage IIC.

Stage IIIA: the cancer has grown through the inner lining or into the muscle layers of the intestine. It has spread to 1 to 3 lymph nodes or to a nodule of tumor in tissues around the colon or rectum that do not appear to be lymph nodes but has not spread to other parts of the body (T1 or T2, N1 or N1c, M0; or T1, N2a, M0). According to some embodiments, the colorectal cancer is Stage IIIA.

Stage IIIB: the cancer has grown through the bowel wall or to surrounding organs and into 1 to 3 lymph nodes or to a nodule of tumor in tissues around the colon or rectum that do not appear to be lymph nodes. It has not spread to other parts of the body (T3 or T4a, N1 or N1c, M0; T2 or T3, N2a, M0; or T1 or T2, N2b, M0). According to some embodiments, the colorectal cancer is Stage IIIB.

Stage IIIC: the cancer of the colon, regardless of how deep it has grown, has spread to 4 or more lymph nodes but not to other distant parts of the body (T4a, N2a, M0; T3 or T4a, N2b, M0; or T4b, N1 or N2, M0). According to some embodiments, the colorectal cancer is Stage IIIC.

Stage IVA: the cancer has spread to a single distant part of the body, such as the liver or lungs (any T, any N, M1a). According to some embodiments, the colorectal cancer is Stage IVA.

Stage IVB: the cancer has spread to more than 1 part of the body (any T, any N, M1b). According to some embodiments, the colorectal cancer is Stage IVB.

Stage IVC: the cancer has spread to the peritoneum. It may also have spread to other sites or organs (any T, any N, M1c). According to some embodiments, the colorectal cancer is Stage IVC.

Provided herein are methods of treating a subject afflicted with colon cancer, comprising administering one or more LEKTI protein domains to the subject in need thereof. Provided herein are methods of preventing the recurrence of colon cancer in a subject afflicted with colon cancer, wherein the colon cancer is in remission, comprising administering one or more LEKTI protein domains to the subject in need thereof. Provided herein are methods of preventing the progression of colon cancer in a subject afflicted with colon cancer, comprising administering one or more LEKTI protein domains to the subject in need thereof. Provided herein are methods of preventing colon cancer in a subject with risk factors for developing colon cancer, comprising administering one or more LEKTI protein domains to the subject in need thereof.

Provided herein are methods of treating a subject afflicted with colon cancer, comprising administering a microbe genetically modified to express one or more LEKTI protein domains encoded by one or more SPINK genes to the subject in need thereof. Provided herein are methods of preventing the recurrence of colon cancer in a subject afflicted with colon cancer, wherein the colon cancer is in remission, comprising administering a microbe genetically modified to express one or more LEKTI protein domains encoded by one or more SPINK genes to the subject in need thereof. Provided herein are methods of preventing the progression of colon cancer in a subject afflicted with colon cancer, comprising administering a microbe genetically modified to express one or more LEKTI protein domains encoded by one or more SPINK genes to the subject in need thereof. Provided herein are methods of preventing colon cancer in a subject with risk factors for developing colon cancer, comprising administering a microbe genetically modified to express one or more LEKTI protein domains encoded by one or more SPINK genes to the subject in need thereof.

Lung Cancer

Provided herein are methods of treating a subject afflicted with lung cancer, comprising administering one or more LEKTI protein domains to the subject in need thereof. Provided herein are methods of preventing the recurrence of lung cancer in a subject afflicted with lung cancer, wherein the lung cancer is in remission, comprising administering one or more LEKTI protein domains to the subject in need thereof. Provided herein are methods of preventing the progression of lung cancer in a subject afflicted with lung cancer, comprising administering one or more LEKTI protein domains to the subject in need thereof. Provided herein are methods of preventing lung cancer in a subject with risk factors for developing lung cancer, comprising administering one or more LEKTI protein domains to the subject in need thereof.

Provided herein are methods of treating a subject afflicted with lung cancer, comprising administering a microbe genetically modified to express one or more LEKTI protein domains encoded by one or more SPINK genes to the subject in need thereof. Provided herein are methods of preventing the recurrence of lung cancer in a subject afflicted with lung cancer, wherein the lung cancer is in remission, comprising administering a microbe genetically modified to express one or more LEKTI protein domains encoded by one or more SPINK genes to the subject in need thereof. Provided herein are methods of preventing the progression of lung cancer in a subject afflicted with lung cancer, comprising administering a microbe genetically modified to express one or more LEKTI protein domains encoded by one or more SPINK genes to the subject in need thereof. Provided herein are methods of preventing lung cancer in a subject with risk factors for developing lung cancer, comprising administering a microbe genetically modified to express one or more LEKTI protein domains encoded by one or more SPINK genes to the subject in need thereof.

As non-small cell lung carcinoma (NSCLC) comprises more than 85% of lung tumors, in embodiments the lung cancer is NSCLC. The majority of patients (approximately 78%) are diagnosed with advanced/recurrent or metastatic disease. Metastases to the adrenal gland from lung cancer are a common occurrence, with about 33% of patients having such metastases. here is a particular unmet need among patients who have squamous cell NSCLC (representing up to 25% of all NSCLC) as there are few treatment options after first line (1L) therapy.

The present methods can treat a non-squamous NSCLC of any stages. There are at least seven stages used for NSCLC: occult (hidden) stage, Stage 0 (carcinoma in situ), Stage I, Stage II, Stage IIIA, Stage IIIB, and Stage IV. In the occult stage, the cancer cannot be seen by imaging or bronchoscopy. In Stage 0, cancer cells are found in the lining of the airways.

According to some embodiments, the subject never smoked. According to some embodiments, the subject formerly smoked. According to some embodiments, the subject currently smokes.

According to some embodiments, the present methods treat a Stage I non-squamous NSCLC. Stage I NSCLC is divided in Stage IA and IB. In Stage IA, the tumor is in the lung only and is 3 centimeters or smaller. In Stage IB, the cancer has not spread to the lymph nodes and one or more of the following is true: 1) the tumor is larger than 3 centimeters but not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus.

According to some embodiments, the methods of the present invention treat a Stage II non-squamous NSCLC. Stage II NSCLC is divided into Stage IIA and IIB. In Stage IIA, the cancer has either spread to the lymph nodes or not. If the cancer has spread to the lymph nodes, then the cancer can only have spread to the lymph nodes on the same side of the chest as the tumor, the lymph nodes with cancer or within the lung or near the bronchus, and one or more of the following is true: 1) the tumor is not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIA if the cancer has not spread to the lymph nodes and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. In stage IIB, the cancer has either spread to the lymph nodes or not. If the cancer has spread to the lymph nodes, then the cancer can only have spread to the lymph nodes on the same side of the chest as the tumor, the lymph nodes with cancer are within the lung or near the bronchus and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIB if the cancer has not spread to the lymph nodes and one or more of the following is true: 1) the tumor is larger than 7 centimeters; 2) the cancer has spread to the main bronchus (and is at least 2 centimeters below where the trachea joins the bronchus), the chest wall, the diaphragm, or the nerve that controls the diaphragm; 3) cancer has spread to the membrane around the heart or lining the chest wall; 4) the whole lung has collapsed or developed pneumonitis (inflammation of the lung); or 5) there are one or more separate tumors in the same lobe of the lung.

According to some embodiments, any methods of the present invention treat Stage III non-squamous NSCLC. Stage IIIA is divided into 3 sections. These 3 sections are based on 1) the size of the tumor; 2) where the tumor is found and 3) which (if any) lymph nodes have cancer. In the first type of Stage IIIA NSCLC, the cancer has spread to the lymph nodes on the same side of the chest as the tumor, and the lymph nodes with the cancer are near the sternum or where the bronchus enters the lung. Additionally: 1) the tumor may be any size; 2) part of the lung (where the trachea joins the bronchus) or the whole lung may have collapsed or developed pneumonitis (inflammation of the lung); 3) there may be one or more separate tumors in the same lobe of the lung; and 4) cancer can have spread to any of the following: a) main bronchus, but not the area where the trachea joins the bronchus, b) chest well, c) diaphragm and the nerve that controls it, d) membrane around the lung or lining the chest wall, e) membrane around the heart. In the second type of Stage IIIA NSCLC, the cancer has spread to the lymph nodes on the same side of the chest as the tumor, and the lymph nodes with the cancer are within the lung or near the bronchus. Additionally: 1) the tumor may be any size; 2) the whole lung may have collapsed or developed pneumonitis (inflammation of the lung); 3) there may be one or more separate tumors in the any of the lobes of the lung with cancer; and 4) cancer can have spread to any of the following: a) main bronchus, but not the area where the trachea joins the bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the third type of Stage IIIA NSCLC, the cancer has not spread to the lymph nodes, the tumor may be any size, and cancer has spread to any one of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi). Stage IIIB is divided into 2 sections depending on 1) the size of the tumor, 2) where the tumor is found, and 3) which lymph nodes have cancer. In the first type of Stage IIIB NSCLC, the cancer has spread to the lymph nodes on the opposite side of the chest as the tumor. Additionally, 1) the tumor may be any size; 2) part of the lung (where the trachea joins the bronchus) or the whole lung may have collapsed or developed pneumonitis (inflammation of the lung); 3) there may be one or more separate tumors in any of the lobs of the lung with cancer; and 4) cancer may have spread to any of the following: a) main bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the second type of Stage MB NSCLC, the cancer has spread to lymph nodes on the same side of the chest as the tumor. The lymph nodes with cancer are near the sternum (chest bone) or where the bronchus enters the lung. Additionally, 1) the tumor may be any size; 2) there may be separate tumors in different lobes of the same lung; and 3) cancer has spread to any of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi).

In some embodiments, the methods of the invention treat a Stage IV non-squamous NSCLC. In Stage IV NSCLC, the tumor may be any size and the cancer may have spread to the lymph nodes. One or more of the following is true in Stage IV NSCLC: 1) there are one or more tumors in both lungs; 2) cancer is found in the fluid around the lungs or heart; and 3) cancer has spread to other parts of the body, such as the brain, liver, adrenal glands, kidneys or bone.

Gastric Cancer

Gastric (or stomach) cancer tends to develop slowly over many years. There is a wide variation in prognosis of gastric tumors. Tumors in the distal stomach are more often cured than those in the gastric cardiac or gastroesophageal junction. The depth to which the tumor invades the stomach wall and whether lymph nodes are involved influence the likelihood of cure. The World Health Organization has histopathologically classified stomach cancer into ten major groups: (1) adenocarcinoma, (2) papillary adenocarcinoma, (3) tubular adenocarcinoma, (4) mucinous adenocarcinoma, (5) signet ring cell carcinoma, (6) adenosquamous carcinoma, (7) carcinoid tumor, (8) mixed carcinoid-adenocarcinoma), (9) small cell carcinoma, and undifferentiated carcinoma. Adenocarcinoma is by far the most common stomach cancer followed by malignant lymphoma.

The staging of stomach cancer is based on the revised criteria of TNM staging by the American Joint Committee for Cancer (AJCC) published in 1988 (Stages 0 to IV).

Provided herein are methods of treating a subject afflicted with gastric cancer, comprising administering one or more LEKTI protein domains to the subject in need thereof. Provided herein are methods of preventing the recurrence of gastric cancer in a subject afflicted with gastric cancer, wherein the gastric cancer is in remission, comprising administering one or more LEKTI protein domains to the subject in need thereof. Provided herein are methods of preventing the progression of gastric cancer in a subject afflicted with gastric cancer, comprising administering one or more LEKTI protein domains to the subject in need thereof. Provided herein are methods of preventing gastric cancer in a subject with risk factors for developing gastric cancer, comprising administering one or more LEKTI protein domains to the subject in need thereof.

Provided herein are methods of treating a subject afflicted with gastric cancer, comprising administering a microbe genetically modified to express one or more LEKTI protein domains encoded by one or more SPINK genes to the subject in need thereof. Provided herein are methods of preventing the recurrence of gastric cancer in a subject afflicted with gastric cancer, wherein the gastric cancer is in remission, comprising administering a microbe genetically modified to express one or more LEKTI protein domains encoded by one or more SPINK genes to the subject in need thereof. Provided herein are methods of preventing the progression of gastric cancer in a subject afflicted with gastric cancer, comprising administering a microbe genetically modified to express one or more LEKTI protein domains encoded by one or more SPINK genes to the subject in need thereof. Provided herein are methods of preventing gastric cancer in a subject with risk factors for developing gastric cancer, comprising administering a microbe genetically modified to express one or more LEKTI protein domains encoded by one or more SPINK genes to the subject in need thereof.

Ovarian Cancer

Provided herein are methods of treating a subject afflicted with ovarian cancer, comprising administering one or more LEKTI protein domains to the subject in need thereof. Provided herein are methods of preventing the recurrence of ovarian cancer in a subject afflicted with ovarian cancer, wherein the ovarian cancer is in remission, comprising administering one or more LEKTI protein domains to the subject in need thereof. Provided herein are methods of preventing the progression of ovarian cancer in a subject afflicted with ovarian cancer, comprising administering one or more LEKTI protein domains to the subject in need thereof. Provided herein are methods of preventing ovarian cancer in a subject with risk factors for developing ovarian cancer, comprising administering one or more LEKTI protein domains to the subject in need thereof.

Provided herein are methods of treating a subject afflicted with ovarian cancer, comprising administering a microbe genetically modified to express one or more LEKTI protein domains encoded by one or more SPINK genes to the subject in need thereof. Provided herein are methods of preventing the recurrence of ovarian cancer in a subject afflicted with ovarian cancer, wherein the ovarian cancer is in remission, comprising administering a microbe genetically modified to express one or more LEKTI protein domains encoded by one or more SPINK genes to the subject in need thereof. Provided herein are methods of preventing the progression of ovarian cancer in a subject afflicted with ovarian cancer, comprising administering a microbe genetically modified to express one or more LEKTI protein domains encoded by one or more SPINK genes to the subject in need thereof. Provided herein are methods of preventing ovarian cancer in a subject with risk factors for developing ovarian cancer, comprising administering a microbe genetically modified to express one or more LEKTI protein domains encoded by one or more SPINK genes to the subject in need thereof.

Ovarian cancer ranks as the fifth most common cancer in women and has the highest mortality rate among gynecologic malignancies (Suh et al. Expert Rev Mol Diagn 2010; 10: 1069-83; Landen et al. J Clin Oncol 2008; 26:995-1005). Although the 5-year survival rate of ovarian cancer is around 90% when detected in early stages (I/II), nearly 80% of the new cases are diagnosed in advanced stages (III/IV) because of the asymptomatic nature of the disease at stage I and early stage II. The 5-year survival rate of advanced ovarian cancer is as low as 11% (Altekruse et al. SEER Cancer Statistics Review, 1975-2007, National Cancer Institute. Bethesda, Md.).

Ovarian cancer is not a single disease but consists of more than 30 types and subtypes of malignancies, each with its own histopathologic appearance and biologic behavior. Generally, ovarian cancers are grouped into 3 major categories: (1) epithelial tumors (tumors arising from cells that line or cover the ovaries); (2) germ cell tumors (tumors that originate from cells that are destined to form eggs within the ovaries; and (3) sex cord-stromal cell tumors (tumors that begin in connective cells that hold the ovaries together and produce female hormones). The most common ovarian cancers are epithelial tumors, which account for about 90% of all ovarian cancers. Ovarian epithelial tumors are divided into subtypes which include serous, papillary serous, endometrioid, mucinous and clear cell tumors.

Ovarian Cancer Staging

The process used to determine whether ovarian cancer has spread within the ovaries or to other parts of the body (i.e., metastasized) is called staging. It is important to determine the stage of ovarian cancer because the stage will determine the type of treatment plan selected to combat the disease. The results of tests used to diagnose ovarian cancer are often also used to stage the disease. Such tests include ultrasound, computerized tomography (CT) scan, positron emission tomography (PET) scan, magnetic resonance imaging (MRI), X-ray and biopsy. Ovarian cancer staging guidelines have been developed by the International Federation of Gynecologists and Obstetricians (FIGO). The FIGO staging system for ovarian cancer is shown in Table 4.

TABLE 4
FIGO Ovarian Cancer Staging

| Stage | Area of Involvement |
|---|---|
| IA | Tumor limited to 1 ovary, capsule intact, no tumor on surface, |
| | negative washings |
| IB | Tumor involves both ovaries; otherwise like IA |
| IC1 | Surgical spill |
| IC2 | Capsule rupture before surgery or tumor on ovarian surface |
| IC3 | Malignant cells in the ascites or peritoneal washings |
| IIA | Extension and/or implant on uterus and/or Fallopian tubes |
| IIB | Extension to other pelvic intraperitoneal tissues |
| IIIA1 | Positive retroperitoneal lymph nodes only |
| | IIIA1(i) - Metastasis ≤ 10 mm |
| | IIIA1(ii) - Metastasis > 10 mm |
| IIIA2 | Microscopic, extrapelvic (above the brim) peritoneal |
| | involvement ± positive retroperitoneal lymph nodes |
| IIIB | Macroscopic, extrapelvic, peritoneal metastasis ≤ 2 cm ± |
| | positive retroperitoneal lymph nodes; includes extension |
| | to capsule of liver/spleen |

IIIC Macroscopic, extrapelvic, peritoneal metastasis >2 cm+positive retroperitoneal lymph nodes; includes extension to capsule of liver/spleen

IVA Pleural effusion with positive cytology

IVB Hepatic and/or splenic parenchymal metastasis, metastasis to extra-abdominal organs

(including inguinal lymph nodes and lymph nodes outside of the abdominal cavity)

Ovarian Cancer Grading

In addition to staging, an ovarian tumor can also be described by grade (G). Grading determines how similar ovarian cancer tissue is to normal tissue. Tumor grade is determined by microscopic examination of cancer tissue; with healthy cells appearing as well-differentiated. That is, the more differentiated the ovarian tumor, the better the prognosis. The ovarian cancer grading system is shown in Table 5.

TABLE 5
Ovarian Cancer Grading

| Grade | Description |
|---|---|
| GX | Grade cannot be evaluated |
| GB | Tissue considered borderline cancerous; low |
| | malignant potential |
| G1 | Tissue is well-differentiated (healthy cells) |
| G2 | Tissue is moderately differentiated (more abnormal |
| | than healthy cells) |
| G3 to G4 | Tissue is poorly differentiated or undifferentiated |

Early Stage (FIGO Stage I-II) Ovarian Cancer

Due to the lack of effective screening programs, ovarian cancer is diagnosed at an early stage only in about 25% of cases (Kim et al., Journal of Experimental & Clinical Cancer Research 2012, 31: 14). In most of these cases, surgery is able to cure the disease, and the five-year survival rate for early-stage (stage I or II) ovarian cancer is around 90% (Hennessy et al., Lancet 2009, 374: 1371-82). Adjuvant chemotherapy for early stage ovarian cancer is still controversial, but some studies have shown its benefit under confined conditions. According to these studies, patients with IA or IB FIGO stage, non-clear-cell histology, well-differentiated (G1) tumors, and an “optimal” surgery (i.e., performed according to international guidelines, with pelvic and retroperitoneal assessment), appear not to benefit from chemotherapy (Trimbos J B et al., J Natl Cancer Inst 2003, 95: 105-112). Thus, it is commonly believed that, at least in these cases, chemotherapy can probably be avoided and patients can be advised to undergo clinical and instrumental follow-up. In all the other (early stage) patients, (adjuvant) chemotherapy is indicated (Hennessy B T, et al., Lancet 2009, 374: 1371-82).

Advanced (FIGO Stage III-IV) Ovarian Cancer

The standard treatment for patients with advanced ovarian cancer is maximal surgical cytoreduction (i.e., total abdominal hysterectomy, bilateral salpingo-oophorectomy, pelvic and para-aortic lymphadenectomy and omentectomy) followed by systemic platinum-based chemotherapy (e.g., cisplatin followed by carboplatin-based combinations, cisplatin with paclitaxel, cisplatin with cyclophosphamide, cisplatin with doxorubicin, etc.). The expected 5-year survival for these patients is 10-30% (Hennessey B T et al., Lancet 2009, 374: 1371-82).

In a 2015 study, Dorn et al. showed that ovarian cancer patients release significant amounts of KLKS into the blood stream, while in patients with benign ovarian tumor, KLKS is also released into the blood of these patients but at a much lower rate. (Dorn et al. Journal of Clinical Oncology 28, no. 15_suppl (May 20, 2010) 5088-5088). Further, Dorn et al. found that KLKS content both in ascitic fluid and serum was of prognostic relevance regarding progression-free survival of the ovarian cancer patients.

According to some embodiments, the disclosure provides compositions and methods for treating early stage ovarian cancer in a subject. According to some embodiments, the disclosure provides compositions and methods for preventing early stage ovarian cancer in a subject. According to some embodiments, the disclosure provides compositions and methods for delaying the progression of early stage ovarian cancer in a subject. According to some embodiments, the disclosure provides compositions and methods for treating late stage ovarian cancer in a subject. According to some embodiments, the disclosure provides compositions and methods for preventing late stage ovarian cancer in a subject. According to some embodiments, the disclosure provides compositions and methods for delaying the progression of late stage ovarian cancer in a subject. According to some embodiments, the disclosure provides methods of preventing the progression from early stage ovarian cancer to late stage ovarian cancer.

Oral Tongue Squamous Cell Carcinoma (OTSCC)

Oral squamous cell carcinoma affects about 34,000 people in the US each year. In the US, 3% of cancers in men and 2% in women are oral squamous cell carcinomas, most of which occur after age 50. As with most head and neck sites, squamous cell carcinoma is the most common oral cancer. The chief risk factors for oral squamous cell carcinoma are smoking (especially >2 packs/day) and alcohol use. The combination of heavy smoking and alcohol abuse is estimated to raise the risk 100-fold in women and 38-fold in men. Squamous cell carcinoma of the tongue may also result from any chronic irritation, such as dental caries, overuse of mouthwash, chewing tobacco, or the use of betel quid. About 40% of intraoral squamous cell carcinomas begin on the floor of the mouth or on the lateral and ventral surfaces of the tongue.

Provided herein are methods of treating a subject afflicted with OTSCC, comprising administering one or more LEKTI protein domains to the subject in need thereof. Provided herein are methods of preventing the recurrence of OTSCC in a subject afflicted with OTSCC, wherein the OTSCC is in remission, comprising administering one or more LEKTI protein domains to the subject in need thereof. Provided herein are methods of preventing the progression of OTSCC in a subject afflicted with OTSCC, comprising administering one or more LEKTI protein domains to the subject in need thereof. Provided herein are methods of preventing OTSCC in a subject with risk factors for developing OTSCC, comprising administering one or more LEKTI protein domains to the subject in need thereof.

Provided herein are methods of treating a subject afflicted with OTSCC, comprising administering a microbe genetically modified to express one or more LEKTI protein domains encoded by one or more SPINK genes to the subject in need thereof. Provided herein are methods of preventing the recurrence of OTSCC in a subject afflicted with OTSCC, wherein the OTSCC is in remission, comprising administering a microbe genetically modified to express one or more LEKTI protein domains encoded by one or more SPINK genes to the subject in need thereof. Provided herein are methods of preventing the progression of OTSCC in a subject afflicted with OTSCC, comprising administering a microbe genetically modified to express one or more LEKTI protein domains encoded by one or more SPINK genes to the subject in need thereof. Provided herein are methods of preventing OTSCC in a subject with risk factors for developing OTSCC, comprising administering a microbe genetically modified to express one or more LEKTI protein domains encoded by one or more SPINK genes to the subject in need thereof.

Esophageal Cancer

According to Global cancer statistics 2018, esophageal cancer is the ninth most common tumor in the world. In 2018, there were 572 034 new cases of esophageal cancer. In addition, there were 508 585 (5.3%) patients with esophageal cancer died, which is the sixth leading cause of cancer death worldwide (Bray et al. (2018) CA Cancer J Clin. 2018; 68:394-424). Despite improvement of esophageal cancer diagnosis and treatment measures, the prognosis of patients with esophageal cancer is still poor, and the 5-year survival rate ranges from only 10% to 25% (Ohashi et al. (2015) Gastroenterology. 2015; 149:1700-1715). Wang et al. (Cancer Medicine (2019) Volume 8, Issue 5: 2360-2371) detected the expression level of SPINK5 protein in 12 cases of esophageal cancer tissues and their matched normal esophageal tissues by immunohistochemistry, and showed that the expression of SPINK5 protein was significantly reduced in 11 cases (11/12) of esophageal cancer, compared with normal esophageal tissues. Further, Wang et al. found that the expression level of SPINK5 protein in 205 cases of esophageal cancer was closely related to lymph node metastasis and pathological differentiation.

Provided herein are methods of treating a subject afflicted with esophageal cancer, comprising administering one or more LEKTI protein domains to the subject in need thereof. Provided herein are methods of preventing the recurrence of esophageal cancer in a subject afflicted with esophageal cancer, wherein the esophageal cancer is in remission, comprising administering one or more LEKTI protein domains to the subject in need thereof. Provided herein are methods of preventing the progression of esophageal cancer in a subject afflicted with esophageal cancer, comprising administering one or more LEKTI protein domains to the subject in need thereof. Provided herein are methods of preventing esophageal cancer in a subject with risk factors for developing esophageal cancer, comprising administering one or more LEKTI protein domains to the subject in need thereof.

Provided herein are methods of treating a subject afflicted with esophageal cancer, comprising administering a microbe genetically modified to express one or more LEKTI protein domains encoded by one or more SPINK genes to the subject in need thereof. Provided herein are methods of preventing the recurrence of esophageal cancer in a subject afflicted with esophageal cancer, wherein the esophageal cancer is in remission, comprising administering a microbe genetically modified to express one or more LEKTI protein domains encoded by one or more SPINK genes to the subject in need thereof. Provided herein are methods of preventing the progression of esophageal cancer in a subject afflicted with esophageal cancer, comprising administering a microbe genetically modified to express one or more LEKTI protein domains encoded by one or more SPINK genes to the subject in need thereof. Provided herein are methods of preventing esophageal cancer in a subject with risk factors for developing esophageal cancer, comprising administering a microbe genetically modified to express one or more LEKTI protein domains encoded by one or more SPINK genes to the subject in need thereof.

According to some embodiments, the method is part of a therapeutic regimen combining one or more additional agents as part of a therapeutic regimen for treating, preventing or delaying the recurrence of cancer. When used in this way, administration of a microbe genetically modified to express one or more LEKTI protein domains encoded by one or more SPINK genes can act additively or synergistically with other agents.

According to some embodiments, an additional agent may be selected from antineoplastic agents, radioiodinated compounds, toxins, other cytostatic or cytolytic drugs, and so forth. Antineoplastic therapeutics are well known and include: aminoglutethimide, azathioprine, bleomycin sulfate, busulfan, carmustine, chlorambucil, cisplatin, cyclophosphamide, cyclosporine, cytarabidine, dacarbazine, dactinomycin, daunorubicin, doxorubicin, taxol, etoposide, fluorouracil, interferon-α, lomustine, mercaptopurine, methotrexate, mitotane, procarbazine HCl, thioguanine, vinblastine sulfate and vincristine sulfate. According to some embodiments, the additional agent is a checkpoint inhibitor (see, e.g., WO 2013/173223, incorporated by reference in its entirety herein). According to some embodiments, the checkpoint inhibitor is an antibody therapeutic. According to some embodiments, the checkpoint inhibitor is an anti-PD-L1, anti-PD-1 or anti-CTLA4 antibody.

The present disclosure provides dosage regimens that can provide a desired response, e.g., a maximal therapeutic response and/or minimal adverse effects.

According to some embodiments, the methods described herein comprise multiple treatments (e.g., 1, 2, 3, 4, 5, 6, 7, 8, 9, 10, 15, 20, 25, 20, 35, 40, 45, 50, 55, 60, 65, 70, 75, 80, 85, 90, 95, 100 or more treatments).

Actual dosage levels of the active ingredients in the pharmaceutical compositions of the present invention can be varied so as to obtain an amount of the active ingredient that is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

### 6. Animal Models

The use of a microbe genetically modified to express one or more LEKTI protein domains encoded by one or more SPINK genes (in the presence or absence of additional therapeutic modalities) for treating, preventing or delaying the recurrence of cancer can be tested in one or more animal models. Exemplary animal models are described briefly herein. However, numerous animal models exist and any model available in the art can be readily used to evaluate a particular treatment regimen (e.g., to evaluate number of treatments, duration of treatment, combination with one or more current treatment modalities).

Nude Mouse Orthotopic Transplantation Tumor Models

The tumorigenic ability of cancer cells can be determined by orthotopic transplanted tumor model in nude mice. A nude mouse model of an orthotopic transplanted tumor can be established by subcutaneous injection of a tumorigenic cell line in 4-6 weeks old Balb/c nude mice. Animals are treated with a microbe genetically modified to express one or more LEKTI protein domains encoded by one or more SPINK genes (in the presence or absence of additional therapeutic modalities). After the nude mice are sacrificed, the tumor weight and tumor volume are observed and recorded.

Xenograft Transplantation Models

Patient-derived xenograft (PDX) mouse models are established by direct engraftment of patient-derived tumor fragments into immunocompromised mice. There have been several experimental protocols reported to generate PDX models, as individual research groups have their own ways to improve the success rate of PDX engraftment, although the protocols seem to share the fundamental concepts and techniques. Briefly, pieces of solid tumors or single-cell suspensions are collected from tumor tissues obtained by surgery or biopsy, and are transplanted under the skin (subcutaneous transplantation), in the same organ as the original tumors in the patients (orthotopic transplantation), or in the renal capsule in the recipient immunocompromised mouse. Subcutaneous transplantation models allow for easier cell transfer and precise monitoring of tumor formation and growth (Kim et al. Nat Protoc. 2009; 4(11):1670-80). In contrast, orthotopic PDX models are more difficult than heterotopic subcutaneous models for transplantation techniques and monitoring of tumor growth, but the microenvironments of transplanted tumors might be more similar to those of the original tumors in the patients. For example, it was reported that orthotopic PDX models showed increased incidence of metastases from transplanted pancreatic tumors, compared with heterotopic subcutaneous models (Fu et al. Proc Natl Acad Sci USA. 1992 Jun. 15; 89(12):5645-9).

Additionally, therapeutic regimens including a microbe genetically modified to express one or more LEKTI protein domains encoded by one or more SPINK genes (alone or in combination with one or more additional treatment modalities) can be tested in in vitro models (e.g., cell-based models, organ culture models). Further, such therapeutic regimens can be tested in vivo in human patients

### 7. Kits

According to another aspect, the present disclosure provides a kit for treating, preventing or delaying the progression of cancer in a subject in need thereof comprising: (1) a composition comprising a microbe genetically modified to express one or more LEKTI protein domains encoded by one or more SPINK genes; and (2) instructions for use.

The components of the kits may be packaged either in aqueous media or in lyophilized form. The kits will generally be packaged to include at least one vial, test tube, flask, bottle, syringe or other container means, into which the described reagents may be placed, and preferably, suitably aliquoted. Where additional components are provided, the kit will also generally contain a second, third or other additional container into which such component may be placed.

The container holds a composition which is by itself or combined with another composition effective for treating, preventing and/or diagnosing the condition and may have a sterile access por.

The following examples are provided to further illustrate the methods of the present invention. These examples are illustrative only and are not intended to limit the scope of the invention in any way.

### Example 1

### Bacteria

According to some embodiments, bacteria of the Staphylococcus aureus RN4220 strain may be used in preparation of the vector (Kreiswirth, B N et al. 1983). According to some such embodiments, a stock solution of the strain is stored at −20° C. in 50% glycerol in LB or TS broth.

According to some embodiments, bacteria of the Staphylococcus epidermidis strain ATCC 12228 or NRRL B-4268 may be used (Zhang, YQ., et ah 2003). According to some such embodiments, a stock solution of the strain is stored at −20° C. in 50% glycerol in LB broth or TS broth. Bacteria are cultured in LB broth or TS broth. After 16 hours of incubation, bacteria are harvested by centrifugation and 10-fold concentrated in LB broth or TS broth at 2×10⁹ bacteria/100 ul. A stock preparation of the bacteria is prepared by inoculating 5 mL broth with S. epidermidis and grown overnight at 30° C. Then, 3 mL fully grown culture is added to 1 ml 60% glycerol and stored at −80° C.

Expression Vector

According to some embodiments, plasmid construct pKK30-LEKTI-complete may comprise the pKK30 vector with a LEKTI domain insert. According to some embodiments, the LEKTI domain may be operably linked to a SecA secretion signal, a 6×His tag (SEQ ID NO: 120), and/or an RMR cell permeation sequence, with expression under the control of a chloramphenicol-resistance (CmR) promoter sequence (from pDB114E). According to some embodiments, the pKK30 vector comprises a dihydrofolate reductase (dfrA) selection gene.

Transformation

According to some embodiments, a vector harboring the LEKTI sequence may be transformed into the S. epidermidis strain. The vector harboring the LEKTI sequence may be prepared/transformed comprising the steps of: preparation of competent S. aureus bacterial cells, transformation of S. aureus, isolation of plasmid DNA from S. aureus, preparation of competent S. epidermidis bacterial cells, transformation of S. epidermidis, growth of transformed S. epidermidis bacteria, and storage of transformed S. epidermidis.

According to some embodiments, alternative intermediate strains can also be used for transformation and isolation of plasmid DNA in preparation for transformation into S. epidermidis. These strains may include but are not limited to E. coli strains among other bacteria, including those deficient in methylation.

According to some embodiments, S. aureus RN4220 cells may be made electrocompetent by growing 50 ml culture overnight in LB or TS medium at 37° C., then inoculating 100 ml fresh LB or TS medium with 10 ml of overnight culture. When OD₆₀₀ reaches 0.2-0.3, cells are pelleted and resuspended with 1× volume of 4° C. 10% sucrose. This process is repeated 3×, and then the cells are resuspended with 0.1× volume of 4° C. 10% sucrose, pelleted, and resuspended with 1 ml of 10% sucrose.

For transformation of RN4220, 200-500 ug of LEKTI plasmid (e.g. pKK30-LEKTI-complete) may be mixed with electrocompetent cells and transformed using electroporation at room temperature at 2.5 kV using the MicroPulser Electroporator (Bio-Rad, Hercules, Calif.). Transformed cells are plated at 28° C. overnight on selective LB or TB medium, grown overnight in selective LB or TB medium and then used to isolate DNA.

According to some embodiments, electrocompetent S. epidermidis ATCC 12228 or NRRL B-4268 are made using the following methods. First, 50 ml overnight culture of ATCC 12228 or NRRL B-4268 from a −80° C. stock are grown at 37° C. in B2 medium (1.0% tryptone, 2.5% yeast extract, 0.5% glucose, 2.5% NaCl, 0.1% K₂PO₄, pH to 7.5). 10 ml of overnight culture is diluted into fresh pre-warmed B2 media and shaken until OD₆₀₀ reaches 0.5-0.6 and then pelleted for 10 min at 4° C. Next, cells are washed with 1, 1/2, 1/20, and 1/50 volumes of cold 10% glycerol, pelleting at 4° C. between washes. The final pellet is resuspended in 700 ul of cold 10% glycerol.

According to some embodiments, electrocompetent ATCC 12228 or NRRL B-4268 are transformed with pKK30-LEKTI-complete, isolated from S. aureus, using electroporation at 2.5 kV, 25 uF, 100Ω. (normal reading is 4.5-5 msec using the Micropulser Electroporator (Bio-Rad, Hercules, Calif.)). Cells are then plated at 28° C. on selective LB or TB medium. According to some embodiments, transformation of the bacteria can also be performed via alternative methods of transformation including but not limited to alternative intermediate strains, bacteriophage transduction, and heat shock.

Analysis of Protein Expression

According to some embodiments, transformed cells are fractionated and analyzed via SDS-PAGE electrophoresis and western blotting. Bacterial cells expressing recombinant LEKTI and bacterial control cells are pelleted and lysed with CelLytic B Cell Lysis Reagent (Sigma-Aldrich, St. Louis, Mo.). The supernatant from the induced sample is collected and concentrated. Samples are resuspended in a reduced sample buffer and then electrophoresed on a 4-15% Tris-acrylimide gel with Tris-HCL running buffer. Following electrophoresis, the gel is transferred to a PVDF membrane, and sequentially probed with a primary goat monoclonal antibody against LEKTI domains 8-11 or a His tag. A horseradish peroxidase-conjugated donkey anti-goat antibody (sc-2020) is then probed and the secondary antibodies detected through autoradiography (Syngene GeneGnome Bio Imaging System) using enhanced chemiluminescence substrate (SuperSignal West Pico, Thermo Scientific).

Analysis of the supernatant and cell lysate demonstrates the successful expression and secretion of the therapeutic polypeptide upon transformation with a plasmid containing the protein of interest. Detection of protein expression and secretion is also possible using alternative methods and the current example should not be construed as a limitation to the present invention.

Treatment of Human Subjects

According to some embodiments, 1×10⁹ colony forming units (CFU) of S. epidermidis containing recombinant LEKTI can be added to a pharmaceutically acceptable carrier. The foregoing composition is useful for treating or preventing inflammatory skin diseases or disorders in a subject in need thereof. The composition can be applied at least once per day, up to for example about 3 to 4 times per day, or as needed or prescribed. According to some embodiments, only a single application is required to achieve a therapeutic effect. The composition can be used for as long as needed to ensure treatment of the condition or to continue to prevent the condition. The duration of treatment can vary from about 1 day up to about 10 to 14 days or longer. In certain instances, long term or chronic treatment can be administered.

### Example 2

Testing Serine Protease Inhibition Activity of Recombinant LEKTI

According to some embodiments, the protease inhibition activity of recombinant LEKTI is tested for differences achieved when operably linked to various secretion peptides and cell penetration peptides. According to some embodiments, specific combinations of secretion peptides and cell penetration peptides may have unpredictable effects on the protease inhibition function of the LEKTI domains, and therefore may be determined empirically.

According to some embodiments, LEKTI domains D8-D11, operably linked to a secretory tag, 6×His tag (SEQ ID NO: 120), and/or cell penetration tag, are cloned into an insect expression vector for large scale production of purified recombinant protein and assessed for inhibitory activity on one or more proteases (e.g. plasmin, cathepsin G, elastase, and trypsin).

Insect Cells and Reagents

The following reagents may be obtained commercially as indicated: Fall Army worm cell line Spodoptera frugiperda (Sf9), low-melting point agarose, cellFECTIN, pFASTBAC1, pCRII-TOPO, Escherichia colicompetent DH10BAC, cabbage looper egg cell line Trichoplusia ni 5B1-4 (High Five), and ultimate serum-free insect medium from Invitrogen (Carlsbad, Calif.); restriction endonucleases from New England Biolabs (Beverly, Mass.); TALON Superflow from Clontech Laboratory (Palo Alto, Calif.); Insect-XPRESS medium and fetal bovine serum from BioWhittaker (Walkersville, Md.); YM10 Centriplus from Millipore Corp. (Bedford, Mass.); precast SDS-PAGE gels, protein assay kit, SEC-250 size column, and prestained markers from Bio-Rad (Hercules, Calif.); BSA from Kabi Pharmacia (Franklin, Ohio); DTT and glycerol from Boehringer Mannheim Biochemicals (Indianapolis, Ind.); and penta-His mAb and six-His (SEQ ID NO: 120)tagged protein ladder from QIAGEN Inc. (Valencia, Calif.).

Cloning and Expression of LEKTI D8-D11

6×His (SEQ ID NO: 120) tagged LEKTI domains (e.g. SEQ ID NO: 109) operably linked to various permutations of secretion peptides and cell penetration peptides may be cloned into the pFASTBAC1 vector according to the manufacturers' instructions. Recombinant LEKTI composite viruses are then generated as previously described by Gao, M. et al., (1996) J. Biol. Chem. 271, 27782-27787, which is incorporated herein by reference in its entirety. To test the recombinant LEKTI composite viruses for recombinant LEKTI expression, Sf9 cells may be infected at varying multiplicities of infection with recombinant viruses, and the cell lysate and medium collected every 24-96 h. The presence of histidine-tagged protein may be confirmed by Western blot analysis using penta-His mAb directed against the six-histidine tag (SEQ ID NO: 120) as per the manufacturer's recommendations. LEKTI composite viruses that displayed the highest level of expression may be chosen for further experiments and spinner flasks.

The recombinant LEKTI protein may be produced on a large scale by infecting spinner cultures of Sf9 cells (1.6 billion cells) in 10% serum containing Insect-XPRESS medium at a multiplicity of infection of 8 plaque forming units (PFU). Three days after infection, the cell pellet may be harvested and the recombinant LEKTI selectively purified from the cell lysate using a Co²⁺-charged Sepharose affinity column (TALON) followed by SEC-250 size column chromatography, as previously described in Jayakumar, A. et al., (1995) Proc. Natl. Acad. Sci. U.S.A. 92, 8695-8699. Fractions containing homogeneous LEKTI may be pooled and concentrated by ultrafiltration. Protein may be quantified using the Bio-Rad Protein Assay Kit II.

Protease Inhibition Assay Reagents and Protocol

The following enzymes, chromogenic substrates, and reagents may be obtained commercially as indicated: human plasmin, human cathepsin L, human cathepsin S, human trypsin, human cathepsin G, human chymotrypsin, and human neutrophil elastase (HNE) from Athens Research & Technology, Inc. (Athens, Ga.); subtilisin A from Calbiochem-Novabiochem (San Diego, Calif.); papain from Roche Molecular Biochemicals (Indianapolis, Ind.); furin from New England BioLabs; succinyl-Ala-Ala-Pro-Phe-p-nitroanilide (Succ-AAPF-pNA) (SEQ ID NO: 121), succinyl-Ala-Ala-Val-pNA (Succ-AAVpNA), and D-Val-Leu-Lys-pNA (VLK-pNA) from Sigma Chemical Co. (St. Louis, Mo.); H-Glu-Gly-Arg-pNA (EGRpNA) and benzyloxycarbonyl-Phe-Arg-pNA (Z-FR-pNA) from Bachem Bioscience, Inc. (King of Prussia, Pa.); and methoxy-Succ-Arg-Pro-Tyr-pNA (MeO-Succ-RPY-pNA) from Chromogenix Instrumentation Laboratory SpA (Milan, Italy). PBS reaction buffer (137 mM NaCl, 27 mM KCl, and 10 mM phosphate buffer (pH 7.4)) may be used with trypsin, plasmin, cathepsin G, HNE, and chymotrypsin. Cathepsin reaction buffer (0.1% CHAPS, 50 mM sodium acetate (pH 5.5), 1 mM EDTA) may be used with cathepsins K, L, and S and papain. A unique reaction buffer may be used with subtilisin A (PBS and 0.1% Tween 20).

Proteinase inhibitory activity may be detected by the ability of recombinant LEKTI to block the cleavage of small, chromogenic peptide substrates as determined by a spectroscopy technique described previously in Schick, C. et al., (1998) Biochemistry 37, 5258-5266, which is incorporated herein by reference in its entirety. Inhibition of proteinase may be assessed after preincubating the enzyme with recombinant LEKTI for 2 min at 25° C. in 100 uL of assay buffer. This mixture may be added to 890 or 880 uL of assay buffer in a 1 mL quartz cuvette. The proteinase activity may be initiated by adding 10-20 uL of the appropriate pNA substrate. The change in absorbance at 405 nm (A₄₀₅=8.8 10⁻³ cm⁻¹) may be followed for as long as 10 min using a spectrophotometer (Beckman Instruments, Inc., Fullerton, Calif.). The rate changes (ΔA₄₀₅/min) of inhibited and control reactions may be determined from velocity plots.

According to some embodiments, different combinations of secretory tag and cell penetration tag may cause differing LEKTI protease activity on each of the tested proteases (e.g. trypsin, plasmin, cathepsin G, HNE, subtilisin A, and chymotrypsin). Furthermore, discrete combinations of secretory tag and cell penetration tag may cause differing LEKTI protease activity among individual proteases.

### Example 3

Penetrating Peptide Mediated Delivery

According to some embodiments, various combinations of secretory tag and cell penetration tag may affect the ability of the recombinant LEKTI protein to pass through a cell membrane to a greater or lesser degree. Thus, the various recombinant LEKTI products may be tested in cell culture to assess the effect of the various combinations of secretory tag and cell penetration tag.

According to some embodiments, adherent fibroblastic HS-68, NIH-3T3, 293, Jurkat T, or Cos-7 cell lines may be cultured in Dulbecco's modified Eagle's medium (DMEM) supplemented with 1% (vol/vol) 200 mM glutamine, 1% (vol/vol) antibiotics (streptomycin, 10,000 μg/ml; penicillin, 10,000 IU/ml), and 10% (wt/vol) FBS, at 37° C. in a humidified atmosphere containing 5% CO2. For peptide-mediated delivery of recombinant LEKTI proteins, purified recombinant LEKTI product (as obtained above) may be loaded in DMEM or PBS (500 μl of DMEM containing 0.25 μg of protein) and incubated for 30 min at 37° C. Cells grown to 75% confluency are then overlaid with these recombinant LEKTI protein media. After 30 min incubation at 37° C., 1 ml of fresh DMEM supplemented with 10% FBS is added to the cells, without removing the overlay of recombinant LEKTI protein, and cells are returned to the incubator for another 30 min. Cells are then extensively washed with PBS and examined for recombinant LEKTI protein. Cells could be observed by immunofluorescence by first fixing with 2% formalin (Sigma), permeabilizing, then incubating with primary anti-6×His tag (SEQ ID NO: 120) antibody and secondary antibody according to the manufacturers' instruction (“6×His” disclosed as SEQ ID NO: 120). Alternatively cells lysates could be obtained and the presence of His tagged recombinant LEKTI observed via Western blot, as described above.

According to some embodiments, certain combinations of secretory protein and penetrating peptide have differing effects on the ability of the recombinant LEKTI protein's ability to pass through the cell membrane.

### Example 4. Effect of LEKTI D6 on Head and Neck Squamous Cell Carcinoma (HNSCC) Cell Migration and Invasion

A well characterized tongue cancer cell line OSC-19 was used to study the effect of LEKTI D6 on HNSCC cancer cell migration and invasion.

Migration studies were undertaken using a BD BIOCOAT Tumor Invasion System that consists of a 24-Mutliwell-insert plate in which a PET membrane (8 μm pore size) has been coated without or with Matrigel.

The MATRIGEL® Invasion Chamber is useful to study cell invasion of cancer cells. Specific applications include assessment of the metastatic potential of tumor cells, inhibition of metastasis by extracellular matrix components or antineoplastic drugs (taxol), altered expression of cell surface proteins or metalloproteinases in metastatic cells, and cell invasion.

Purified recombinant LEKTI D6 protein was added exogenously to the upper wells of inserts, which were then were placed into lower wells containing NIH 3T3 supernatant containing 10% fetal calf serum as a chemo attractant. Cells were allowed to invade for 24 h in a CO₂ humidified incubator.

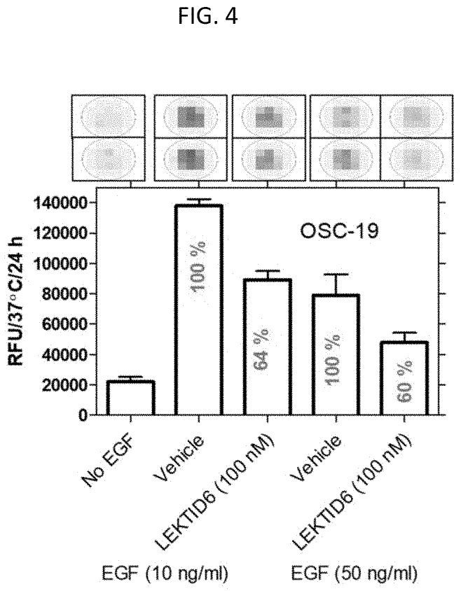

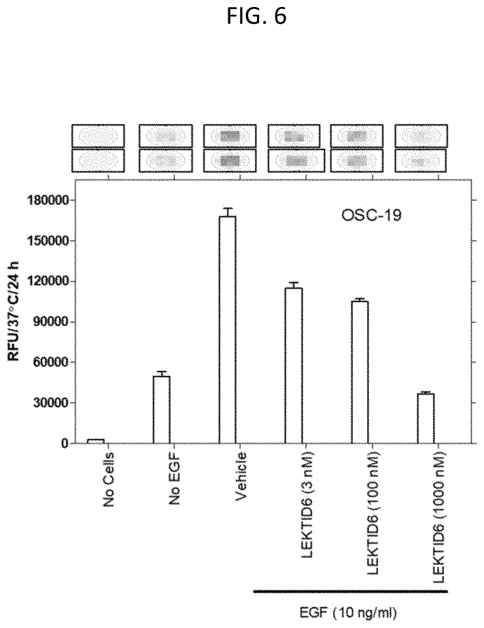

To measure migration alone, parallel wells were set up with control inserts that lacked a Matrigel coating. In this experiment, OSC19 cells were not pretreated with LEKTI D6 before the commencement of invasion. As shown in FIG. 4, OSC19 cells show an increase in invasion in response to EGF as a chemoattractant. OSC19 cells showed a seven or four fold increase in invasion in response to NIH3T3 supernatant plus 10 or 50 ng/ml EGF, respectively, compared to NIH3T3 supernatant without EGF as a chemoattractant. OSC-19 cells show reduced invasion in presence of rLEKTI D6. Further, the effect of exogenous LEKTI D6 on EGF-induced invasion of OSC-19 cells was found to be dose dependent, as shown in FIG. 6.

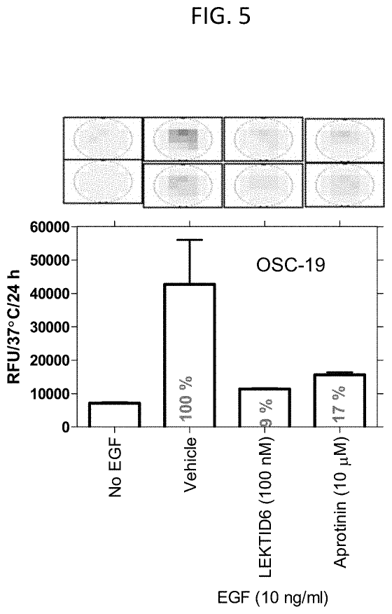

As shown in FIG. 5, OSC19 cells pretreated with LEKTI D6 or Aprotinin, a potent inhibitor of hKLK5, for 24 hours showed 9 or 17% invasion respectively compared to 64% invasion without pretreatment with LEKTI D6.

Literature has shown that full length LEKTI containing 15 domains show no inhibition of invasion of OSC19 cells without pretreatment for at least 6 h with rLEKTI. However, these results show that S. epidermidis (SE) produced LEKTI D6 is more potent than full length LEKTI produced by insect cells. These results were surprising because it would be expected that full length LEKTI, which has a broader array of SERPIN activities, would have been expected to be more potent.

Next, the effect of cell density and EGF on NHEK migration was examined. NHEK cells were grown in the keratinocyte growth medium 2 with supplements, washed with PBS, and suspended in keratinocyte growth medium 2 without supplements are added to the upper chambers (5K, 10K, 20K per well) with or without EGF. The lower chamber contained 500 μl NIH/3T3 supernatant only. After 24 h of incubation the migration of NHEK cells was measured. The results are shown in FIG. 7. In a similar set of experiments, the effect of escalating doses of LEKTID6 on NHEK migration was examined. Briefly, NHEK cells were grown in the keratinocyte growth medium 2 with supplements, washed with PBS, and suspended in keratinocyte growth medium 2 without supplements are added to the upper chambers with or without EGF or with or without LEKTID6. The lower chamber contained 500 μl NIH/3T3 supernatant only. After 24 h of incubation the migration of NHEK cells was measured. The results are shown in FIG. 8.

Cell Proliferation Assay

The ability of cell proliferation can be measured by a CCK-8 assay and plate colony formation assay. The procedure of the CCK8 assay is as follows. The cells in the logarithmic growth phase are seeded at a density of 1×103 per well in 96-well plates, and the proliferation of cells in each treatment group is detected at 0 h, 24 h, 48 h using the CCK8 detection kit. For the plate colony formation assay, the cells in the logarithmic growth phase are inoculated into the 6-well plate at a density of 200 per well. After further culture for 1 week, the cells are stained with crystal violet solution, and the growth of the cells in each treatment group is observed. Cell counting is performed by image J software.

### Example 5. In Vivo Xenograft Model

An orthotopic nude mouse model of HNSCC will be used to examine the effect of LEKTI D6 on tumor growth. OSC-19 cells will be harvested from subconfluent cultures by trypsinization and washed with PBS. An orthotopic nude mouse model of an oral tongue tumor will be established by injecting 1×10⁵OSC-19 cells suspended in 30 mL of serum-free DMEM into the tongues of mice as described previously (Myers et al., an orthotopic nude mouse model of oral tongue squamous cell carcinoma. Clin Cancer Res 2002; 8:293-8). Eight to 10 days after the cells are injected, mice in groups of 6-8 will be assigned to a control group or treatment groups (LEKTI D6). Mice will be examined at least four times a week for tumor size and weight loss. Tumor size will be measured with microcalipers. Mice will be euthanized if they lose 20% or more of their body weight, or if tumors reach a certain pre-determined size.

It is expected that mice injected with LEKTI D6 will show a reduced tumor size compared to control treated mice.

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

Although illustrative embodiments of the present invention have been described herein, it should be understood that the invention is not limited to those described, and that various other changes or modifications may be made by one skilled in the art without departing from the scope or spirit of the invention.

1. A method of treating a subject afflicted with a cancer, comprising administering one or more LEKTI protein domains to the subject in need thereof.

2. The method of claim 1, wherein treating a subject afflicted with cancer comprises:
preventing the recurrence of the cancer in the subject afflicted with the cancer, wherein the cancer is in remission;
preventing the progression of the cancer in the subject afflicted with the cancer; and/or
inhibiting serine protease activity of at least one serine protease in a subject afflicted with cancer.

3.-5. (canceled)

6. The method of claim 1, wherein the one or more LEKTI protein domains are encoded by a nucleic acid.

7. The method of claim 6, wherein the nucleic acid comprises a sequence that is at least 85% identical to SEQ ID NO: 119 or SEQ ID NO: 128, or fragments thereof.

8. The method of claim 6, wherein the nucleic acid is comprised in a vector.

9. The method of claim 8, wherein the vector is a viral expression vector.

10. The method of claim 9, wherein the vector is comprised within a cell.

11. A method of treating a subject afflicted with a cancer, comprising administering a microbe genetically modified to express one or more LEKTI protein domains encoded by one or more SPINK genes to the subject in need thereof.

12. The method of claim 11, wherein treating the subject afflicted with cancer comprises:
preventing the recurrence of the cancer in the subject afflicted with the cancer, wherein the cancer is in remission;
preventing the progression of the cancer in the subject afflicted with the cancer; and/or
inhibiting serine protease activity of at least one serine protease in a subject afflicted with cancer.

13.-15. (canceled)

16. The method of claim 11, wherein the one or more SPINK genes are selected from the group consisting of: SPINK1, SPINK2, SPINK4, SPINK5, SPINK6, SPINK7, SPINK8, SPINK9, SPINK13, and SPINK14.

17. The method of claim 11, wherein the one or more SPINK genes encodes a LEKTI protein, and protein domains thereof, selected from LEKTI, LEKTI-2 and LEKTI-3.

18.-24. (canceled)

25. The method of claim 11, wherein the microbe is adapted to live for a controlled duration on the surface of the mammal's skin to provide a continuous supply of LEKTI protein domains.

26. The method of claim 11, wherein the microbe is genetically modified by transfection/transformation with a recombinant DNA plasmid encoding the LEKTI protein domains.

27. The method of claim 1, wherein:
the LEKTI domains are operably linked to one or more recombinant protein domains that are effective to enhance secretion from the microbe and/or penetration of the mammal's skin;
at least one LEKTI domain is operably linked to a SecA domain; and/or
at least one LEKTI domain is operably linked to an RMR domain.

28. (canceled)

29. (canceled)

30. The method of claim 1, wherein at least one LEKTI domain comprises an amino acid sequence comprising any one of SEQ ID NOs 104-118.

31. The method of claim 11, wherein the microbe is adapted to multiply on the skin of the mammal.

32. The method of claim 1, wherein expression of at least one LEKTI domain is controlled by an operon and the amount of LEKTI provided to the subject's skin is proportional to the availability of an extrinsic factor.

33. (canceled)

34. The method of claim 1, wherein the microbe has been genetically modified by transfection/transformation with a recombinant DNA plasmid encoding the one or more LEKTI protein domains and one or more antibiotic resistance genes.

35. The method of claim 11, wherein the microbe is selected from the group consisting of Acinetobacter spp., Alloiococcus spp., Bifidobacterium spp., Brevibacterium spp., Clostridium spp., Corynebacterium spp., Haemophilus spp., Pseudomonas spp., Propionibacterium spp., Lactococcus spp., Streptococcus spp., Salmonella spp., Staphylococcus spp., Lactobacillus spp., Pediococcus spp., Leuconostoc spp., Moraxella spp., or Oenococcus spp., and mixtures thereof.

36. A recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide.

37. A pharmaceutical composition comprising the recombinant microorganism of claim 36.


SEQUENCE LISTING (128 sequences)

SEQ ID NO: 1-8 - skipped (8 entries)

SEQ ID NO: 9 - protein, 25 aa, Artificial Sequence
  Description of Artificial Sequence: Synthetic peptide
MKKLAFAITAASGAAAVLSHHDAEA

SEQ ID NO: 10 - protein, 30 aa, Artificial Sequence
  Description of Artificial Sequence: Synthetic polypeptide
WLDNRAFSKKFVPVVMATSVALFFLNLAFA

SEQ ID NO: 11 - protein, 29 aa, Artificial Sequence
  Description of Artificial Sequence: Synthetic peptide
MAKKFNYKLPSMVALTLFGTAFTAHQANA

SEQ ID NO: 12 - protein, 27 aa, Artificial Sequence
  Description of Artificial Sequence: Synthetic peptide
MKKRFLSICTMTIAALATTTMVNTSYA

SEQ ID NO: 13 - protein, 30 aa, Artificial Sequence
  Description of Artificial Sequence: Synthetic polypeptide
NLKKQSKLILIFICIFTFFIMIIQSQFLMG

SEQ ID NO: 14 - protein, 26 aa, Artificial Sequence
  Description of Artificial Sequence: Synthetic peptide
MKIFKLTSLTLAALTLAFPFSHVAQA

SEQ ID NO: 15 - protein, 27 aa, Artificial Sequence
  Description of Artificial Sequence: Synthetic peptide
MKKTVIASTLAVSLGIAGYGLSGHEAH

SEQ ID NO: 16 - protein, 27 aa, Artificial Sequence
  Description of Artificial Sequence: Synthetic peptide
MKKNKFLVYLLSTALITPTFATQTAFA

SEQ ID NO: 17 - protein, 35 aa, Artificial Sequence
  Description of Artificial Sequence: Synthetic polypeptide
MKTRQNKYSIRKFSVGASSILIAALLFMGGGSAQA

SEQ ID NO: 18 - protein, 37 aa, Artificial Sequence
  Description of Artificial Sequence: Synthetic polypeptide
MKNNNETRRFSIRKYTVGVVSIITGITIFVSGQHAQA

SEQ ID NO: 19 - protein, 30 aa, Artificial Sequence
  Description of Artificial Sequence: Synthetic polypeptide
MKKKLSYMITIMLAFTLSLALGLFFNSAHA

SEQ ID NO: 20 - protein, 45 aa, Artificial Sequence
  Description of Artificial Sequence: Synthetic polypeptide
MKKRRQGPINKRVDFLSNKVNKYSIRKFTVGTASILVGATLMFGA

SEQ ID NO: 21 - protein, 27 aa, Artificial Sequence
  Description of Artificial Sequence: Synthetic peptide
MKKRFLSICTMTIAALATTTMVNTSYA

SEQ ID NO: 22 - protein, 29 aa, Artificial Sequence
  Description of Artificial Sequence: Synthetic peptide
MAKKFNYKLPSMVALTLFGTAFTAHQANA

SEQ ID NO: 23 - protein, 50 aa, Artificial Sequence
  Description of Artificial Sequence: Synthetic polypeptide
MIKKNNLLTKKKPIANKSNKYAIRKFTVGTASIVIGAALLFGLGHNEAKA

SEQ ID NO: 24 - protein, 30 aa, Artificial Sequence
  Description of Artificial Sequence: Synthetic polypeptide
MKPFKLIFISALMILIMTNATPISHLNAQA

SEQ ID NO: 25 - protein, 35 aa, Artificial Sequence; identical to SEQ ID NO: 17
  Description of Artificial Sequence: Synthetic polypeptide

SEQ ID NO: 26 - protein, 23 aa, Artificial Sequence
  Description of Artificial Sequence: Synthetic peptide
MNKFKFFIVFLILSLVFLQNEYA

SEQ ID NO: 27 - protein, 51 aa, Artificial Sequence
  Description of Artificial Sequence: Synthetic polypeptide
MINKKNNLLTKKKPIANKSNKYAIRKFTVGTASIVIGATLLFGLGHNEAKA

SEQ ID NO: 28 - protein, 26 aa, Artificial Sequence
  Description of Artificial Sequence: Synthetic peptide
MKKIATATIATAGIATFAFAHHDAQA

SEQ ID NO: 29 - protein, 28 aa, Artificial Sequence
  Description of Artificial Sequence: Synthetic peptide
MKNFSKFALTSIAALTVASPLVNTEVDA

SEQ ID NO: 30 - protein, 37 aa, Artificial Sequence; identical to SEQ ID NO: 18
  Description of Artificial Sequence: Synthetic polypeptide

SEQ ID NO: 31 - protein, 19 aa, Artificial Sequence
  Description of Artificial Sequence: Synthetic peptide
MRYLKRITIYISLLILVSG

SEQ ID NO: 32 - protein, 20 aa, Artificial Sequence
  Description of Artificial Sequence: Synthetic peptide
MKLMNKIIVPVTASALLLGA

SEQ ID NO: 33 - protein, 40 aa, Artificial Sequence
  Description of Artificial Sequence: Synthetic polypeptide
MKKIDSWLTKHGLKNRLTLVVIVIFIIFLILLFMFVNLSD

SEQ ID NO: 34 - protein, 19 aa, Artificial Sequence
  Description of Artificial Sequence: Synthetic peptide
MKKKALLPLFLGIMIFLAG

SEQ ID NO: 35 - protein, 28 aa, Artificial Sequence
  Description of Artificial Sequence: Synthetic peptide
MKKTVIASTLAVSLGIAGYGLSGHEAHA

SEQ ID NO: 36 - protein, 19 aa, Artificial Sequence
  Description of Artificial Sequence: Synthetic peptide
MSKFKSLLLLFGTLILLSG

SEQ ID NO: 37 - protein, 27 aa, Artificial Sequence
  Description of Artificial Sequence: Synthetic peptide
MKKTLVASSLAIGLGVVAGNAGHDAHA

SEQ ID NO: 38 - protein, 29 aa, Artificial Sequence
  Description of Artificial Sequence: Synthetic peptide
MAKKFNYKLPSMVALTLFGTAFTAHQANA

SEQ ID NO: 39 - protein, 30 aa, Artificial Sequence; identical to SEQ ID NO: 19
  Description of Artificial Sequence: Synthetic polypeptide

SEQ ID NO: 40 - protein, 18 aa, Artificial Sequence
  Description of Artificial Sequence: Synthetic peptide
MHKRLFITLLGFIILLAG

SEQ ID NO: 41 - protein, 19 aa, Artificial Sequence
  Description of Artificial Sequence: Synthetic peptide
MRYLKRITIYISLLILVSG

SEQ ID NO: 42 - protein, 25 aa, Artificial Sequence
  Description of Artificial Sequence: Synthetic peptide
MQKKYITAIIGTTALSALASTHAQA

SEQ ID NO: 43 - protein, 22 aa, Artificial Sequence
  Description of Artificial Sequence: Synthetic peptide
MKHSSKIIVFVSFLILTIFIGG

SEQ ID NO: 44 - protein, 20 aa, Artificial Sequence
  Description of Artificial Sequence: Synthetic peptide
MKKWQLVGTTVLGASVLLGA

SEQ ID NO: 45 - protein, 52 aa, Artificial Sequence
  Description of Artificial Sequence: Synthetic polypeptide
MGKRRQGPINKKVDFLPNKLNKYSIRKFTVGTASILLGSTLIFGSSSHEAKA

SEQ ID NO: 46 - protein, 26 aa, Artificial Sequence
  Description of Artificial Sequence: Synthetic peptide
MKKIATATIATAGIATFAFAHHDAQA

SEQ ID NO: 47 - protein, 45 aa, Artificial Sequence; identical to SEQ ID NO: 20
  Description of Artificial Sequence: Synthetic polypeptide

SEQ ID NO: 48 - protein, 27 aa, Artificial Sequence
  Description of Artificial Sequence: Synthetic peptide
MKKRFLSICTMTIAALATTTMVNTSYA

SEQ ID NO: 49 - protein, 35 aa, Artificial Sequence; identical to SEQ ID NO: 17
  Description of Artificial Sequence: Synthetic polypeptide

SEQ ID NO: 50 - protein, 28 aa, Artificial Sequence
  Description of Artificial Sequence: Synthetic peptide
MKNFSKFALTSIAALTVASPLVNTEVDA

SEQ ID NO: 51 - protein, 17 aa, Artificial Sequence
  Description of Artificial Sequence: Synthetic peptide
MKKVLASATILSLMLVG

SEQ ID NO: 52 - protein, 22 aa, Artificial Sequence
  Description of Artificial Sequence: Synthetic peptide
MKYYGKCISYISILILTFFIGG

SEQ ID NO: 53 - protein, 22 aa, Artificial Sequence
  Description of Artificial Sequence: Synthetic peptide
MKHSSKIIVFVSFLILTIFIGG

SEQ ID NO: 54 - protein, 30 aa, Artificial Sequence; identical to SEQ ID NO: 24
  Description of Artificial Sequence: Synthetic polypeptide

SEQ ID NO: 55 - protein, 19 aa, Artificial Sequence
  Description of Artificial Sequence: Synthetic peptide
MSKFKSLLLLFGTLILLSG

SEQ ID NO: 56 - protein, 27 aa, Artificial Sequence
  Description of Artificial Sequence: Synthetic peptide
MKKTLVASSLAIGLGVVAGNAGHDAHA

SEQ ID NO: 57 - protein, 19 aa, Artificial Sequence
  Description of Artificial Sequence: Synthetic peptide
MHYLKKVTIYISLLILVSG

SEQ ID NO: 58 - protein, 25 aa, Artificial Sequence
  Description of Artificial Sequence: Synthetic peptide
MQKKYITAIIGTTALSALASTHAQA

SEQ ID NO: 59 - protein, 22 aa, Artificial Sequence
  Description of Artificial Sequence: Synthetic peptide
MKHSKKLLLCISFLLITFFIGG

SEQ ID NO: 60 - protein, 26 aa, Artificial Sequence
  Description of Artificial Sequence: Synthetic peptide
MKKIATATIATAGIATFAFAHHDAQA

SEQ ID NO: 61 - protein, 19 aa, Artificial Sequence
  Description of Artificial Sequence: Synthetic peptide
MRYLKKVTIYISLLILVSG

SEQ ID NO: 62 - protein, 27 aa, Artificial Sequence
  Description of Artificial Sequence: Synthetic peptide
MKKRFLSICTMTIAALATTTMVNTSYA

SEQ ID NO: 63 - protein, 20 aa, Artificial Sequence
  Description of Artificial Sequence: Synthetic peptide
MKKWQLVGTTVLGASVLLGA

SEQ ID NO: 64 - protein, 29 aa, Artificial Sequence
  Description of Artificial Sequence: Synthetic peptide
MAKKFNYKLPSMVALTLFGTAFTAHQANA

SEQ ID NO: 65 - protein, 30 aa, Artificial Sequence; identical to SEQ ID NO: 19
  Description of Artificial Sequence: Synthetic polypeptide

SEQ ID NO: 66 - protein, 18 aa, Artificial Sequence
  Description of Artificial Sequence: Synthetic peptide
MHKRLFITLLGFIILLAG

SEQ ID NO: 67 - protein, 22 aa, Artificial Sequence
  Description of Artificial Sequence: Synthetic peptide
MRYLKKVTIYISLLILTIFIGG

SEQ ID NO: 68 - protein, 17 aa, Artificial Sequence
  Description of Artificial Sequence: Synthetic peptide
MKKVLASATILSLMLVG

SEQ ID NO: 69 - protein, 22 aa, Artificial Sequence
  Description of Artificial Sequence: Synthetic peptide
MKHSKKLLLCISFLLITVFISG

SEQ ID NO: 70 - protein, 22 aa, Artificial Sequence
  Description of Artificial Sequence: Synthetic peptide
MKHSKKLLLCISFLLITFFISG

SEQ ID NO: 71 - protein, 28 aa, Artificial Sequence
  Description of Artificial Sequence: Synthetic peptide
MKKTVIASTLAVSLGIAGYGLSGHEAHA

SEQ ID NO: 72 - protein, 22 aa, Artificial Sequence
  Description of Artificial Sequence: Synthetic peptide
MKHSKKLLLCISFLLITIFISG

SEQ ID NO: 73 - protein, 40 aa, Artificial Sequence; identical to SEQ ID NO: 33
  Description of Artificial Sequence: Synthetic polypeptide

SEQ ID NO: 74 - protein, 19 aa, Artificial Sequence
  Description of Artificial Sequence: Synthetic peptide
MKKKALLPLFLGIMIFLAG

SEQ ID NO: 75 - protein, 20 aa, Artificial Sequence
  Description of Artificial Sequence: Synthetic peptide
MKLMNKIIVPVTASALLLGA

SEQ ID NO: 76 - protein, 35 aa, Artificial Sequence; identical to SEQ ID NO: 17
  Description of Artificial Sequence: Synthetic polypeptide

SEQ ID NO: 77 - protein, 13 aa, Artificial Sequence
  Description of Artificial Sequence: Synthetic peptide
GRKKRRQRRRPPQ

SEQ ID NO: 78 - protein, 27 aa, Artificial Sequence
  Description of Artificial Sequence: Synthetic peptide
GWTLNSAGYLLGKINLKALAALAKKIL

SEQ ID NO: 79 - protein, 18 aa, Artificial Sequence
  Description of Artificial Sequence: Synthetic peptide
KLALKLALKALKAALKLA

SEQ ID NO: 80 - protein, 30 aa, Artificial Sequence
  Description of Artificial Sequence: Synthetic polypeptide
WEAKLAKALAKALAKHLAKALAKALKACEA

SEQ ID NO: 81 - protein, 21 aa, Artificial Sequence
  Description of Artificial Sequence: Synthetic peptide
KETWWETWWTEWSQPKKKRKV

SEQ ID NO: 82 - protein, 9 aa, Artificial Sequence
  Description of Artificial Sequence: Synthetic peptide
RRRRRRRRR

SEQ ID NO: 83 - protein, 24 aa, Artificial Sequence
  Description of Artificial Sequence: Synthetic peptide
LGTYTQDFNKFHTFPQTAIGVGAP

SEQ ID NO: 84 - protein, 15 aa, Artificial Sequence
  Description of Artificial Sequence: Synthetic peptide
RQIKWFQNRRMKWKK

SEQ ID NO: 85 - protein, 11 aa, Artificial Sequence
  Description of Artificial Sequence: Synthetic peptide
YGRKKRRQRRR

SEQ ID NO: 86 - protein, 18 aa, Artificial Sequence
  Description of Artificial Sequence: Synthetic peptide
RGGRLSYSRRRFSTSTGR

SEQ ID NO: 87 - protein, 10 aa, Artificial Sequence
  Description of Artificial Sequence: Synthetic peptide
RRLSYSRRRF

SEQ ID NO: 88 - protein, 12 aa, Artificial Sequence
  Description of Artificial Sequence: Synthetic peptide
PIRRRKKLRRLK

SEQ ID NO: 89 - protein, 12 aa, Artificial Sequence
  Description of Artificial Sequence: Synthetic peptide
RRQRRTSKLMKR

SEQ ID NO: 90 - protein, 15 aa, Artificial Sequence
  Description of Artificial Sequence: Synthetic peptide
RRRRNRTRRNRRRVR

SEQ ID NO: 91 - protein, 19 aa, Artificial Sequence
  Description of Artificial Sequence: Synthetic peptide
KMTRAQRRAAARRNRWTAR

SEQ ID NO: 92 - protein, 13 aa, Artificial Sequence
  Description of Artificial Sequence: Synthetic peptide
TRRQRTRRARRNR

SEQ ID NO: 93 - protein, 13 aa, Artificial Sequence
  Description of Artificial Sequence: Synthetic peptide
GRKKRRQRRRPPQ

SEQ ID NO: 94 - protein, 13 aa, Artificial Sequence
  Description of Artificial Sequence: Synthetic peptide
GRRRRRRRRRPPQ

SEQ ID NO: 95 - protein, 27 aa, Artificial Sequence
  Description of Artificial Sequence: Synthetic peptide
GWTLNSAGYLLGKINLKALAALAKKIL

SEQ ID NO: 96 - protein, 17 aa, Artificial Sequence
  Description of Artificial Sequence: Synthetic peptide
KLALKLALKLALALKLA

SEQ ID NO: 97 - protein, 27 aa, Artificial Sequence
  Description of Artificial Sequence: Synthetic peptide
MGLGLHLLVLAAALQGAWSQPKKKRKV

SEQ ID NO: 98 - protein, 27 aa, Artificial Sequence
  Description of Artificial Sequence: Synthetic peptide
GALFLGWLGAAGSTMGAWSQPKKKRKV

SEQ ID NO: 99 - protein, 27 aa, Artificial Sequence
  Description of Artificial Sequence: Synthetic peptide
GALFLGFLGAAGSTMGAWSQPKKKRKV

SEQ ID NO: 100 - protein, 27 aa, Artificial Sequence
  Description of Artificial Sequence: Synthetic peptide
GALFLGFLGAAGSTMGAWSQPKSKRKV

SEQ ID NO: 101 - protein, 21 aa, Artificial Sequence
  Description of Artificial Sequence: Synthetic peptide
KETWWETWWTEWSQPKKKRKV

SEQ ID NO: 102 - protein, 21 aa, Artificial Sequence
  Description of Artificial Sequence: Synthetic peptide
KETWFETWFTEWSQPKKKRKV

SEQ ID NO: 103 - protein, 1064 aa, Homo sapiens
MKIATVSVLLPLALCLIQDAASKNEDQEMCHEFQAFMKNGKLFCPQDKKFFQSLDGIMFINKCATCKMILEKEAKSQKRARHLARAPKATAPTELNCDDFKKGERDGDFICPDYYEAVCGTDGKTYDNRCALCAENAKTGSQIGVKSEGECKSSNPEQDVCSAFRPFVRDGRLGCTRENDPVLGPDGKTHGNKCAMCAELFLKEAENAKREGETRIRRNAEKDFCKEYEKQVRNGRLFCTRESDPVRGPDGRMHGNKCALCAEIFKQRFSEENSKTDQNLGKAEEKTKVKREIVKLCSQYQNQAKNGILFCTRENDPIRGPDGKMHGNLCSMCQAYFQAENEEKKKAEARARNKRESGKATSYAELCSEYRKLVRNGKLACTRENDPIQGPDGKVHGNTCSMCEVFFQAEEEEKKKKEGKSRNKRQSKSTASFEELCSEYRKSRKNGRLFCTRENDPIQGPDGKMHGNTCSMCEAFFQQEERARAKAKREAAKEICSEFRDQVRNGTLICTREHNPVRGPDGKMHGNKCAMCASVFKLEEEEKKNDKEEKGKVEAEKVKREAVQELCSEYRHYVRNGRLPCTRENDPIEGLDGKIHGNTCSMCEAFFQQEAKEKERAEPRAKVKREAEKETCDEFRRLLQNGKLFCTRENDPVRGPDGKTHGNKCAMCKAVFQKENEERKRKEEEDQRNAAGHGSSGGGGGNTQDECAEYREQMKNGRLSCTRESDPVRDADGKSYNNQCTMCKAKLEREAERKNEYSRSRSNGTGSESGKDTCDEFRSQMKNGKLICTRESDPVRGPDGKTHGNKCTMCKEKLEREAAEKKKKEDEDRSNTGERSNTGERSNDKEDLCREFRSMQRNGKLICTRENNPVRGPYGKMHINKCAMCQSIFDREANERKKKDEEKSSSKPSNNAKDECSEFRNYIRNNELICPRENDPVHGADGKFYTNKCYMCRAVFLTEALERAKLQEKPSHVRASQEEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 104 - protein, 55 aa, Homo sapiens
KNEDQEMCHEFQAFMKNGKLFCPQDKKFFQSLDGIMFINKCATCKMILEKEAKSQ

SEQ ID NO: 105 - protein, 63 aa, Homo sapiens
APTELNCDDFKKGERDGDFICPDYYEAVCGTDGKTYDNRCALCAENAKTGSQIGVKSEGECKS

SEQ ID NO: 106 - protein, 62 aa, Homo sapiens
NPEQDVCSAFRPFVRDGRLGCTRENDPVLGPDGKTHGNKCAMCAELFLKEAENAKREGETRI

SEQ ID NO: 107 - protein, 67 aa, Homo sapiens
NAEKDFCKEYEKQVRNGRLFCTRESDPVRGPDGRMHGNKCALCAEIFKQRFSEENSKTDQNLGKAEE

SEQ ID NO: 108 - protein, 62 aa, Homo sapiens
REIVKLCSQYQNQAKNGILFCTRENDPIRGPDGKMHGNLCSMCQAYFQAENEEKKKAEARAR

SEQ ID NO: 109 - protein, 68 aa, Homo sapiens
ESGKATSYAELCSEYRKLVRNGKLACTRENDPIQGPDGKVHGNTCSMCEVFFQAEEEEKKKKEGKSRN

SEQ ID NO: 110 - protein, 59 aa, Homo sapiens
ASFEELCSEYRKSRKNGRLFCTRENDPIQGPDGKMHGNTCSMCEAFFQQEERARAKAKR

SEQ ID NO: 111 - protein, 62 aa, Homo sapiens
EAAKEICSEFRDQVRNGTLICTREHNPVRGPDGKMHGNKCAMCASVFKLEEEEKKNDKEEKG

SEQ ID NO: 112 - protein, 62 aa, Homo sapiens
EAVQELCSEYRHYVRNGRLPCTRENDPIEGLDGKIHGNTCSMCEAFFQQEAKEKERAEPRAK

SEQ ID NO: 113 - protein, 63 aa, Homo sapiens
EAEKETCDEFRRLLQNGKLFCTRENDPVRGPDGKTHGNKCAMCKAVFQKENEERKRKEEEDQR

SEQ ID NO: 114 - protein, 57 aa, Homo sapiens
GNTQDECAEYREQMKNGRLSCTRESDPVRDADGKSYNNQCTMCKAKLEREAERKNEY

SEQ ID NO: 115 - protein, 63 aa, Homo sapiens
ESGKDTCDEFRSQMKNGKLICTRESDPVRGPDGKTHGNKCTMCKEKLEREAAEKKKKEDEDRS

SEQ ID NO: 116 - protein, 63 aa, Homo sapiens
NDKEDLCREFRSMQRNGKLICTRENNPVRGPYGKMHINKCAMCQSIFDREANERKKKDEEKSS

SEQ ID NO: 117 - protein, 62 aa, Homo sapiens
NNAKDECSEFRNYIRNNELICPRENDPVHGADGKFYTNKCYMCRAVFLTEALERAKLQEKPS

SEQ ID NO: 118 - protein, 62 aa, Homo sapiens
SLDSEMCKDYRVLPRIGYLCPKDLKPVCGDDGQTYNNPCMLCHENLIRQTNTHIRSTGKCEE

SEQ ID NO: 119 - DNA, 3192 bp, Homo sapiens
atgaagatagccacagtgtcagtgcttctgcccttggctctttgcctcatacaagatgctgccagtaagaatgaagatcaggaaatgtgccatgaatttcaggcatttatgaaaaatggaaaactgttctgtccccaggataagaaattttttcaaagtcttgatggaataatgttcatcaataaatgtgccacgtgcaaaatgatactggaaaaagaagcaaaatcacagaagagggccaggcatttagcaagagctcccaaggctactgccccaacagagctgaattgtgatgattttaaaaaaggagaaagagatggggattttatctgtcctgattattatgaagctgtttgtggcacagatgggaaaacatatgacaacagatgtgcactgtgtgctgagaatgcgaaaaccgggtcccaaattggtgtaaaaagtgaaggggaatgtaagagcagtaatccagagcaggatgtatgcagtgcttttcggccctttgttagagatggaagacttggatgcacaagggaaaatgatcctgttcttggtcctgatgggaagacgcatggcaataagtgtgcaatgtgtgctgagctgtttttaaaagaagctgaaaatgccaagcgagagggtgaaactagaattcgacgaaatgctgaaaaggatttttgcaaggaatatgaaaaacaagtgagaaatggaaggcttttttgtacacgggagagtgatccagtccgtggccctgacggcaggatgcatggcaacaaatgtgccctgtgtgctgaaattttcaagcagcgtttttcagaggaaaacagtaaaacagatcaaaatttgggaaaagctgaagaaaaaactaaagttaaaagagaaattgtgaaactctgcagtcaatatcaaaatcaggcaaagaatggaatacttttctgtaccagagaaaatgaccctattcgtggtccagatgggaaaatgcatggcaacttgtgttccatgtgtcaagcctacttccaagcagaaaatgaagaaaagaaaaaggctgaagcacgagctagaaacaaaagagaatctggaaaagcaacctcatatgcagagctttgcagtgaatatcgaaagcttgtgaggaacggaaaacttgcttgcaccagagagaacgatcctatccagggcccagatgggaaagtgcatggcaacacctgctccatgtgtgaggtcttcttccaagcagaagaagaagaaaagaaaaagaaggaaggtaaatcaagaaacaaaagacaatctaagagtacagcttcctttgaggagttgtgtagtgaataccgcaaatccaggaaaaacggacggcttttttgcaccagagagaatgaccccatccagggcccagatggaaaaatgcatggcaacacctgctccatgtgtgaggccttctttcaacaagaagaaagagcaagagcaaaggctaaaagagaagctgcaaaggaaatctgcagtgaatttcgggaccaagtgaggaatggaacacttatatgcaccagggagcataatcctgtccgtggcccagatggcaaaatgcatggaaacaagtgtgccatgtgtgccagtgtgttcaaacttgaagaagaagagaagaaaaatgataaagaagaaaaagggaaagtcgaggctgaaaaagttaagagagaagcagttcaggagctgtgcagtgaatatcgtcattatgtgaggaatggacgactcccctgtaccagagagaatgatcctattgagggtctagatgggaaaatccacggcaacacctgctccatgtgtgaagccttcttccagcaagaagcaaaagaaaaagaaagagctgaacccagagcaaaagtcaaaagagaagctgaaaaggagacatgcgatgaatttcggagacttttgcaaaatggaaaacttttctgcacaagagaaaatgatcctgtgcgtggcccagatggcaagacccatggcaacaagtgtgccatgtgtaaggcagtcttccagaaagaaaatgaggaaagaaagaggaaagaagaggaagatcagagaaatgctgcaggacatggttccagtggtggtggaggaggaaacactcaggacgaatgtgctgagtatcgggaacaaatgaaaaatggaagactcagctgtactcgggagagtgatcctgtacgtgatgctgatggcaaatcgtacaacaatcagtgtaccatgtgtaaagcaaaattggaaagagaagcagagagaaaaaatgagtattctcgctccagatcaaatgggactggatcagaatcagggaaggatacatgtgatgagtttagaagccaaatgaaaaatggaaaactcatctgcactcgagaaagtgaccctgtccggggtccagatggcaagacacatggcaataagtgtactatgtgtaaggaaaaactggaaagggaagcagctgaaaaaaaaaagaaagaggatgaagacaggagcaatacaggagaaaggagcaatacaggagaaaggagcaatgacaaagaggatctgtgtcgtgaatttcgaagcatgcagagaaatggaaagcttatctgcaccagagaaaataaccctgttcgaggcccatatggcaagatgcacatcaataaatgtgctatgtgtcagagcatctttgatcgagaagctaatgaaagaaaaaagaaagatgaagagaaatcaagtagcaagccctcaaataatgcaaaggatgagtgcagtgaatttcgaaactatataaggaacaatgaactcatctgccctagagagaatgacccagtgcacggtgctgatggaaagttctatacaaacaagtgctacatgtgcagagctgtctttctaacagaagctttggaaagggcaaagcttcaagaaaagccatcccatgttagagcttctcaagaggaagacagcccagactctttcagttctctggattctgagatgtgcaaagactaccgagtattgcccaggataggttatctttgtccaaaggatttaaagcctgtctgtggtgacgatggccaaacctacaacaatccttgcatgctctgtcatgaaaacctgatacgccaaacaaatacacacatccgcagtacagggaagtgtgaggagagcagcaccccaggaaccaccgcagccagcatgcccccgtctgacgaa

SEQ ID NO: 120 - protein, 6 aa, Artificial Sequence
  Description of Artificial Sequence: Synthetic 6xHis tag
HHHHHH

SEQ ID NO: 121 - protein, 4 aa, Artificial Sequence
  Description of Artificial Sequence: Synthetic peptide
AAPF

SEQ ID NO: 122 - DNA, 21 bp, Artificial Sequence
  Description of Artificial Sequence: Synthetic oligonucleotide
atgcgtcgtatgcgtcgtatg

SEQ ID NO: 123 - DNA, 18 bp, Artificial Sequence
  Description of Artificial Sequence: Synthetic oligonucleotide
gtggtggtggtggtggtg

SEQ ID NO: 124 - DNA, 22 bp, Artificial Sequence
  Description of Artificial Sequence: Synthetic oligonucleotide
ttgatatgcctcctaaattttt

SEQ ID NO: 125 - DNA, 21 bp, Artificial Sequence
  Description of Artificial Sequence: Synthetic oligonucleotide
taggcgcgcctattctaatgc

SEQ ID NO: 126 - protein, 17 aa, Artificial Sequence
  Description of Artificial Sequence: Synthetic peptide
  MISC_FEATURE (1)..(17): This sequence may encompass 4-17 residues
RRRRRRRRRRRRRRRRR

SEQ ID NO: 127 - protein, 17 aa, Artificial Sequence
  Description of Artificial Sequence: Synthetic peptide
  MISC_FEATURE (1)..(17): This sequence may encompass 4-17 residues
KKKKKKKKKKKKKKKKK

SEQ ID NO: 128 - DNA, 207 bp, Homo sapiens
gaatctggtaaagcaacgagttacgctgaattgtgctcagaataccgtaagttagtcagaaacggaaagttggcgtgtactcgtgagaacgaccctatccagggaccggacggcaaggtacatggaaatacttgcagtatgtgcgaggtgtttttccaggcggaggaagaagaaaagaagaaaaaggagggcaaaagtcgaaattaa